(19) United States
(12) Patent Application Publication
(10) Pub. No.: US 2020/0353062 A1
(43) Pub. Date: Nov. 12, 2020

(54) PHARMACEUTICAL COMPOSITIONS, METHODS FOR PREPARATION COMPRISING SIZING OF LIPID VESICLE PARTICLES, AND USES THEREOF

(71) Applicant: IMMUNOVACCINE TECHNOLOGIES INC., Dartmouth (CA)

(72) Inventors: Leeladhar SAMMATUR, Halifax (CA); Rajkannan RAJAGOPALAN, Dartmouth (CA); Arthvan SHARMA, Halifax (CA); Valarmathy KALIAPERUMAL, Halifax (CA); Genevieve WEIR, Lawrencetown (CA); Marianne STANFORD, Upper Tantallon (CA); Andrea PENWELL, Fall River (CA)

(73) Assignee: IMMUNOVACCINE TECHNOLOGIES INC., Dartmouth, NS (CA)

(21) Appl. No.: 16/762,286

(22) PCT Filed: Nov. 9, 2017
(86) PCT No.: PCT/CA2017/051335

§ 371 Date: May 7, 2020

(51) Int. Cl. A61K 39/00; A61K 47/26; A61K 9/107; A61K 9/127; A61K 9/19; A61K 9/51; A61K 47/54

(52) U.S. Cl. CPC A61K 39/00115; A61K 47/26; A61K 9/107; A61K 9/0019; A61K 9/19; A61K 9/5123; A61K 47/543; A61K 9/127

(57) ABSTRACT

The present disclosure relates to methods for preparing a dried preparation comprising lipids and therapeutic agents whereby the therapeutic agents are incorporated both before and after sizing of lipid vesicle particles to a mean particle size of ≤120 nm and a polydispersity index (PDI) of ≤0.1. The present application also provides stable, water-free pharmaceutical compositions comprising one or more lipid-based structures having a single layer lipid assembly, at least two therapeutic agents, and a hydrophobic carrier, as well as methods of treatment, uses and kits relating thereto, such as for example for inducing an antibody and/or CTL immune response.

### FIELD

The present application relates to methods for preparing a dried preparation comprising lipids and therapeutic agents, methods for preparing pharmaceutical compositions, and stable, water-free pharmaceutical compositions comprising one or more lipid-based structures having a single layer lipid assembly, at least two therapeutic agents, and a hydrophobic carrier.

### BACKGROUND

In the pharmaceutical field, the effective delivery of therapeutic agents often poses difficulties and challenges, particularly in respect of the complexities of emerging delivery platforms designed to enhance the efficacy of therapeutic agents. For these specialized delivery platforms that employ unique components, new hurdles arise that do not exist for conventional pharmaceutical compositions. This is certainly the case for delivery platforms using water-free, hydrophobic carriers.

Various characteristics of therapeutic agents make their incorporation into such delivery platforms a challenging task. For instance, because of the high degree of hydrophilicity or hydrophobicity of many therapeutic agents, manufacturing processes involving sequential stages of preparation in both aqueous and hydrophobic solutions create unique obstacles for preparing pharmaceutical grade formulations. Moreover, encapsulation of therapeutic agents in liposomal delivery vehicles means size extrusion steps are often required in order to effectively perform sterile filtration procedures to obtain pharmaceutical grade compositions. However, the sensitivity of some therapeutic agents to these size extrusion steps can result in a lack of reproducibility and/or an unacceptable composition for pharmaceutical purposes.

As such, there remains a need for suitable manufacturing methods involving size extrusion protocols which reproducibly formulate therapeutic agents in stable and immunologically effective pharmaceutical compositions. There also remains a need for stable and effective water-free pharmaceutical compositions comprising multiple therapeutic agents.

### SUMMARY

In an embodiment, the present disclosure relates to a method for preparing a dried preparation comprising lipids and therapeutic agents, said method comprising the steps of: (a) providing a lipid vesicle particle preparation comprising lipid vesicle particles and at least one solubilized first therapeutic agent; (b) sizing the lipid vesicle particle preparation to form a sized lipid vesicle particle preparation comprising sized lipid vesicle particles and said at least one solubilized first therapeutic agent, said sized lipid vesicle particles having a mean particle size of ≤120 nm and a polydispersity index (PDI) of ≤0.1; (c) mixing the sized lipid vesicle particle preparation with at least one second therapeutic agent to form a mixture, wherein said at least one second therapeutic agent is solubilized in the mixture and is different from said at least one solubilized first therapeutic agent; and (d) drying the mixture formed in step (c) to form a dried preparation comprising lipids and therapeutic agents.

In an embodiment, the present disclosure relates to a method for preparing a pharmaceutical composition comprising solubilizing the dried preparation obtained by the method as described herein in a hydrophobic carrier.

In an embodiment, the present disclosure relates to a pharmaceutical composition prepared by the method as disclosed herein.

In an embodiment, the present disclosure relates to a stable, water-free pharmaceutical composition comprising one or more lipid-based structures having a single layer lipid assembly, at least two different therapeutic agents, and a hydrophobic carrier.

In an embodiment, the present disclosure relates to a method of inducing an antibody and/or CTL immune response in a subject comprising administering to the subject the pharmaceutical composition as described herein.

In an embodiment, the present disclosure relates to the use of the pharmaceutical composition as described herein for inducing an antibody and/or CTL immune response in a subject.

In an embodiment, the present disclosure relates to a kit for preparing a pharmaceutical composition for inducing an antibody and/or CTL immune response, the kit comprising: a container comprising a dried preparation prepared by the method as described herein; and a container comprising a hydrophobic carrier.

Other aspects and features of the present invention will become apparent to those of ordinary skill in the art upon review of the following description in conjunction with the accompanying figures.

### BRIEF DESCRIPTION OF THE FIGURES

The accompanying figures, which constitute a part of this specification, illustrate embodiments of the invention by way of example only:

FIG. 1 depicts photographs of pharmaceutical compositions obtained by methods involving: (A) sized lipid vesicle particles (clear), (B) no lipids (turbid), and (C) non-sized lipid vesicle particles (turbid).

FIG. 2 depicts the small angle x-ray scattering (SAXS) pattern for a sample of Montanide ISA 51 VG.

FIG. 3 depicts the SAXS pattern for a sample of Batch #1.

FIG. 4 depicts the pair distance distribution function for the Batch #1 sample at 15.7 cm detector distance.

### DETAILED DESCRIPTION

The present invention relates to advantageous methods for preparing a dried preparation comprising lipids and therapeutic agents, as well as pharmaceutical compositions prepared therefrom. The disclosed methods allow different therapeutic agents to be incorporated into the formulation process at different stages and are capable of providing stable, water-free pharmaceutical compositions.

Method for Preparing a Dried Antigen Preparation

In an embodiment, the present invention relates to a method for preparing a dried preparation comprising lipids and therapeutic agents, said method comprising the steps of: (a) providing a lipid vesicle particle preparation comprising lipid vesicle particles and at least one solubilized first therapeutic agent; (b) sizing the lipid vesicle particle preparation to form a sized lipid vesicle particle preparation comprising sized lipid vesicle particles and said at least one solubilized first therapeutic agent, said sized lipid vesicle particles having a mean particle size of ≤120 nm and a polydispersity index (PDI) of ≤0.1; (c) mixing the sized lipid vesicle particle preparation with at least one second therapeutic agent to form a mixture, wherein said at least one second therapeutic agent is solubilized in the mixture and is different from said at least one solubilized first therapeutic agent; and (d) drying the mixture formed in step (c) to form a dried preparation comprising lipids and therapeutic agents.

As used herein, the term “lipid vesicle particle” may be used interchangeably with “lipid vesicle”. A lipid vesicle particle refers to a complex or structure having an internal environment separated from the external environment by a continuous layer of enveloping lipids. In the context of the present disclosure, the expression “layer of enveloping lipids” can mean a single layer lipid membrane (e.g. as found on a micelle or reverse micelle), a bilayer lipid membrane (e.g. as found on a liposome) or any multilayer membrane formed from single and/or bilayer lipid membranes. The layer of enveloping lipids is typically a single layer, bilayer or multilayer throughout its circumference, but it is contemplated that other conformations may be possible such that the layer has different configurations over its circumference. The lipid vesicle particle may contain, within its internal environment, other vesicle structures (i.e. it may be multivesicular).

The term “lipid vesicle particle” encompasses many different types of structures, including without limitation micelles, reverse micelles, unilamellar liposomes, multilamellar liposomes and multivesicular liposomes.

The lipid vesicle particles may take on various different shapes, and the shape may change at any given time (e.g. upon sizing, mixing with the second therapeutic agent, and/or drying). Typically, lipid vesicle particles are spherical or substantially spherical structures. By “substantially spherical” it is meant that the lipid vesicles are close to spherical, but may not be a perfect sphere. Other shapes of the lipid vesicle particles include, without limitation, oval, oblong, square, rectangular, triangular, cuboid, crescent, diamond, cylinder or hemisphere shapes. Any regular or irregular shape may be formed. Further, a single lipid vesicle particle may comprise different shapes if it is multivesicular. For example, the outer vesicle shape may be oblong or rectangular while an inner vesicle may be spherical.

The lipid vesicle particles are formed from single layer lipid membranes, bilayer lipid membranes and/or multilayer lipid membranes. The lipid membranes are predominantly comprised of and formed by lipids, but may also comprise additional components. For example, and without limitation, the lipid membrane may include stabilizing molecules to aid in maintaining the size and/or shape of the lipid vesicle particle. Any stabilizing molecule known in the art may be used so long as it does not negatively affect the ability of the lipid vesicle particles to be used in the disclosed methods.

The term “lipid” has its common meaning in the art in that it is any organic substance or compound that is soluble in nonpolar solvents, but generally insoluble in polar solvents (e.g. water). Lipids are a diverse group of compounds including, without limitation, fats, waxes, sterols, fat-soluble vitamins, monoglycerides, diglycerides, triglycerides and phospholipids. For the lipid vesicle particles herein, any lipid may be used so long as it is a membrane-forming lipid. By “membrane-forming lipid” it is meant that the lipid, alone or together with other lipids and/or stabilizing molecules, is capable of forming the lipid membrane of the lipid vesicle particle. The lipid vesicle particles may comprise a single type of lipid or two or more different types of lipids.

In an embodiment, the lipid or lipids of the lipid vesicle particle are amphiphilic lipids, meaning that they possess both hydrophilic and hydrophobic (lipophilic) properties.

Although any lipid as defined above may be used, particularly suitable lipids may include those with at least one fatty acid chain containing at least 4 carbons, and typically about 4 to 28 carbons. The fatty acid chain may contain any number of saturated and/or unsaturated bonds. The lipid may be a natural lipid or a synthetic lipid. Non-limiting examples of lipids may include phospholipids, sphingolipids, sphingomyelin, cerobrocides, gangliosides, ether lipids, sterols, cardiolipin, cationic lipids and lipids modified with poly (ethylene glycol) and other polymers. Synthetic lipids may include, without limitation, the following fatty acid constituents: lauroyl, myristoyl, palmitoyl, stearoyl, arachidoyl, oleoyl, linoleoyl, erucoyl, or combinations of these fatty acids.

In an embodiment, the lipid is a phospholipid or a mixture of phospholipids. Broadly defined, a “phospholipid” is a member of a group of lipid compounds that yield on hydrolysis phosphoric acid, an alcohol, fatty acid, and nitrogenous base.

Phospholipids that may be used include for example, and without limitation, those with at least one head group selected from the group consisting of phosphoglycerol, phosphoethanolamine, phosphoserine, phosphocholine (e.g. DOPC; 1,2-Dioleoyl-sn-glycero-3-phosphocholine) and phosphoinositol. In an embodiment, the phospholipid may be phosphatidylcholine or a mixture of lipids comprising phosphatidylcholine. In an embodiment, the lipid may be DOPC (Lipoid GmbH, Germany) or Lipoid S100 lecithin. In some embodiments, a mixture of DOPC and unesterified cholesterol may be used. In other embodiments, a mixture of Lipoid S100 lecithin and unesterified cholesterol may be used.

In an embodiment, the lipid vesicle particles comprise a synthetic lipid. In an embodiment, the lipid vesicle particles comprise synthetic DOPC. In another embodiment, the lipid vesicle particles comprise synthetic DOPC and cholesterol.

When cholesterol is used, the cholesterol may be used in any amount sufficient to stabilize the lipids in the lipid membrane. In an embodiment, the cholesterol may be used in an amount equivalent to about 10% of the weight of phospholipid (e.g. in a DOPC:cholesterol ratio of 10:1 w/w). The cholesterol may stabilize the formation of phospholipid vesicle particles. If a compound other than cholesterol is used, one skilled in the art can readily determine the amount needed.

In an embodiment, the compositions disclosed herein comprise about 120 mg/mL of DOPC and about 12 mg/mL of cholesterol.

Another common phospholipid is sphingomyelin. Sphingomyelin contains sphingosine, an amino alcohol with a long unsaturated hydrocarbon chain. A fatty acyl side chain is linked to the amino group of sphingosine by an amide bond, to form ceramide. The hydroxyl group of sphingosine is esterified to phosphocholine. Like phosphoglycerides, sphingomyelin is amphipathic.

Lecithin, which also may be used, is a natural mixture of phospholipids typically derived from chicken eggs, sheep's wool, soybean and other vegetable sources.

All of these and other phospholipids may be used in the practice of the invention. Phospholipids can be purchased, for example, from Avanti lipids (Alabastar, Ala., USA), Lipoid LLC (Newark, N.J., USA) and Lipoid GmbH (Germany), among various other suppliers.

The lipid vesicle particles are closed vesicular structures. They are typically spherical in shape, but other shapes and conformations may be formed and are not excluded. Exemplary embodiments of lipid vesicle particles include, without limitation, single layer vesicular structures (e.g. micelles) and bilayer vesicular structures (e.g. unilamellar or multilamellar vesicles), or various combinations thereof.

By “single layer” it is meant that the lipids do not form a bilayer, but rather remain in a layer with the hydrophobic part oriented on one side and the hydrophilic part oriented on the opposite side. By “bilayer” it is meant that the lipids form a two-layered sheet, typically with the hydrophobic part of each layer internally oriented toward the center of the bilayer with the hydrophilic part externally oriented. However, the opposite configuration is also possible. The term “multilayer” is meant to encompass any combination of single and bilayer structures. The form adopted may depend upon the specific lipid that is used. Also, in respect of the sized lipid vesicle particles herein, the forms may depend on the size constraints of the disclosed methods, i.e. a mean particle size of ≤120 nm and a PDI of ≤0.1.

In an embodiment, the lipid vesicle particle is a bilayer vesicular structure, such as for example, a liposome. Liposomes are completely closed lipid bilayer membranes. Liposomes may be unilamellar vesicles (possessing a single bilayer membrane), multilamellar vesicles (characterized by multimembrane bilayers whereby each bilayer may or may not be separated from the next by an aqueous layer) or multivesicular vesicles (possessing one or more vesicles within a vesicle). A general discussion of liposomes can be found in Gregoriadis 1990; and Frezard 1999.

Thus, in an embodiment, the lipid vesicle particles are liposomes. In an embodiment, the liposomes are unilamellar, multilamellar, multivesicular or a mixture thereof.

As used herein, the term “therapeutic agent” is any molecule, substance or compound that is capable of providing a therapeutic activity, response or effect in the treatment or prevention of a disease, disorder or condition, including diagnostic and prophylactic agents. As described elsewhere herein, the term “therapeutic agent” does not include or encompass a T-helper epitope or an adjuvant, which are separately described in the present specification and are different components that may or may not be included in the methods, dried preparations, compositions, uses and kits disclosed herein.

In relation to the methods disclosed herein, a “first therapeutic agent” is any one or more therapeutic agents which are used in the preparation of the non-sized lipid vesicle particle preparation (i.e. incorporated in the methods before the step of sizing the non-sized lipid vesicle preparation). In contrast, a “second therapeutic agent” is any one or more therapeutic agents which are used in the methods herein after preparation of the sized lipid vesicle particle preparation (i.e. incorporated in the methods after the step of sizing the non-sized lipid vesicle preparation).

In the practice of the methods disclosed herein, the “first therapeutic agent” and the “second therapeutic agent” are different therapeutic agents, meaning that if a certain therapeutic agent is used as a first therapeutic agent, it is not used again as a second therapeutic agent in the preparation of the same composition. In an embodiment, the second therapeutic agents are of a different type than the first therapeutic agents (e.g. one or more peptide antigens as first therapeutic agents in combination with one or more small molecule drugs as second therapeutic agents, etc.). In another embodiment, the first and second therapeutic agents are all of the same type (e.g. all peptide antigens, all small molecule drugs, all polynucleotides encoding polypeptides, etc.). In yet another embodiment, the first and second therapeutics agents may include some therapeutic agents of the same type and some therapeutic agents of different types, so long as none of the second therapeutic agents are identical to a first therapeutic agent.

In an embodiment, the methods disclosed herein are for formulating multiple different therapeutic agents in a single composition. In an embodiment, the methods disclosed herein are for formulating 2, 3, 4, 5, 6, 7, 8, 9, 10 or more different therapeutic agents in a single composition. In an embodiment, the methods disclosed herein are for formulating 2 to 10 different therapeutic agents in a single composition. In an embodiment, the methods disclosed herein are for formulating 2, 3, 4, or 5 different therapeutic agents in a single composition. In a particular embodiment, the methods disclosed herein are for formulating five different therapeutic agents in a single composition.

In an embodiment, each of the first and second therapeutic agents is independently selected from a peptide antigen, a DNA or RNA polynucleotide that encodes a polypeptide (e.g. mRNA), a hormone, a cytokine, an allergen, a catalytic DNA (deoxyribozyme), a catalytic RNA (ribozyme), an antisense RNA, an interfering RNA (e.g. siRNA or miRNA), an antagomir, a small molecule drug, a biologic drug, an antibody, or a fragment or derivative of any one thereof; or a mixture thereof.

In a particular embodiment, each of the first and second therapeutic agents is a peptide antigen.

The peptide antigen may be a polypeptide of any length. In an embodiment, the peptide antigen may be 5 to 120 amino acids in length, 5 to 100 amino acids in length, 5 to 75 amino acids in length, 5 to 50 amino acids in length, 5 to 40 amino acids in length, 5 to 30 amino acids in length, 5 to 20 amino acids in length or 5 to 10 amino acids in length. In an embodiment, the peptide antigen may be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids in length. In an embodiment, the peptide antigen is 8 to 40 amino acids in length. In an embodiment, the peptide antigen is 9 or 10 amino acids in length.

In a particular embodiment, the first and/or second therapeutic agents are any one or more peptide antigens as described herein.

Further exemplary embodiments of therapeutic agents that may be used in the practice of the methods disclosed herein are described below, without limitation.

Step (a) of the disclosed methods is directed to providing a lipid vesicle particle preparation comprising lipid vesicle particles and at least one first therapeutic agent.

In the practice of the methods disclosed herein, the lipid vesicle particles of the lipid vesicle particle preparation of step (a) may be any of the lipid vesicle particles as described herein. In an embodiment, it is contemplated that prior to step (b) of the methods herein, the lipid vesicle particles may have undergone, or have been subjected to, processing steps that impart some level or degree of sizing, such as for example to provide a mean particle size and/or PDI outside the defined criteria of step (b), i.e. a mean particle size of a certain value>120 nm and/or a PDI of a certain value>0.1. In an embodiment, lipid vesicle particle preparations containing such lipid vesicle particles are encompassed by step (a) of the methods disclosed herein.

In an embodiment, the lipid vesicle particles of the lipid vesicle particle preparation of step (a) are not sized. By this, it is meant that prior to step (b) of the methods herein, the lipid vesicle particles have not undergone, nor have they been subjected to, any processing steps to size the lipid vesicle particles. Thus, in an embodiment, the lipid vesicle particles of the lipid vesicle particle preparation of step (a) are of any size and of any distribution of size. In an embodiment, the lipid vesicle particles of the lipid vesicle particle preparation of step (a) are of a size and size distribution as would naturally result by preparing the lipid vesicle particles as described herein.

For ease of reference in distinguishing the “sized lipid vesicle particles” attained by sizing step (b) in the disclosed methods (i.e. lipid vesicle particles having a mean particle size of ≤120 nm and a PDI of ≤0.1), as used herein the expression “non-sized lipid vesicle particles” refers to any embodiment of the lipid vesicle particles prior to sizing step (b). It should be understood that the expression “non-sized lipid vesicle particles” encompasses both of the embodiments described above whereby the lipid vesicle particles are not sized or the lipid vesicle particles have been subjected to processing steps that impart some level or degree of sizing. The non-sized lipid vesicle particles may be of any size and of any distribution of size.

Likewise, for ease of reference in distinguishing the “sized lipid vesicle particle preparation” of step (b) from the “lipid vesicle particle preparation” of step (a), the preparation of step (a) will be referred to herein as a “non-sized lipid vesicle particle preparation”. However, it should be understood that the expression “non-sized lipid vesicle particle preparation” encompasses both embodiments whereby the lipid vesicle particles contained therein are not sized or the lipid vesicle particles contained therein have been subjected to processing steps that impart some level or degree of sizing. The lipid vesicle particles of the “non-sized lipid vesicle preparation” may be of any size and of any distribution of size.

By “distribution of size” it is meant to refer to polydispersity index (PDI). In respect of the present disclosure, PDI is a measure of the size distribution of the lipid vesicle particles in a mixture. The PDI can be calculated by determining the mean particle size of the lipid vesicle particles and the standard deviation from that size. There are techniques and instruments available for measuring the PDI of lipid vesicle particles. For example, dynamic light scattering (DLS) is a well-established technique for measuring the particle size and size distribution of particles, with available technology to measure particle sizes of less than 1 nm and up to greater than 10 μm (LS Instruments, CH; Malvern Instruments, UK).

For a perfectly uniform sample, the PDI would be 0.0. For a “monodisperse” sample which is considered uniform in size, a PDI of ≤0.1 is required. Any mixture of lipid vesicle particles with a PDI>0.1 is considered “polydisperse” and is not uniform in size.

In an embodiment, the non-sized lipid vesicle particles may be of any size within the range of 2 nm to 5 μm, or larger. With respect to any mixture of non-sized lipid vesicle particles, the mixture may comprise lipid vesicle particles of any number of different sizes within the range of 2 nm to 5 μm, or larger (i.e. any distribution of size). Also, the mean particle size of the non-sized lipid vesicle particles may be of any size within the range of 2 nm to 5 μm, or larger.

As used herein, “mean” refers to the arithmetic mean of the particle size of the lipid vesicle particles in a given population. It is a synonym for average. As such, “mean particle size” is intended to refer to the sum of the diameters of each lipid vesicle particle of a population, divided by the total number of lipid vesicle particles in the population (e.g. in a population with 4 lipid vesicle particles with particle sizes of 95 nm, 98 nm, 102 nm and 99 nm, the mean particle size is (95+98+102+99)/4=98.5 nm). However, as the skilled person will appreciate, lipid vesicle particles may not be perfectly spherical, and therefore the “particle size” of a given vesicle particle may not be an exact measure of its diameter. Rather, the particle size may be defined by other means known in the art, including for example: the diameter of the sphere of equal area or the largest perpendicular distance between parallel tangents touching opposite sides of the particle (Feret's statistical diameter).

There are several techniques, instruments and services that are available to measure the mean particle size of lipid vesicle particles, such as electron microscopy (transmission, TEM, or scanning, SEM), atomic force microscopy (AFM), Fourier-transform infrared spectroscopy (FTIR), X-ray photoelectron spectroscopy (XPS), powder X-ray diffraction (XRD), matrix-assisted laser desorption/ionization time-of-flight mass spectrometry (MALDI-TOF-MS), nuclear magnetic resonance (NMR) and dynamic light scattering (DLS). DLS is a well-established technique for measuring the particle size in the submicron size range, with available technology to measure particle sizes of less than 1 nm (LS Instruments, CH; Malvern Instruments, UK).

In an embodiment, the non-sized lipid vesicle particles have a mean particle size of any size within the range of 2 nm to 5 μm, or larger. In an embodiment, the non-sized lipid vesicle particles have a mean particle size of >120 nm. In an embodiment, the non-sized lipid vesicle particles have a mean particle size within the range 3 μm to 5 μm. In an embodiment, the non-sized lipid vesicle particles have a PDI of >0.1.

Although it is described above how the mean particle size and PDI of the non-sized lipid vesicle particles may be determined, it is not necessary in the practice of the methods disclosed herein to determine, control or monitor the size and PDI of the non-sized lipid vesicle particles. The non-sized lipid vesicle particles may be of any size and of any distribution of size.

Procedures for preparing lipid vesicle particles are well known in the art. In an embodiment, standard procedures for preparing lipid vesicle particles of any size may be employed. For example, conventional liposome forming processes may be used, such as the hydration of solvent-solubilized lipids. Exemplary methods of preparing liposomes are discussed, for example, in Gregoriadis 1990; and Frezard 1999.

In an embodiment of the disclosed methods, to provide a non-sized lipid vesicle particle preparation, lipids in dry powder form may be added to a solution containing one or more solubilized first therapeutic agents. In such embodiments, the non-sized lipid vesicle particles are formed in the presence of the one or more first therapeutic agents to provide the non-sized lipid vesicle particle preparation. In another embodiment, lipids in dry powder form may be combined with one or more dry first therapeutic agents, and the dry combination may be solubilized together in an appropriate solvent. These embodiments may be performed with shaking and/or mixing (e.g. at 300 RPM for about 1 hour).

In another embodiment of the methods disclosed herein, to provide a non-sized lipid vesicle particle preparation, lipids may first be dissolved and mixed in an organic solvent. In embodiments where different types of lipid are used, this step will allow a homogenous mixture of the lipids to be formed. In an embodiment, these steps may be carried out in chloroform, chloroform:methanol mixtures, tertiary butanol or cyclohexane. In an embodiment, the lipids are prepared at 10-20 mg lipid/mL organic solvent; however, higher or lower concentrations may also be used. After mixing, the organic solvent is removed (e.g. by evaporation) to yield a lipid film. The lipid film may then be frozen and lyophilized to yield a dry lipid film. The dry lipid film may then be hydrated with an aqueous solution containing one or more of the solubilized first therapeutic agents to provide the non-sized lipid vesicle particle preparation. The step of hydration may be performed with shaking and/or mixing (e.g. at 300 RPM for about 1 hour).

In yet another embodiment of the methods disclosed herein, to provide a non-sized lipid vesicle particle preparation, an aqueous solution of lipids may be combined with a solution containing one or more solubilized first therapeutic agents. In another embodiment, one or more dry first therapeutic agents may be added to, and solubilized in, the aqueous solution of lipids to provide a non-sized lipid vesicle preparation. These embodiments may be performed with shaking and/or mixing (e.g. at 300 RPM for about 1 hour).

The above procedures are exemplary methods for providing a non-sized lipid vesicle particle preparation comprising non-sized lipid vesicles and one or more first therapeutic agents. The skilled person will recognize that other protocols may be used, and that the non-sized lipid vesicle preparation may be prepared using any acceptable combination of the above protocols and/or other protocols known in the art.

In an embodiment, during the preparation of the non-sized lipid vesicle particle preparation, at least some of one or more of the first therapeutic agents is encapsulated in the non-sized lipid vesicle particles. In an embodiment, all or a majority of one or more of the first therapeutic agents is encapsulated in the non-sized lipid vesicle particles.

In an embodiment, during the preparation of the non-sized lipid vesicle particle preparation, at least some of each of the first therapeutic agents used is encapsulated in the non-sized lipid vesicle particles. In an embodiment, all or a majority of each of the first therapeutic agents used is encapsulated in the non-sized lipid vesicle particles.

In an embodiment, prior to the step of sizing the non-sized lipid vesicle particle preparation, the non-sized lipid vesicle particle preparation is mixed to disintegrate the lipids. This step may be performed, for example and without limitation, by mixing at 3000 rpm for a period of 15-45 minutes or by mixing with glass beads on a shaker. In an embodiment, this mixing step is performed during the preparation of the non-sized lipid vesicle particles in the presence of the one or more first therapeutic agents (e.g. in the protocols described above). In an embodiment, this mixing step is performed after the non-sized lipid vesicle particle preparation is prepared, just prior to sizing. In an embodiment, this mixing step is performed both during the preparation of the non-sized lipid vesicle particles in the presence of the one or more first therapeutic agents and immediately prior to sizing. In an embodiment, the mixing is performed using a Silverson AX60 high speed mixer.

In an embodiment, throughout the process of preparing the non-sized lipid vesicle particle preparation, the pH is maintained at 9.5±1.0. In an embodiment, just prior to the step of sizing the non-sized lipid vesicle particle preparation, the pH is adjusted to 10.0±0.5. Depending on the lipids, first therapeutic agents and/or solvents that are used, it may be appropriate to make adjustments to these exemplary pH values.

In an embodiment, step (a) of the disclosed methods comprises (a1) providing a therapeutic agent stock comprising the at least one solubilized first therapeutic agent, and optionally further comprising a solubilized adjuvant; and (a2) mixing the therapeutic agent stock with a lipid mixture to form the non-sized lipid vesicle preparation. As used herein, the “lipid mixture” may be a mixture of a single type of lipid (e.g. DOPC only) or it may be a mixture of any two or more different types of lipids (e.g. DOPC and cholesterol). The lipid mixture may be provided as a dry powder mixture, a dry lipid film mixture or a mixture in solution.

In an embodiment, the therapeutic agent stock may be prepared with a single solubilized first therapeutic agent. In another embodiment, involving multiple different first therapeutic agents, the therapeutic agent stock may be prepared by combining individual stock preparations of different solubilized first therapeutics agents. These individual stock preparations may each comprise one or more different first therapeutic agents. In an embodiment, each individual stock preparation comprises a single first therapeutic agent, all of which are then combined to form, in whole or in part, to form the therapeutic agent stock.

In another embodiment, the therapeutic agent stock may be prepared by combining dry first therapeutic agents, adding a solvent and mixing the first therapeutic agents in the solvent. In another embodiment, the therapeutic agent stock may be prepared by combining one or more dry powder first therapeutic agents with one or more solubilized first therapeutic agents.

In an embodiment, the therapeutic agent stock is prepared by sequentially adding individual stock preparations, each comprising one or more different first therapeutic agents, into a compatible solvent with mixing. By “compatible” it is meant that the solvent will not cause the solubilized first therapeutic agents to come out of solution.

The skilled person will appreciate that there are many suitable ways in which a therapeutic agent stock comprising one or more solubilized first therapeutic agents can be prepared. The above procedures are exemplary, without limitation.

The mixing of the therapeutic agent stock and the lipid mixture may be performed by any suitable means. In an embodiment, the mixing is by shaking and/or mixing at 300 RPM for about 1 hour. In an embodiment, the mixing is performed using a Silverson AX60 high speed mixer (e.g. at 3000 rpm for a period of 15-45 minutes).

In accordance with the disclosed methods, in step (a) the non-sized lipid vesicle particle preparation comprises non-sized lipid vesicle particles and at least one solubilized first therapeutic agent. In an embodiment, the at least one first therapeutic agent is a single first therapeutic agent. In another embodiment, the at least one first therapeutic agent is 2, 3, 4, 5, 6, 7, 8, 9, 10 or more different first therapeutic agents. In an embodiment, the at least one first therapeutic agent is 2 to 10 different therapeutic agents. In an embodiment, the at least one first therapeutic agent is 2, 3, 4 or 5 different first therapeutic agents. In a particular embodiment, the at least one first therapeutic agent is four different first therapeutic agents.

In an embodiment, the at least one solubilized first therapeutic agent is a molecule, substance or compound that is soluble at alkaline pH (i.e. pH>7) during the membrane size extrusion procedures as described herein. For example, in an embodiment, the at least one solubilized first therapeutic agent is soluble at alkaline pH during high pressure membrane extrusion with a 0.2 μm membrane, 0.1 μm membrane and/or 0.08 μm membrane, such as when the extrusion is performed at 1000-5000 psi back pressure, or more particularly at about 5000 psi.

In a particular embodiment of the methods disclosed herein, the at least one solubilized first therapeutic agent is one or more peptide antigens as described herein. In a particular aspect of such embodiments, the at least one solubilized first therapeutic agent may be four different peptide antigens, such as for example: FTELTLGEF (SEQ ID NO: 1); LMLGEFLKL (SEQ ID NO: 2); STFKNWPFL (SEQ ID NO: 3); and LPPAWQPFL (SEQ ID NO: 4).

As used herein, by “solubilized first therapeutic agent”, it is meant that the first therapeutic agents are dissolved in a solvent. In an embodiment, this may be determined visually by the naked eye by observing a clear solution. A hazy solution is indicative of insolubility and is not desired for the methods disclosed herein as it may be problematic to forming a clear composition when the dried lipid/therapeutic agent preparation is subsequently solubilized in the hydrophobic carrier.

As described herein, the disclosed methods are advantageous in preparing stable, water-free compositions comprising lipids and therapeutic agents. To prepare such compositions, there are complex formulation requirements. The solvents used in the preparation the non-sized lipid vesicle particle/therapeutic agent mixture must not only be suitable for solubilizing the therapeutic agents in an aqueous environment with the lipids, but must also be suitable for forming a dried lipid/therapeutic agent preparation that will be compatible with a hydrophobic carrier (e.g. any salts and/or non-volatile solvents should preferably be compatible with the hydrophobic carrier). Moreover, the solvent(s) ideally would be suitable for universally solubilizing all of the first therapeutic agents to form the non-sized lipid vesicle preparation.

Through extensive study, the present inventors have identified a number of exemplary solvents which may have broad application in the methods disclosed herein for solubilizing the first therapeutic agents, including optimal salt and/or pH conditions for obtaining a clear pharmaceutical composition.

Exemplary solvents that may be used for solubilizing the first therapeutic agent include, for example and without limitation, zwitterionic solvents. Non-limiting examples of zwitterionic solvents include HEPES (4-(2-hydroxyethyl)-1-piperazineethanesulfonic acid), MOPS (3-(N-Morpholino) propanesulfonic acid) and MES (2-(N-morpholino)ethanesulfonic acid).

In another embodiment, exemplary solvents for solubilizing the first therapeutic agent are aqueous salt solutions. Salts provide useful properties in solubilizing the therapeutic agents, and it has also been recognized that certain salts provide stability to the dried lipid/therapeutic agent preparation. Non-limiting examples of such solvents include sodium acetate, sodium phosphate, sodium carbonate, sodium bicarbonate, potassium acetate, potassium phosphate, potassium carbonate, and potassium bicarbonate.

In an embodiment, the solvent is aqueous sodium acetate. It has been observed in the course of the present invention that sodium acetate imparts favourable properties to the dried lipid/therapeutic agent preparation for subsequent solubilization in the hydrophobic carrier. This is observed over a broad pH range (e.g. 6.0-10.5). For dissolution of multiple different first therapeutic agents, a molarity in the range of 50-200 mM may be preferred.

In an embodiment, the sodium acetate may be 25-250 mM sodium acetate having a pH in the range of 6.0-10.5. In an embodiment, the solvent is 50 mM sodium acetate having a pH of 6.0±1.0. In an embodiment, the solvent is 100 mM sodium acetate having a pH of 9.5±1.0.

In an embodiment, the solvent is 100 mM sodium acetate having a pH of 9.5±0.5.

In an embodiment, the solvent is aqueous sodium phosphate. In an embodiment, the sodium phosphate may be 25-250 mM sodium phosphate having a pH in the range of 6.0-8.0. In an embodiment, the solvent is 50 mM sodium phosphate having a pH of 7.0±1.0. In an embodiment, the solvent is 100 mM sodium phosphate having a pH of 6.0±1.0. In an embodiment, the solvent is 50 mM sodium phosphate having a pH of 7.0. In an embodiment, the solvent is 100 mM sodium phosphate having a pH of 6.0.

Depending on the characteristics of the first therapeutic agent, in certain embodiments it may be advantageous to initially solubilize the first therapeutic agent(s) in a mild/weak acidic solvent (e.g. for basic therapeutic agents) or a mild/weak basic solvent (e.g. for acidic therapeutic agents). Exemplary acidic solvents that may be used include, without limitation, hydrochloric acid, acetic acid. Exemplary basic solvents that may be used include, without limitation, sodium hydroxide, sodium bicarbonate, sodium acetate and sodium carbonate. For neutral therapeutic agents, an exemplary solvent may be dimethyl sulfoxide (DMSO).

In an embodiment, one or more of the first therapeutic agents are initially solubilized in a mild/weak basic solvent. In an embodiment, one or more of the first therapeutic agents are initially solubilized 50-250 mM sodium hydroxide. In an embodiment, the solvent is 200 mM sodium hydroxide.

In the methods disclosed herein, the first therapeutic agents may be solubilized in any of the solvents described herein. Based on the present disclosure, the skilled person could also identify other solvents that may be used that exhibit similar characteristics to those described herein.

In an embodiment, to provide the non-sized lipid vesicle particle preparation the lipids may be combined with the first therapeutic agents in the same or different solvents as are used for solubilizing one or more of the first therapeutic agents. In an embodiment, the non-sized lipid vesicle particle preparation is prepared and provided in a sodium acetate or sodium phosphate solution.

In an embodiment, the non-sized lipid vesicle particle preparation is prepared and provided in 25-250 mM sodium acetate having a pH in the range of 6.0-10.5 or 25-250 mM sodium phosphate having a pH in the range of 6.0-8.0.

In an embodiment, the non-sized lipid vesicle particle preparation is prepared and provided in 50 mM sodium acetate having a pH of 6.0+1.0, 100 mM sodium acetate having a pH of 9.5±1.0, 50 mM sodium phosphate having a pH of 7.0±1.0 or 100 mM sodium phosphate having a pH of 6.0±1.0.

In an embodiment, the non-sized lipid vesicle particle preparation is prepared and provided in 100 mM sodium acetate having a pH of 9.5±1.0.

In an embodiment, after preparation of the non-sized lipid vesicle particle preparation, the pH of the mixture is adjusted to 10±1.0. In an embodiment, the pH is adjusted to 10±0.5.

As encompassed herein, any other optional components (e.g. T-helper epitope and/or adjuvant) may also be solubilized in the solvents described herein to prepare the non-sized lipid vesicle particle preparation.

In an embodiment, at any stage of preparing the solubilized first therapeutic agents or combining the first therapeutic agents with the lipids to for the non-sized lipid vesicle particles, one or more T-helper epitopes and/or adjuvants may be added. The adjuvant and T-helper epitope may be added at any stage and in any order, independent of one another. Typically, embodiments of the methods disclosed herein that involve the use of T-helper epitopes and/or adjuvants are those in which the therapeutic agent comprises at least one peptide antigen or a polynucleotide encoding an antigen.

In an embodiment, during the preparation of the non-sized lipid vesicle particle preparation, one or more T-helper epitopes and/or adjuvants is encapsulated in the non-sized lipid vesicle particles.

Exemplary embodiments of T-helper epitopes and adjuvants that may be used in the practice of the methods disclosed herein are described below, without limitation. In an embodiment, the T-helper epitope comprises or consists of the modified Tetanus toxin peptide A16L (830 to 844; AQYIKANSKFIGITEL; SEQ ID NO: 5). In an embodiment, the adjuvant is a polyI:C nucleotide adjuvant.

In an embodiment, an adjuvant is added during the preparation of the non-sized lipid vesicle particle preparation such that the preparation comprises an adjuvant. In an embodiment, the adjuvant may be provided together with the therapeutic agent stock comprising the first therapeutic agents. Prior to being added to the therapeutic agent stock, the adjuvant may be pre-solubilized in a solvent. In an embodiment, the solvent is water or any other solvent described herein. In an alternative embodiment, the adjuvant is added to the therapeutic stock in a dry form and mixed. In an embodiment, the adjuvant is a polyI:C nucleotide adjuvant.

Step (b) of the disclosed methods involves sizing the non-sized lipid vesicle particle preparation to form a sized lipid vesicle particle preparation comprising sized lipid vesicle particles and said at least one solubilized first therapeutic agent. The methods disclosed herein require sizing of the non-sized lipid vesicle particles to a mean particle size of ≤120 nm and a polydispersity index (PDI) of ≤0.1.

The meaning of “mean particle size” and “polydispersity index (PDI)”, as used herein, has already been described above, together with techniques, instruments and services that are available to measure the mean particle size and PDI.

In an embodiment, the mean particle size of ≤120 is measured by any instrument and/or machine suitable for measuring the mean particle size of lipid vesicle particles, such as by the methods above.

In an embodiment of the methods disclosed herein, the mean particle size is determined by DLS (Malvern Instruments, UK).

In an embodiment, the mean particle size of ≤120 is measured by DLS using a Malvern Zetasizer series instrument, such as for example the Zetasizer Nano S, Zetasizer APS, Zetasizer μV or Zetasizer AT machines (Malvern Instruments, UK). In an embodiment, the mean particle size of ≤120 is measured by DLS using a Malvern Zetasizer Nano S machine. Exemplary conditions and system settings may include:

Dispersant Name: 0.006% NaCl

Dispersant RI: 1.330

Viscosity (cP): 0.8872

Temperature (° C.): 25.0

Duration Used (s): 60

Count Rate (kcps): 200-400

Measurement Position (mm): 4.65

Cell Description: Disposable Sizing Cuvette

Attenuator: 7

The sized lipid vesicle particles have a mean particle size of less than or equal to 120 nanometers (i.e. ≤120 nm) and a PDI of less than or equal to 0.1 (i.e. ≤0.1). In an embodiment, the sized lipid vesicle particles have a mean particle size of ≤115 nm, more particularly still ≤110 nm and more particularly still ≤100 nm. In an embodiment, the mean particle size of the sized lipid vesicle particles is between 50 nm and 120 nm. In an embodiment, the mean particle size of the sized lipid vesicle particles is between 80 nm and 120 nm. In an embodiment, the mean particle size of the sized lipid vesicle particles is between about 80 nm and about 115 nm, about 85 nm and about 115 nm, about 90 nm and about 115 nm, about 95 nm and about 115 nm, about 100 nm and about 115 nm or about 105 nm and about 115 nm.

In an embodiment, the mean particle size of the sized lipid vesicle particles is about 80 nm, about 81 nm, about 82 nm, about 83 nm, about 84 nm, about 85 nm, about 86 nm, about 87 nm, about 88 nm, about 89 nm, about 90 nm, about 91 nm, about 92 nm, about 93 nm, about 94 nm, about 95 nm, about 96 nm, about 97 nm, about 98 nm, about 99 nm, about 100 nm, about 101 nm, about 102 nm, about 103 nm, about 104 nm, about 105 nm, about 106 nm, about 107 nm, about 108 nm, about 109 nm, about 110 nm, about 111 nm, about 112 nm, about 113 nm, about 114 nm, about 115 nm, about 116 nm, about 117 nm, about 118 nm or about 119 nm. In an embodiment, the mean particle size is 120 nm.

As used throughout herein, the term “about” means reasonably close. For example, “about” can mean within an acceptable standard deviation and/or an acceptable error range for the particular value as determined by one of ordinary skill in the art, which will depend on how the particular value is measured. Further, when whole numbers are represented, about can refer to decimal values on either side of the whole number. When used in the context of a range, the term “about” encompasses all of the exemplary values between the one particular value at one end of the range and the other particular value at the other end of the range, as well as reasonably close values beyond each end.

With respect to the mean particle size, the term “about” is used to represent a deviation of ±2.0 nm, so long as it would not cause the mean particle size to exceed 120 nm. Also, the term “about” is meant to encompass any decimal number of the indicated mean particle size.

In an embodiment, the mean particle size of the sized lipid vesicle particles is between about 105 nm and about 115 nm, such as for example when the lipid vesicle particles are formed from DOPC/cholesterol (10:1 w:w).

The PDI of the sized lipid vesicle particles is ≤0.1. In an embodiment, the PDI of ≤0.1 is measured by any instrument and/or machine suitable for measuring the PDI of lipid vesicle particles.

In an embodiment of the PDI size distribution is determined by DLS (Malvern Instruments, UK).

In an embodiment, the PDI of ≤0.1 is measured by DLS using a Malvern Zetasizer series instrument, such as for example the Zetasizer Nano S, Zetasizer APS, Zetasizer μV or Zetasizer AT machines (Malvern Instruments, UK). In an embodiment, the PDI of ≤0.1 is measured by DLS using a Malvern Zetasizer Nano S machine. Exemplary conditions and system settings are described above in respect of determining mean particle size.

The requirement that the sized lipid vesicle particles have a mean particle size of ≤120 nm and a PDI of ≤0.1 means that it is possible that some lipid vesicle particles in a given population will have a particle size that is greater than 120 nm. This is acceptable so long as the mean particle size remains ≤120 nm and the PDI remains ≤0.1. As is shown in Example 1, lipid vesicle particles that are sized to meet these specifications are advantageous over non-sized lipid vesicle particles in obtaining a suitable dried lipid/therapeutic agent preparation for subsequent solubilization in a hydrophobic carrier (i.e. in obtaining a clear solution).

There are various techniques available in the art for sizing lipid vesicle particles (see e.g. Akbarzadeh 2013). For example, in an embodiment, the non-sized lipid vesicle particle preparation may be sized by high pressure homogenization (microfluidizers), sonication or membrane based extrusion.

In an embodiment, the sizing of the non-sized lipid vesicle particle preparation is performed using membrane based extrusion to obtain the sized lipid vesicle particles having a mean particle size of ≤120 nm and a PDI of ≤0.1. Exemplary, non-limiting embodiments of membrane based extrusion include passing the non-sized lipid vesicle particle preparation through a 0.2 μm polycarbonate membrane and then through a 0.1 μm polycarbonate membrane, and then optionally through a 0.08 μm polycarbonate membrane. Exemplary, non-limiting protocols may include: (i) passing the non-sized lipid vesicle particle preparation 20-40 times through a 0.2 μm polycarbonate membrane, and then 10-20 times through a 0.1 μm polycarbonate membrane; or (ii) passing the non-sized lipid vesicle particle preparation 20-40 times through a 0.2 μm polycarbonate membrane, then 10-20 times through a 0.1 μm polycarbonate membrane, and then 10-20 times through a 0.08 μm polycarbonate membrane. The skilled would be well aware of different membranes and different protocols which may be used to attain the required mean particle size of ≤120 nm and PDI of ≤0.1.

In a particular embodiment, the sizing may be performed by passing a non-sized lipid vesicle particle preparation 25 times through a 0.2 μm polycarbonate membrane, and then 10 times through a 0.1 μm polycarbonate membrane. In another particular embodiment, the sizing may be performed by passing a non-sized lipid vesicle particle preparation 25 times through a 0.2 μm polycarbonate membrane, then 10 times through a 0.1 μm polycarbonate membrane, and then 15 times through a 0.08 μm polycarbonate membrane.

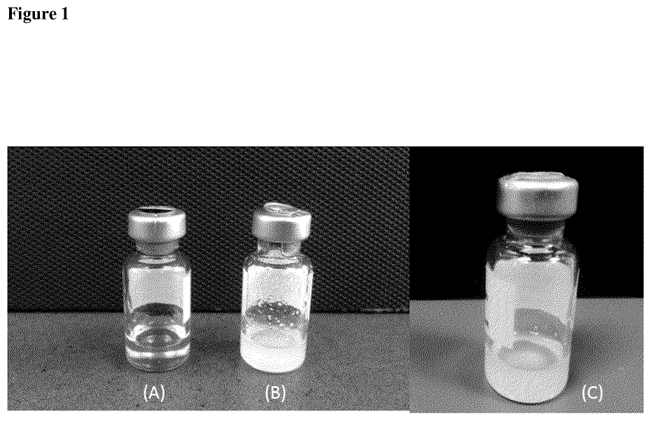

It has been found that sizing of the lipid vesicle particles to a mean particle size of ≤120 nm and PDI of ≤0.1 is an advantageous property. As shown in Example 1, a non-sized lipid vesicle preparation resulted in a turbid composition (FIG. 1C). This is indicative of components of the composition having precipitated out during the manufacturing process (e.g. precipitation of therapeutic agents during sterile filtration) and/or an incompatibility of components with one or more of the aqueous and/or hydrophobic phases. Indeed, as shown in Table 6, compositions prepared with non-sized lipid vesicle particles result in a low percent solubilization of the therapeutic agents in the hydrophobic carrier (i.e. 16-35% solubility). In contrast, when the lipid vesicle particles are sized to a mean particle size of ≤120 nm and PDI of ≤0.1, a clear solution is obtained (FIG. 1A) and the percent solubility of the therapeutic agents is significantly increased (i.e. >98%; Table 6).

The membrane extrusion is typically performed under high back pressure. In an embodiment, the membrane extrusion is performed at 1000 to 5000 psi back pressure. Under these conditions, during the size extrusion process a back pressure of above 5000 psi may signal an issue with the solubility of one or more of the first therapeutic agents.

It has been found by the present inventors during manufacturing process development that certain therapeutic agents, such as positively charged hydrophobic agents, encounter precipitation issues upon size extrusion. Despite being capable of solubilization during formation of the non-sized lipid vesicle particle preparation, the conditions of the size extrusion cause certain therapeutic agents to precipitate out. This is a problematic feature for preparing pharmaceutical-grade compositions involving lipid vesicle delivery systems and hydrophobic carriers.

Unexpectedly, it has been found that by adding one or more of the therapeutic agents after sizing of the lipid vesicle particles, it is possible to avoid precipitation of therapeutic agents and still obtain stable, clear, water-free pharmaceutical compositions with a significantly high percent solubilization of therapeutic agents (FIG. 1A; Table 6). This is an advantageous property in that the second therapeutic agents are still able to withstand (e.g. not precipitate out) multiple different phases encountered during preparation of the pharmaceutical composition, e.g. aqueous phase, drying and hydrophobic phase, even though they are not present when the lipid vesicle particles are formed. This is not observed with non-sized lipid vesicles whereby a turbid solution with precipitate is observed.

Without being bound by theory, it is believed that the sized lipid vesicle particles may be capable of rearranging to form different structures depending on the processing step (e.g. drying, solubilization in a hydrophobic carrier, etc.). The small and uniform size of the sized lipid vesicle particles (i.e. mean particle size 120 nm with a PDI 0.1) may make them particularly amenable to these conformation changes. For example, when placed in a hydrophobic carrier, the sized lipid vesicle particles may reorder to form alternate lipid-based structures as described herein. Indeed, it is believed that a rearrangement of the lipids occurs during these subsequent manufacturing steps, as shown for example by the SAXS analysis provided herein.

In this regard, step (c) of the disclosed methods involves mixing the sized lipid vesicle particle preparation with at least one second therapeutic agent to form a mixture.

The second therapeutic agent may be any of the therapeutic agents as described herein. In an embodiment, the second therapeutic agent is one that is not compatible with size extrusion procedures (e.g. precipitates under high pressure extrusion). In an embodiment, the second therapeutic agent is one that tends to be stable (e.g. soluble) in acidic or slightly acidic pH and/or unstable (e.g. insoluble) in alkaline or slightly alkaline pH. In a particular embodiment, the second therapeutic agent(s) are short, positively charged hydrophobic peptides, such as for example peptides of 5-40 amino acids in length, more particularly 5-20 amino acids in length, and more particularly still 5-10 amino acids in length.

In an embodiment of the disclosed methods, the at least one second therapeutic agent is a single second therapeutic agent. In another embodiment, the at least one second therapeutic agent is 2, 3, 4, 5, 6, 7, 8, 9 or 10 different second therapeutic agents. In an embodiment, the at least one second therapeutic agent is 2, 3, 4 or 5 different second therapeutic agents.

In a particular embodiment of the methods disclosed herein, the at least one second therapeutic agent is one or more peptide antigens as described herein. In a particular aspect of such embodiments, the second therapeutic agent is a single peptide antigen having the amino acid sequence RISTFKNWPK (SEQ ID NO: 6).

The one or more second therapeutic agents are either solubilized in a solvent prior to mixing with the sized lipid vesicle particle preparation or the one or more second therapeutic agents are solubilized upon being mixed with the sized lipid vesicle particle preparation. In this latter embodiment, the second therapeutic agents may be added as a dry powder to a solution containing the sized lipid vesicle particle preparation or both the sized lipid vesicle particle preparation and dry second therapeutic agents may be mixed together in a fresh solvent.

When the therapeutic agents are solubilized prior to mixing with the sized lipid vesicle particle preparation, in embodiments where more than one second therapeutic agent is used, the individual second therapeutic agents may be solubilized together in the same solvent or separate from each other in different solvents. When three or more therapeutic agents are used, some of the agents may be solubilized together and others may be solubilized individually.

In an embodiment, each of the second therapeutic agents are solubilized separately as therapeutic agent stocks, and added sequentially to the sized lipid vesicle particle preparation.

The solvent for solubilizing the second therapeutic agent may be one or more of the same solvents described herein for solubilizing the first therapeutic agent. Based on the present disclosure, the skilled person could also identify other solvents that may be used that exhibit similar characteristics to those described herein.

In an embodiment, the one or more second therapeutic agents are solubilized in a mild acid. Without limitation, the mild acid may for example be mild acetic acid. In an embodiment, the one or more second therapeutic agents are solubilized in a 0.1-0.5% (w/w) acetic acid solution, more particularly a 0.25% (w/w) acetic acid solution.

Similar to the first therapeutic agents, as used herein “solubilized” with respect to the second therapeutic agents means that the second therapeutic agents are dissolved in a solvent. In an embodiment, this may be determined visually by the naked eye by observing a clear solution. A hazy solution is indicative of insolubility and is not desired for the methods disclosed herein as it may be problematic to forming a clear composition when the dried lipid/therapeutic agent preparation is subsequently solubilized in the hydrophobic carrier.

As encompassed herein, in step (c) of the disclosed methods, other optional components (e.g. T-helper epitope and/or adjuvant) may also be mixed with the sized lipid vesicle particle preparation.

In an embodiment, at any stage of preparing the solubilized second therapeutic agents or mixing the second therapeutic agents with the sized lipid vesicle particle preparation, one or more T-helper epitopes and/or adjuvants may be added. The adjuvant and T-helper epitope may be added at any stage and in any order, independent of one another. Typically, embodiments of the methods disclosed herein that involve the use of T-helper epitopes and/or adjuvants are those in which the therapeutic agent comprises at least one peptide antigen or a polynucleotide encoding an antigen.

In a particular embodiment of the methods disclosed herein, step (c) further comprises mixing a T-helper epitope with the sized lipid vesicle particle preparation and the at least one second therapeutic agent. In an embodiment, the T-helper epitope comprises or consists of the modified Tetanus toxin peptide A16L (830 to 844; AQYIKANSKFIGITEL; SEQ ID NO: 5).

In an embodiment, the T-helper epitope may be prepared as an individual stock, solubilized in a suitable solvent. In an embodiment, the solvent is a mild acid such as, for example, mild acetic acid (e.g. 0.25% w/w). The T-helper epitope may then be mixed with the sized lipid vesicle particle preparation before, after or concurrently with the one or more second therapeutic agents.

In another embodiment, the T-helper epitope may be provided together in the same solution as the therapeutic agent stock comprising the second therapeutic agents. Prior to being added to the therapeutic agent stock, the T-helper epitope may be pre-solubilized in a solvent, such as for example a mild acid (e.g. 0.25% w/w acetic acid). In an alternative embodiment, the T-helper epitope may be added to the therapeutic stock in a dry form and mixed.

The actual mixing of the sized lipid vesicle particle preparation and the one or more second therapeutic agents (and any other optional components) may be performed under any suitable conditions for obtaining a generally homogenous mixture. However, the mixing should not be performed under aggressive conditions that might cause the sized lipid vesicle particles and/or therapeutic agents to precipitate out of solution. In an embodiment, the mixing may be performed with gentle shaking or stirring at 100-500 RPM for a period of 2-60 minutes. In an embodiment, the mixing may be performed by shaking/stirring at 300 RPM for a period of about 3 minutes. In another embodiment, the mixing may be performed by shaking/stirring at 300 RPM for a period of about 15 minutes.

The mixture formed in step (c) may hereinafter be referred to as a “sized lipid vesicle particle/therapeutic agent mixture”.

In accordance with the disclosed methods, in step (d) the sized lipid vesicle particle/therapeutic agent mixture is dried to form a dried lipid/therapeutic agent preparation.

As used herein, the terms “dried preparation”, “dried lipid/therapeutic agent preparation” or “dried preparation comprising lipids and therapeutic agents”, used interchangeably, do not necessarily mean that the preparation is completely dry. For example, depending on the solvent or solvents used in the methods disclosed herein, it is possible that a small component of volatile and/or non-volatile material will remain in the dried preparation. In an embodiment, the non-volatile material will remain. By “dried preparation”, it is meant that the preparation no longer contains substantial quantities of water. The process used to dry the preparation should be capable of removing substantially all water from the sized lipid vesicle particle/therapeutic agent mixture. Thus, in an embodiment, the dried preparation is completely free of water. In another embodiment, the dried preparation may contain a residual moisture content based on the limitations of the drying process (e.g. lyophilization). This residual moisture content will typically be less than 2%, less than 1%, less than 0.5%, less than 0.25%, less than 0.1%, less than 0.05% or less by weight of the dried preparation. This residual moisture content will not be more than 5% by weight of the dried preparation as this would result in a product that is not clear.

Various methods may be used to dry the sized lipid vesicle particle/therapeutic agent mixture, which are known in the art. In an embodiment, the drying is performed by lyophilization, spray freeze-drying, or spray drying. The skilled person is well-aware of these drying techniques and how they may be performed.

In an embodiment, the drying is performed by lyophilization. As used herein, “lyophilization”, “lyophilized” and “freeze-drying” are used interchangeably. As is well known in the art, lyophilization works by freezing the material and then reducing the surrounding pressure to allow the volatile solvent (e.g. water) in the material to sublime directly from the solid phase to the gas phase.

Any conventional freeze-drying procedure may be used to carry out the drying step of the methods disclosed herein. In an embodiment, the lyophilization is performed by sequential steps of loading, freezing, evacuation and drying (e.g. primary drying and secondary drying).

In an embodiment, the lyophilization is performed according to the protocol set forth in Table 3 below (Example 1). Briefly, the mixture of sized lipid vesicle particles and therapeutic agent is frozen to a temperature of about −50° C. Evacuation is then performed by reducing the pressure to about 100 micron (mTorr). The mixture is then dried. A primary drying is performed for about 55 hours by increasing the temperature to about −40° C. under the reduced pressure. Then, a secondary drying is performed for about 20 minutes by further increasing the temperature to about 35° C. under the reduced pressure.

Relevant considerations for the freezing and drying stages include:
Freezing: It is important to cool the material below its triple point, i.e. the lowest temperature at which the solid and liquid phases of the material can coexist. This ensures that sublimation rather than melting will occur in the following steps.
Primary Drying: Enough heat is supplied for sublimation to occur. This phase may be performed slowly (hours to days). If too much heat is added, the material's structure could be altered.
Secondary Drying: Aims to remove any unfrozen water molecules. The temperature is raised (usually above 0° C.) to break any physico-chemical interactions that have formed between the water molecules and the frozen material.

In an embodiment, lyophilization of the sized lipid vesicle particle/therapeutic agent mixture can be performed within a sealed bag in a benchtop freeze dryer. This may be particularly advantageous because it reduces the number of steps that must be performed in a sterile laboratory environment and allows for the rapid manufacture of smaller batch sizes. For example, after sterile filtration of the sized lipid vesicle particle/therapeutic agent mixture, aseptically filled vials containing the mixture can be loaded and sealed within a sterile bag under sterile conditions. These sterile, sealed units can then undergo lyophilization in an open laboratory (i.e. non-sterile environment) using a benchtop freeze dryer. By this method, it is also possible to perform the freeze-drying with multiple different sealed units in a single freeze dryer. This may reduce the cost and time of manufacture by avoiding expensive freeze-drying steps in sterile laboratory environments using large-scale freeze dryers. Also, multiple different small-scale batches of dried lipid/therapeutic agent preparation may be prepared simultaneously in separate sealed sterile bags.

Thus, in an embodiment, the lyophilization is performed by loading one or more containers comprising the mixture of step (c) into a bag, sealing the bag to form a sealed unit, and then lyophilizing the sealed unit in a freeze dryer. In an embodiment, a single sealed unit may be loaded into the freeze dryer for lyophilization. In another embodiment, multiple separate sealed units may be loaded into a single freeze dryer for lyophilization. In an embodiment, the freeze dryer may contain 1, 2, 3, 4, 5, 6, 7, 8, 9, 10 or more different sealed units for the lyophilization.

In embodiments in which multiple separate sealed units are loaded into a single freeze dryer, the sealed units may: (i) each contain the same sized lipid vesicle particle/therapeutic agent mixture as the other sealed units, (ii) each contain a different sized lipid vesicle particle/therapeutic agent mixture than the other sealed units, or (iii) any combination thereof (i.e. some of the sealed units may contain the same sized lipid vesicle particle/therapeutic agent mixture as other sealed units and some sealed units may contain a different sized lipid vesicle particle/therapeutic agent mixture). The difference between the sized lipid vesicle particle/therapeutic agent mixtures in the sealed units may be in respect of the lipids used to prepare the vesicle particles, the first and/or second therapeutic agents included in the mixture and/or any other component. In a particular embodiment, it is the therapeutic agents that are different as between the sealed units. For the manufacture of pharmaceutical grade compositions, each individual sealed unit should only comprise containers with the same sized lipid vesicle particle/therapeutic agent mixture.

For ease of handling, the containers may be loaded onto a tray and the tray then sealed within the bag. In an embodiment, the tray is a metal tray or a plastic tray.

The container comprising the sized lipid vesicle particle/therapeutic agent mixture may be any container suitable for lyophilization. In an embodiment, the container is a vial, bottle, flask, test tube or any suitable alternative. In an embodiment, the container is a vial, such as a glass or a plastic vial. In an embodiment, the vial is a glass vial. In an embodiment, the container is a 2 mL or 3 mL glass vial, such as for example a 2 mL or 3 mL 13MM FTN BB LYO PF vial. The container may further comprise a stopper and/or a seal suitable for lyophilization. In an embodiment, the stopper is a vented stopper. In an embodiment, the stopper is a Fluorotec Lyophilization Closure, 13MM, single vented stopper. In an embodiment, the seal is crimp seal, such as for example an aluminum crimp seal. In an embodiment, the seal is a West-Spectra Flip-Off 13MM seal.

The bag containing the sample for lyophilization may be any bag that is suitable for lyophilization. In an embodiment, the bag should also be capable of being autoclaved to provide a sterile bag. To provide a sterile bag, the bag is autoclaved and subsequently maintained under sterile conditions. Thus, in an embodiment, the bag is a sterile, autoclaved bag.

In an embodiment, the bag is made of paper, plastic or a paper/plastic combination. In an embodiment, the paper is a medical-grade paper and the plastic is a polyester/polypropylene laminate film. Various types of bags suitable for sterilizing medical equipment are known in the art, and any of these bags may be used. In an embodiment, the sterile bag is a Fisherbrand™ Instant Sealing Sterilization Pouch (Fisher Scientific).

The lyophilization may be performed in any suitable freeze dryer. In an embodiment, the freeze dryer is a benchtop freeze dryer. In an embodiment, the freeze dryer is a Virtis benchtop lyophilizer. In an embodiment, the freeze dryer is in an open laboratory (i.e. non-sterile environment).

The methods disclosed herein for preparing a dried lipid/therapeutic agent preparation may further comprise a step of sterilization. Sterilization may be performed by any method known in the art. In an embodiment, the sterilization is performed by sterile filtration, steam heat sterilization, irradiation (e.g. gamma irradiation) or chemical sterilization. In a particular embodiment, the sterilization is performed by sterile filtration. In an embodiment, the sterile filtration may be performed between steps (c) and (d), i.e. after mixing the sized lipid vesicle particle preparation with the at least one second therapeutic agent, but before drying.

Any conventional procedure for sterile filtration may be employed so long as it does not affect the solubility and stability of the therapeutic agents in the sized lipid vesicle particle/therapeutic agent mixture. In this regard, it may be desirable to perform the sterile filtration under low pressure conditions (e.g. between 30-50 psi).

The serial filtration may be performed using commercially available sterile filtration membranes (e.g. MilliporeSigma). In an embodiment, the sterile filtration is performed using a 0.22 μm-rated membrane, a 0.2 μm-rated membrane and/or a 0.1 μm-rated membrane. In an embodiment, the sterile filtration is performed by a single passage of the sized lipid vesicle particle/therapeutic agent mixture through a single filtration membrane. In another embodiment, the sterile filtration is performed by serially passing the sized lipid vesicle particle/therapeutic agent mixture sequentially through a combination of the same or different sized filtration membranes.

Without limitation, in an embodiment, the sterile filtration may be performed under the following conditions:
1) Filtration pressure: 30-50 psi nitrogen gas
2) Temperature: Room temperature
3) Product Contact Time: ≤45 minutes
4) Filter Type: Millipak-20 PVDF Filter, 0.22 μm
5) Size: 6 L batch size

In an embodiment, the sterile filtration is performed by passing the mixture of step (c) through a single Millipak-20 PVDF Filter, 0.22 μm. In another embodiment, the sterile filtration is performed by serially passing the mixture of step (c) through two or more sterile filtration membranes. In an embodiment of the serial sterile filtration, the mixture of step (c) is passed through two, three, four, five or more Millipak-20 PVDF 0.22 μm membranes. In an embodiment of the serial sterile filtration, the mixture of step (c) is passed through two Millipak-20 PVDF 0.22 μm membranes.

The methods disclosed herein for preparing a dried lipid/therapeutic agent preparation may further comprise a step of confirming that the sized lipid vesicle particles have retained a mean particle size of ≤120 nm and PDI of ≤0.1. As described elsewhere herein, there are several techniques, instruments and services that are available to measure the mean particle size and PDI of lipid vesicle particles, such as for example and without limitation TEM, SEM, AFM, FTIR, XPS, XRD, MALDI-TOF-MS, NMR and DLS.

In an embodiment, the step of confirming the size and PDI of the lipid vesicle particles is performed using a DLS ZETASIZER NANO-S particle size analyzer.

The step of size/PDI confirmation may be performed once or at multiple different times throughout the disclosed methods. In an embodiment, this step may be performed before mixing the sized lipid vesicle particles with the second therapeutic agent in step (c); after mixing the sized lipid vesicle particles with the second therapeutic agent in step (c); and/or before performing the drying of step (d). In an embodiment, the size confirmation step is performed between steps (c) and (d) to confirm the size/PDI of the sized lipid vesicle particles before drying.

In an embodiment, the size confirmation step may be performed by analyzing a small sample volume of a preparation of interest. In another embodiment, the size confirmation step may be performed by analyzing a sample from a preparation that was prepared in parallel with a preparation of interest.

In an embodiment, the step of confirming the size/PDI of the sized lipid vesicle particles also comprises confirming the pH of the sized lipid vesicle particle/therapeutic agent preparation. In an embodiment, the pH is measured using the same machine that is used to measure the size/PDI of the lipid vesicle particles. In an embodiment, the pH is measured separately using any suitable device for determining pH. Exemplary solvents are discussed elsewhere herein and, in an embodiment, this step involves confirming that the solvent retains the desired pH as described herein. For example, in an embodiment where the lipid vesicle particles are suspended in sodium phosphate, this step involves confirming a pH of 6.0-8.0. In an embodiment where the lipid vesicle particles are suspended in sodium acetate, this step involves confirming a pH of 6.0-10.5. More specific exemplary pH values for these solvents, based on molarity, are described elsewhere herein.

The methods disclosed herein for preparing a dried lipid/therapeutic agent preparation may further comprise a step of evaluating the stability of the lipids, therapeutic agent(s) and other components (e.g. adjuvant and T-helper epitope) before and/or after the drying of step (d). The stability of the components may be measured by any known means or method. For example and without limitation, stability of the dried preparation may be determined by the appearance of the dried preparation (lyophilisate) or measurement of the content of the components over time (e.g. by HPLC, RP-HPLC, IEX-HPLC, etc.). HPLC is a technique which can be used to separate, identify and quantify each component in a mixture. Thus, by using HPLC, RP-HPLC or IEX-HPLC it is possible to determine the approximate quantity of the lipids, therapeutic agents and other components, as well as characterize the components qualitatively (e.g. observe impurities, degradation products, etc.).

In other embodiments, stability may evaluated upon solubilization in a hydrophobic carrier by various methods, such as for example: appearance of the solubilized product; identification and quantification of lipids, therapeutic agents and/or other components, impurities or degradants (e.g. by RP-HPLC, IEX-HPLC, etc.); particle size of the lipid-based structures having a single layer lipid assembly (e.g. by SAXS); optical density; viscosity (e.g. as per Ph.Eur. 2.2.9); pH; extractable volume, such as from a syringe (e.g. as per Ph.Eur. 2.9.17), and immunogenicity assays (e.g. ELISpot).

In an embodiment, the methods disclosed herein are capable of providing a sized lipid vesicle particle/therapeutic agent mixture in which at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% of the original amount/concentration of lipids and/or therapeutic agents is retained in undegraded form immediately before drying. In an embodiment, 100% of the original amount/concentration of lipids and/or therapeutic agents is retained in undegraded form immediately before drying.

In an embodiment, the methods disclosed herein are capable of providing a dried lipid/therapeutic agent preparation in which at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% of the original amount/concentration of lipids and/or therapeutic agents is retained in undegraded form immediately after drying. In an embodiment, 100% of the original amount/concentration of lipids and/or therapeutic agents is retained in undegraded form immediately after drying. In an embodiment, the lipid and therapeutic agent content may be measured by solubilizing the dried preparation in a hydrophobic carrier and then performing RP-HPLC.

In an embodiment, the methods disclosed herein are capable of providing a dried lipid/therapeutic agent preparation in which at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% of the original amount/concentration of lipids and/or therapeutic agents is retained in undegraded form at least 3 months, at least 6 months, at least 9 months, at least 12 months, at least 18 months after drying. In an embodiment, 100% of the original amount/concentration of lipids and/or therapeutic agents is retained in undegraded form at least three months after drying. In an embodiment, the lipid and therapeutic agent content may be measured by solubilizing the dried preparation in a hydrophobic carrier and then performing RP-HPLC.

As described later herein, and as shown in Example 6 (Tables 10 and 11), the dried lipid/therapeutic agent preparation prepared in accordance with the disclosed methods using sized lipid vesicle particles having a mean particle size of ≤120 nm and PDI of ≤0.1 exhibit long term stability, including in respect of therapeutic agent added after formation and sizing of the lipid vesicle particles.

In an embodiment of the methods disclosed herein, after step (c), each of the solubilized first and second therapeutic agents is at a concentration of between about 0.1 mg/mL and 10 mg/mL in the sized lipid particle/therapeutic agent mixture. In an embodiment, each of the solubilized first and second therapeutic agents is at a concentration of at least about 0.5 mg/mL, 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL or 2.0 mg/mL. In an embodiment, the therapeutic agents are peptide antigens.

In an embodiment, the methods involve the use of five or more different therapeutic agents and after step (c), each of the different solubilized first and second therapeutic agents is at a concentration of about 1.0 mg/mL. In an embodiment, the therapeutic agents are peptide antigens.

In a particular embodiment of the methods disclosed herein, the therapeutic agents are peptide antigens. For example, the methods disclosed herein may be used in the preparation of peptide-based immunogenic compositions (e.g. vaccines).

Conventional vaccine strategies using whole organisms or large proteins have been highly efficacious for several decades, particularly in the treatment of infectious disease. However, the inclusion of unnecessary antigenic material is problematic in that it often gives rise to undesired reactivity, with protective immunity being dependent upon only a few select peptide epitopes within the formulation. This has created significant interest in peptide-based vaccines.

Fully synthetic peptide-based vaccines are the potential future of vaccination. Peptide vaccines rely on the usage of short peptide fragments to induce highly targeted immune responses. Peptide-based vaccines offer several advantages over conventional vaccines. For example, peptide antigens are less likely to induce undesired allergic or autoimmune responses due to the lack of unnecessary elements; chemical synthesis practically removes all of the problems associated with biological contamination; and peptides can be customized or multi-peptide approaches employed to target very specific objectives.

However, a drawback to peptide-based vaccination is that due to their relatively small size, peptide antigens are often weakly immunogenic and therefore typically require the assistance of adjuvants and/or an effective delivery system. Peptide antigens can also be difficult to formulate in pharmaceutical compositions, particularly when unique delivery systems are involved.

Although the efficiency in identifying potential epitopes has vastly improved with the aid of sequencing techniques and the creation of computer algorithms (e.g. NetMHC which identify motifs predicted to bind MHC class I and/or MHC class II proteins), these technologies do little to accurately predict the ability to generate stable compositions using peptide antigens. Moreover, while the use of multiple peptide antigens is often desirable to provide broader coverage through antigenic diversity, these types of vaccines are often even more difficult to formulate as stable compositions, particularly in the context of specialized delivery systems that employ unique components, such as lipid-based delivery vehicles and/or hydrophobic carriers. Thus, despite advances, the formulation of suitable antigens has remained a crucial and time consuming step in the development of peptide-based vaccines.

In an embodiment, the present disclosure relates to advantageous methods for preparing dried peptide antigen preparations and pharmaceutical compositions comprising peptide antigens. In an embodiment, the present disclosure relates to a method for preparing a dried peptide antigen preparation, said method comprising the steps of: (a) providing a lipid vesicle particle preparation comprising lipid vesicle particles and at least one solubilized peptide antigen; (b) sizing the lipid vesicle particle preparation to form a sized lipid vesicle particle preparation comprising sized lipid vesicle particles and said at least one solubilized peptide antigen, said sized lipid vesicle particles having a mean particle size of ≤120 nm and a polydispersity index (PDI) of ≤0.1; (c) mixing the sized lipid vesicle particle preparation with at least one second peptide antigen to form a mixture, wherein said at least one second peptide antigen is solubilized in the mixture and is different from said at least one solubilized first peptide antigen; and (d) drying the mixture formed in step (c) to form a dried preparation comprising lipids and therapeutic agents.

As disclosed herein, it has been found that by adding one or more of the peptide antigens after formation and sizing of the lipid vesicle particles, it is possible to avoid precipitation of peptide antigen due to high pressure extrusion and still obtain stable, clear, water-free pharmaceutical compositions with a significantly high percent solubilization of peptide antigens (FIG. 1A; Table 6). Without being bound by theory, it is believed that upon mixing with the antigen and/or during subsequent drying (e.g. lyophilization), the small uniformly sized lipid vesicle particles are capable of rearranging themselves (e.g. reordering and/or fusing). The rearrangement of the sized lipid vesicle particle structures may serve to effectively surround the subsequently added peptide antigens in incompatible environments, e.g. hydrophobic peptides in an aqueous environment and then hydrophilic peptides in the hydrophobic carrier. In essence, it is believed that the sized lipid vesicle particles permit rearrangements that allow the peptide antigen payload to properly present to both the hydrophilic and hydrophobic environments. This was not observed with non-sized lipid vesicle particles whereby a dense turbid solution was obtained (FIG. 1C). Likewise, compositions prepared without lipids also resulted in a dense turbid solution (FIG. 1B).

Method for Preparing a Pharmaceutical Composition

In an embodiment, the present invention relates to a method for preparing a pharmaceutical composition. In an embodiment, the pharmaceutical composition is prepared by first preparing a dried lipid/therapeutic agent preparation according to the methods disclosed herein, and then solubilizing the dried preparation in a hydrophobic carrier.

As used herein, by “solubilizing” it is meant that the dried lipid/therapeutic agent preparation is restored to a liquid state by dissolving the dried constituents in a hydrophobic carrier. The hydrophobic carrier may be added by any means that will dissolve the dried constituents (e.g. the lipid and therapeutic agent) in the hydrophobic carrier. For example, and without limitation, the dried lipid/therapeutic agent preparation may be solubilized in the hydrophobic carrier by mixing of the two together. In an embodiment, solubilizing involves adding the hydrophobic carrier to the dried lipid/therapeutic agent preparation, allowing it to sit for 1-30 minutes, and then gently shaking or mixing the mixture for 1-15 minutes. This process can be repeated until the dried constituents are dissolved in the hydrophobic carrier (e.g. a clear solution is obtained).

In an embodiment, solubilizing involves adding the hydrophobic carrier to the dried lipid/therapeutic agent preparation, allowing it to sit for 5 minutes, and then gently shaking or mixing for 1 minute. This process can be repeated until the dried constituents are dissolved in the hydrophobic carrier (e.g. a clear solution is obtained).

In an embodiment, the step of solubilizing the dried lipid/therapeutic agent in a hydrophobic carrier results in a composition in which the dried constituents are fully dissolved in the hydrophobic carrier. In an embodiment, the dried constituents may not be completely dissolved in the hydrophobic carrier, but they are dissolved to a sufficient degree to reproducibly provide a clear solution.

As shown in FIG. 1A, a dried lipid/therapeutic agent preparation produced by the methods disclosed herein is capable of generating a clear solution upon solubilization in a hydrophobic carrier. In contrast, when the dried lipid/therapeutic agent preparation is prepared with non-sized lipid vesicle particles, a dense turbid solution was formed (see FIG. 1C). Likewise, when no lipids were used, a dense turbid solution was formed (see FIG. 1B). As depicted in Table 6, the percent solubilization of the therapeutic agents in a composition prepared using sized lipid vesicle particles was >98%. Advantageously, this high level of solubility was observed even for the therapeutic agent that was added after formation and sizing of the lipid vesicle particles (i.e. the SurA3.K peptide). In contrast, the percent solubilization achieved with non-sized lipid vesicle particles was significantly reduced (16-35%).

As discussed herein, in the pharmaceutical context, reproducibly obtaining a clear solution with a consistently high percentage of solubilized therapeutic agent is an advantageous property. Pharmaceutical products must meet threshold requirements for regulatory approval, including homogeneity and reproducibility. The formation of precipitates and/or a lack of clarity of the solution are not desired properties as they may be indicative of a product in which the components (e.g. therapeutic agents) are not fully soluble. For a cloudy solution, additional processing steps may be required to establish homogeneity, and even then the composition may not be acceptable for pharmaceutical purposes. A slightly hazy solution may be acceptable if it is the salts causing the haze, not precipitated therapeutic agent. However, a clear solution is advantageous.

By using sized lipid vesicle particles, the disclosed methods form a clear solution upon solubilization in a hydrophobic carrier, whereas the non-sized lipid vesicle particle preparations do not. As shown in Example 7, the disclosed methods are reproducible in obtaining clear product (Table 12). Further, as depicted in Table 12, the level of solubilization of the lipids and therapeutic agents is consistently high. After preparing dried lipid/therapeutic agent preparations with 1 mg of each therapeutic agent, the resultant compositions had a percent relative standard deviation (% RSD) ranging from 1.6-2.6% across all five therapeutic agents. With respect to the lipids, after preparing dried lipid/therapeutic agent preparations with 120 mg of DOPC and 12 mg of cholesterol, the resultant compositions had a % RSD of 1.9% for DOPC and 2.0% for cholesterol. Thus, the % RSD for all therapeutic agents and lipids was very low, demonstrating reproducibility.

As used herein, a “hydrophobic carrier” refers to a liquid hydrophobic substance. The term “hydrophobic carrier” may be referred to herein interchangeably as an “oil-based carrier”.

The hydrophobic carrier may be an essentially pure hydrophobic substance or a mixture of hydrophobic substances. Hydrophobic substances that are useful in the methods and compositions described herein are those that are pharmaceutically and/or immunologically acceptable. The carrier is typically a liquid at room temperature (e.g. about 18-25° C.), but certain hydrophobic substances that are not liquids at room temperature may be liquefied, for example by warming, and may also be useful.

Oil or a mixture of oils is a particularly suitable carrier for use in the methods and compositions disclosed herein. Oils should be pharmaceutically and/or immunologically acceptable. Suitable oils include, for example, mineral oils (especially light or low viscosity mineral oil such as Drakeol® 6VR), vegetable oils (e.g., soybean oil such as MS80), nut oils (e.g., peanut oil), or mixtures thereof. Thus, in an embodiment the hydrophobic carrier is a hydrophobic substance such as vegetable oil, nut oil or mineral oil. Animal fats and artificial hydrophobic polymeric materials, particularly those that are liquid at atmospheric temperature or that can be liquefied relatively easily, may also be used.

In some embodiments, the hydrophobic carrier may be, or comprise, Incomplete Freund's Adjuvant (IFA), a mineral oil-based model hydrophobic carrier. In another embodiment, the hydrophobic carrier may be, or comprise, a mannide oleate in mineral oil solution, such as that commercially available as Montanide® ISA 51 (SEPPIC, France). While these carriers are commonly used to prepare water-in-oil emulsions, the present disclosure relates to water-free compositions. As such, these carriers are not emulsified with water in the methods and compositions disclosed herein.

In an embodiment, the hydrophobic carrier is mineral oil or a mannide oleate in mineral oil solution.

In an embodiment, the hydrophobic carrier is Montanide® ISA 51.

In an embodiment, the present disclosure relates to a pharmaceutical composition prepared by the methods disclosed herein.

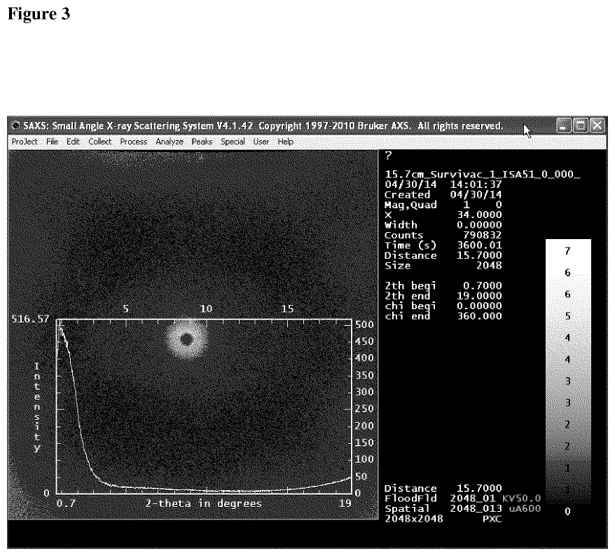

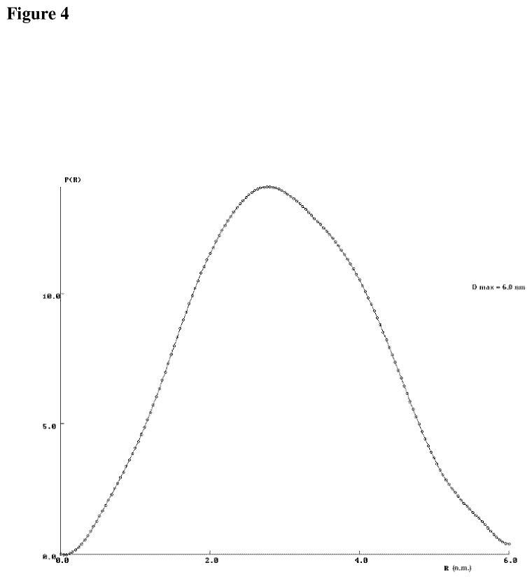

Small angle X-ray scattering (SAXS) can be used for the determination of the nanoscale structure of particle systems in terms of such parameters as averaged particle sizes, shapes, distribution and surface-to-volume ratio. Using the disclosed methods of preparing the dried lipid/therapeutic agent preparation with sized lipid vesicle particles, it has been found that in the hydrophobic carrier the lipids rearrange to form lipid-based structures having a single layer lipid assembly. This is shown in the SAXS pattern and pair-distance distribution function (gaussian curve) of FIGS. 3 and 4.

By “single layer lipid assembly”, it is meant that the lipids form aggregate structures in which the hydrophobic part of the lipids is oriented outwards toward the hydrophobic carrier and the hydrophilic part of the lipids aggregate as a core in the middle. From the SAXS patterns it is not possible to determine if the hydrophilic parts form a continuous single layer membrane (e.g. reverse micelle) or whether the core is a discontinuous aggregate. Irrespective of the configuration, the lipid-based structures comprise a single layer of lipids as opposed to a bilayer that would be found, for example, in liposomes. It is believed that in this configuration, hydrophilic therapeutic agents are in the core of the single layer lipid assembly and the hydrophobic therapeutic agents are solubilized in the non-polar oil.

Without being bound to theory, it is believed based on the examples herein that sizing of the lipid vesicle particles provides the dried lipid/therapeutic agent preparation with favourable properties that allows for better compatibility of the dried lipid/therapeutic agent preparation with the hydrophobic carrier. For example, the sized lipid vesicle particles may allow for an easier rearrangement of the lipid vesicle particles into the lipid-based structures upon solubilization in a hydrophobic carrier, thereby providing a clear product. This is perhaps due to the small, uniform size of the sized lipid vesicle particles. It is also believed that this property allows for the therapeutic agents to be added outside the sized lipid vesicle particles and still be stably formulated in the composition despite various processing steps (e.g. aqueous phase, drying and hydrophobic phase).

Pharmaceutical Compositions

In an embodiment, the present disclosure relates to a stable, water-free pharmaceutical composition comprising one or more lipid-based structures having a single layer lipid assembly, at least one two therapeutic agents, and a hydrophobic carrier. Each of these components is individually described elsewhere herein in greater detail.

As used herein, the terms “pharmaceutical composition”, “composition”, “vaccine composition” or “vaccine” may be used interchangeably, as the context requires.

A pharmaceutical composition as disclosed herein may be administered to a subject in a therapeutically effect amount. As used herein, a “therapeutically effective amount” means an amount of the composition or therapeutic agent effective to provide a therapeutic, prophylactic or diagnostic benefit to a subject, and/or to stimulate, induce, maintain, boost or enhance an immune response in a subject. In some embodiments, a therapeutically effective amount of the composition is an amount capable of inducing a clinical response in a subject in the treatment of a particular disease or disorder. Determination of a therapeutically effective amount of the composition is well within the capability of those skilled in the art, especially in light of the disclosure provided herein. The therapeutically effective amount may vary according to a variety of factors such as the subject's condition, weight, sex and age.

The pharmaceutical compositions disclosed herein are water-free. As used herein, “water-free” means completely or substantially free of water, i.e. the pharmaceutical compositions are not emulsions.

By “completely free of water” it is meant that the compositions contain no water at all. In contrast, the term “substantially free of water” is intended to encompass embodiments where the hydrophobic carrier may still contain small quantities of water, provided that the water is present in the non-continuous phase of the carrier. For example, individual components of the composition may have small quantities of bound water that may not be completely removed by processes such as lyophilization or evaporation and certain hydrophobic carriers may contain small amounts of water dissolved therein. Generally, compositions as disclosed herein that are “substantially free of water” contain, for example, less than about 5%, 4%, 3%, 2%, 1%, 0.5%, 0.1%, 0.05% or 0.01% water on a weight/weight basis of the total weight of the carrier component of the composition. The compositions that still contain small quantities of water do not contain a sufficient amount of water such that an emulsion would be formed.

The pharmaceutical compositions disclosed herein are stable. By “stable”, it is meant that the lipids, therapeutic agents and any other components (e.g. adjuvant and/or T-helper epitope) remain in solubilized form in the hydrophobic carrier. This is an advantageous property of the disclosed compositions. For example, as shown herein, it is possible to formulate different therapeutic agents at different times (e.g. before and after formation and sizing of the lipid vesicle particles) and still obtain a composition that is stable for sufficient periods of time for administration to a subject (Example 4). Moreover, as shown herein, the formulation is stable in a syringe (Example 5).

In an embodiment, the stability of the compositions may be based on the ability to prepare formulations that are a clear or slightly hazy solution. In an embodiment, the stability of the compositions may be based on the ability to prepare formulations that are a clear solution. By “clear solution”, it is meant that the solution does not have a cloudy or hazy appearance. In an embodiment, this may be determined visually by the naked eye by observing a clear solution or by measurement using a spectrophotometer. In an embodiment, the compositions may be visually inspected according to the European Pharmacopoeia (Ph. Eur.), 9^{th} edition, Section 2.9.20.

In an embodiment, the stability of the compositions may be based on the ability to prepare formulations that have no visible precipitates. By “visible precipitate”, it is meant to refer to precipitates that are either located on the wall of the container holding the composition or in the solution of the composition. In an embodiment, this may be determined visually by the naked eye by observing the absence of precipitates or by measurement using a spectrophotometer. In an embodiment, the compositions may be visually inspected according to the European Pharmacopoeia (Ph. Eur.), 9^{th} edition, Section 2.9.20.

In an embodiment, the stability of the compositions may be based on the observed stability of the lipids, therapeutic agents or other components (e.g. adjuvant and/or T-helper epitope) in the dried lipid/therapeutic agent preparation. For example, the stability of the compositions may be based on a substantially consistent therapeutic agent concentration in the dried lipid/therapeutic agent preparation over periods of storage for 1 week, 2 weeks, 3 weeks, 4 weeks, 5 weeks, 6 weeks, 7 weeks, 8 weeks, 9 weeks, 10 weeks, 11 weeks, 12 weeks, or longer. In an embodiment, the stability of the compositions may be based on a substantially consistent therapeutic agent concentration over periods of storage for 1 month, 2 months, 3 months, 4 months, 5 months, 6 months, 7 months, 8 months, 9 months, 10 months, 11 months, 12 months, 13 months, 14 months, 15 months, 16 months, 17 months, 18 months or longer. The stability may, for example and without limitation, be measured by storing the dried lipid/therapeutic agent preparation at −20° C. and/or 5° C. and at various time points removing samples from storage, solubilizing in a hydrophobic carrier and measuring the content of the components. In an embodiment, the lipid and therapeutic agent concentration may be determined by reversed-phase high-performance liquid chromatography (RP-HPLC) analysis as described herein. In an embodiment, the concentration of polynucleotides may be measured by ion-exchange HPLC (IEX-HPLC) analysis as described herein. Stability of the lipids, therapeutic agents and other components in the dried preparation is indicative their ability to be stably solubilized in the hydrophobic carrier.

In an embodiment, the stability of the compositions may further be evaluated by taking into consideration one or more of the following: appearance of the dried preparation (lyophilisate); solubilization time in a hydrophobic carrier; identification and quantification of impurities and/or degradants (e.g. by RP-HPLC); particle size of the lipid-based structures having a single layer lipid assembly (e.g. by SAXS); optical density; viscosity (e.g. as per Ph.Eur. 2.2.9); pH; extractable volume, such as from a syringe (e.g. as per Ph.Eur. 2.9.17), and immunogenicity assays (e.g. ELISpot).

As shown in Example 6 (Tables 10 and 11), the dried lipid/therapeutic agent preparation prepared in accordance with the disclosed methods using sized lipid vesicle particles having a mean particle size of ≤120 nm and PDI of ≤0.1 exhibit long term stability, including in respect of therapeutic agent added after formation and sizing of the lipid vesicle particles. For example, the following observed properties after 0 to 18 months storage at −20° C., which are all well within the acceptance criteria, are indicative of a stable product:

| Characteristic | Observation over 18 months |
|---|---|
| Appearance of dried preparation | Dry, white, non-collapsed cake |
| Appearance of solubilized product | Clear solution, free of particles |
| pH value | 7.1 to 7.3 |
| Therapeutic agent content in | 0.89 to 1.09 mg |
| respect of original 1 mg | |
| T-helper epitope content in | 0.44 to 0.48 mg |
| respect of original 0.5 mg | |
| Polynucleotide adjuvant content | 0.42 to 0.43 mg |
| in respect of original 0.4 mg | |
| Peptide Impurities | None detected |
| DOPC content in respect of | 102.63 to 128.00 mg |
| original 120 mg | |
| Cholesterol content in respect | 11.03 to 12.85 mg |
| of original 12 mg | |
| Lipid degradants | Not detected or minimal (0.2-0.6 mg) |

Likewise, the following observed properties after 0 to 18 months storage at 5° C., which are all well within the acceptance criteria, are indicative of a stable product:

| Characteristic | Observation over 18 months |
|---|---|
| Appearance of dried preparation | Dry, white, non-collapsed cake |
| Appearance of solubilized product | Clear solution, free of particles |
| pH value | 7.2 to 7.5 |
| Therapeutic agent content in | 0.88 to 1.09 mg |
| respect of original 1 mg | |
| T-helper epitope content in | 0.46 to 0.49 mg |
| respect of original 0.5 mg | |
| Polynucleotide adjuvant content | 0.41 to 0.44 mg |
| in respect of original 0.4 mg | |
| Peptide Impurities | None detected |
| DOPC content in respect of | 112.91 to 130.11 mg |
| original 120 mg | |
| Cholesterol content in | 11.03 to 12.26 mg |
| respect of original 12 mg | |
| Lipid degradants | Not detected or minimal (0.2-0.7 mg) |

With respect to stability after solubilization in the hydrophobic carrier, as shown in Example 4 herein, compositions as disclosed herein remain clear and free of particulates for at least 24 hours (Table 8). Moreover, the percent recovery of the lipids, therapeutic agents, adjuvant and T-helper epitope were all within the acceptance criteria, i.e. 85-115% of the average content at t=0 (Table 8). Peptide and lipid impurities were also found to be minimal and well within the acceptance criteria (Table 8).

The compositions disclosed herein were also found to exhibit stability and compatibility within a syringe, as shown in Example 5. Adsorption to the syringe was not observed and there was no significant change in the optical density, viscosity or extractable volume over a 60 minute period (Table 9). Moreover, the compositions remained clear and free of particulates over the 60 minutes in the syringe and percent recovery of the lipids, therapeutic agents, adjuvant and T-helper epitope were all within the acceptance criteria, i.e. 85-115% of the average content at t=0 (Table 9).

In an embodiment, the compositions disclosed herein are stable for a period of at least 30 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 18 hours, at least 24 hours, at least 36 hours, at least 48 hours or longer, after solubilization in the hydrophobic carrier.

In an embodiment, the compositions disclosed herein are stable in a syringe for a period of at least 5 minutes, 10 minutes, 15 minutes, 20 minutes, 25 minutes, 30 minutes, 35 minutes, 40 minutes, 45 minutes, 50 minutes, 55 minutes, 60 minutes, or longer, after solubilization in the hydrophobic carrier and delivery to a syringe. In an embodiment, the syringe has a polycarbonate barrel. In an embodiment, the syringe is a Medallion® syringe.

As described above, the pharmaceutical compositions disclosed herein comprise one or more lipid-based structures having a single layer lipid assembly. As used herein, the term “lipid-based structure” refers to any structure formed by lipids. The lipids that form the lipid-based structures having a single layer lipid assembly are the same lipids as described herein that form the sized lipid vesicle particles.

There are various lipid-based structures which may form, and the compositions disclosed herein may comprise a single type of lipid-based structure having a single layer lipid assembly or comprise a mixture of different lipid-based structures.

In an embodiment, the lipid-based structure having a single layer lipid assembly partially or completely surrounds the therapeutic agent. As an example, the lipid-based structure may be a closed vesicular structure surrounding the therapeutic agent. In an embodiment, the hydrophobic part of the lipids in the vesicular structure is oriented outwards toward the hydrophobic carrier.

As another example, the one or more lipid-based structures having a single layer lipid assembly may comprise aggregates of lipids with the hydrophobic part of the lipids oriented outwards toward the hydrophobic carrier and the hydrophilic part of the lipids aggregating as a core. These structures do not necessarily form a continuous lipid layer membrane. In an embodiment, they are an aggregate of monomeric lipids.

In an embodiment, the one or more lipid-based structures having a single layer lipid assembly comprise reverse micelles. A typical micelle in aqueous solution forms an aggregate with the hydrophilic parts in contact with the surrounding aqueous solution, sequestering the hydrophobic parts in the micelle center. In contrast, in a hydrophobic carrier, an inverse/reverse micelle forms with the hydrophobic parts in contact with the surrounding hydrophobic solution, sequestering the hydrophilic parts in the micelle center. A spherical reverse micelle can package a therapeutic agent with hydrophilic affinity within its core (i.e. internal environment).

Without limitation, the size of the lipid-based structures having a single layer lipid assembly is in the range of from 2 nm (20 A) to 20 nm (200 A) in diameter. In an embodiment, the size of the lipid-based structures having a single layer lipid assembly is between about 2 nm to about 10 nm in diameter. In an embodiment, the size of the lipid-based structures having a single layer lipid assembly is about 2 nm, 3 nm, 4 nm, 5 nm, 6 nm, about 7 nm, about 8 nm, about 9 nm, or about 10 nm in diameter. In an embodiment, the size of the lipid-based structures having a single layer lipid assembly is between about 5 nm to about 10 nm. In an embodiment, the maximum diameter of the lipid-based structures is about 6 nm. In an embodiment, the lipid-based structures of these sizes are reverse micelles.

In an embodiment, one or more of the therapeutic agents are inside the lipid-based structures after solubilization in the hydrophobic carrier. By “inside the lipid-based structure”, it is meant that the therapeutic agent is substantially surrounded by the lipids such that the hydrophilic components of the therapeutic agent are not exposed to the hydrophobic carrier. In an embodiment, the therapeutic agent inside the lipid-based structure is predominantly hydrophilic.

In an embodiment, one or more of the therapeutic agents are outside the lipid-based structures after solubilization in the hydrophobic carrier. By “outside the lipid-based structure”, it is meant that the therapeutic agent is not sequestered within the environment internal to the single layer lipid assembly. In an embodiment, the therapeutic agent outside the lipid-based structure is predominantly hydrophobic.

The pharmaceutical compositions disclosed herein comprise at least two therapeutic agents. Exemplary therapeutic agents are described elsewhere herein, without limitation.

In an embodiment, the compositions comprise 2, 3, 4, 5, 6, 7, 8, 9, 10 or more different therapeutic agents. In an embodiment, the compositions comprise 5-10 different therapeutic agents. In a particular embodiment, the compositions comprise five different therapeutic agents.

In an embodiment, each of the therapeutic agents is independently selected from the group consisting of a peptide antigen, a DNA or RNA polynucleotide that encodes a polypeptide (e.g. mRNA), a hormone, a cytokine, an allergen, a catalytic DNA (deoxyribozyme), a catalytic RNA (ribozyme), an antisense RNA, an interfering RNA (e.g. siRNA or miRNA), an antagomir, a small molecule drug, a biologic drug, an antibody, or a fragment or derivative of any one thereof; or a mixture thereof.

In a particular embodiment, one or more of the therapeutic agents is a peptide antigen. In a particular embodiment, all of the therapeutic agents are peptide antigens. As used herein, the term “peptide antigen” is an antigen that is a protein or a polypeptide. Exemplary embodiments of peptide antigens that may be used in the compositions are described herein, without limitation.

In an embodiment, the composition comprises a single peptide antigen. In an embodiment, the composition comprises 1, 2, 3, 4, 5, 6, 7, 8, 9, 10 or more different peptide antigens. In an embodiment, the composition comprises 5 to 10 different peptide antigens. In a particular embodiment, the composition comprises five different peptide antigens.

By “different” peptide antigens, it is meant that none of the peptide antigens in the pharmaceutical composition have an identical amino acid sequence. The antigens may be derived from the same source (e.g. a virus, bacterium, protozoan, cancer cell, etc.) or from the same protein, but they do not share the same sequence.

In an embodiment, the peptide antigen may be 5 to 120 amino acids in length, 5 to 100 amino acids in length, 5 to 75 amino acids in length, 5 to 50 amino acids in length, 5 to 40 amino acids in length, 5 to 30 amino acids in length, 5 to 20 amino acids in length or 5 to 10 amino acids in length. In an embodiment, the peptide antigen may be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids in length. In an embodiment, the peptide antigen is 8 to 40 amino acids in length. In an embodiment, the peptide antigen is 9 or 10 amino acids in length.

In an embodiment, the one or more peptide antigens are derived from human papillomavirus (HPV), human immunodeficiency virus (HIV), respiratory syncytial virus (RSV), Bacillus anthracis, Plasmodium and/or a survivin polypeptide.

In an embodiment, the one or more of the peptide antigens are derived from RSV, such as for example NKLCEYNVFHNKTFELPRARVNT (SEQ ID NO: 7) and/or NKLSEHKTFCNKTLEQGQMYQINT (SEQ ID NO: 8).

In an embodiment, the one or more of the peptide antigens in the composition are cancer-associated peptide antigens. In an embodiment, all of the peptide antigens in the composition are cancer-associated peptide antigens. Exemplary embodiments of cancer-associated peptide antigens that may be used in the compositions disclosed herein are described below, without limitation. In an embodiment, the cancer-associated peptide antigens may be one or more survivin antigens, such as for example and without limitation, those described herein.

In an embodiment, the one or more peptide antigens are FTELTLGEF (SEQ ID NO: 1), LMLGEFLKL (SEQ ID NO: 2), RISTFKNWPK (SEQ ID NO: 6), STFKNWPFL (SEQ ID NO: 3) or LPPAWQPFL (SEQ ID NO: 4); or any combination thereof. In an embodiment, the composition comprises all five of these peptide antigens (SEQ ID NOs: 1, 2, 3, 4 and 6).

In an embodiment, the one or more of the peptide antigens in the composition are neoantigens. In an embodiment, all of the peptide antigens in the composition are neoantigens. Exemplary embodiments of neoantigens that may be used in the compositions disclosed herein are described below, without limitation.

In an embodiment of the compositions disclosed herein, each of the peptide antigens is, independently, at a concentration of between about 0.05 μg/μl and about 10 μg/μl, 0.1 μg/μl and about 5.0 μg/μl, or about 0.5 μg/μl and about 1.0 μg/μl. In an embodiment of the compositions disclosed herein, each of the peptide antigens is, independently, at a concentration of about 0.1 μg/μl, 0.25 μg/μl, about 0.5 μg/μl, about 0.75 μg/μl, about 1.0 μg/μl, about 1.25 μg/μl, about 1.5 μg/μl, about 1.75 μg/μl, about 2.0 μg/μl, about 2.25 μg/μl or about 2.5 μg/μl. By “independently” it is meant that the amount of each peptide antigen in the composition is independent of the amount of any other and, therefore, each respective peptide antigen may have the same or different concentration as any other peptide antigen. In an embodiment, each of the peptide antigens in the composition is at a concentration of at least about 0.5 μg/μl, more particularly about 1.0 μg/μl.

In an embodiment, the pharmaceutical composition comprises 5 or more different peptide antigens and each peptide antigen is at a concentration of at least about 1.0 μg/μl.

The pharmaceutical compositions disclosed herein comprise a hydrophobic carrier. As used herein, a “hydrophobic carrier” refers to a liquid hydrophobic substance. The term “hydrophobic carrier” may be referred to herein interchangeably as an “oil-based carrier”.

The hydrophobic carrier may be an essentially pure hydrophobic substance or a mixture of hydrophobic substances. Hydrophobic substances that are useful in the methods and compositions described herein are those that are pharmaceutically and/or immunologically acceptable. The carrier is typically a liquid at room temperature (e.g. about 18-25° C.), but certain hydrophobic substances that are not liquids at room temperature may be liquefied, for example by warming, and may also be useful.

Oil or a mixture of oils is a particularly suitable carrier for use in the methods and compositions disclosed herein. Oils should be pharmaceutically and/or immunologically acceptable. Suitable oils include, for example, mineral oils (especially light or low viscosity mineral oil such as Drakeol® 6VR), vegetable oils (e.g., soybean oil such as MS80), nut oils (e.g., peanut oil), or mixtures thereof. Thus, in an embodiment the hydrophobic carrier is a hydrophobic substance such as vegetable oil, nut oil or mineral oil. Animal fats and artificial hydrophobic polymeric materials, particularly those that are liquid at atmospheric temperature or that can be liquefied relatively easily, may also be used.

In some embodiments, the hydrophobic carrier may be, or comprise, Incomplete Freund's Adjuvant (IFA), a mineral oil-based model hydrophobic carrier. In another embodiment, the hydrophobic carrier may be, or comprise, a mannide oleate in mineral oil solution, such as that commercially available as Montanide® ISA 51 (SEPPIC, France). While these carriers are commonly used to prepare water-in-oil emulsions, the present disclosure relates to water-free compositions. As such, these carriers are not emulsified with water in the methods and compositions disclosed herein.

In an embodiment, the hydrophobic carrier is mineral oil or a mannide oleate in mineral oil solution.

In an embodiment, the hydrophobic carrier is Montanide® ISA 51.

The compositions disclosed herein may further comprise one or more additional components as are known in the art (see e.g. Remington's Pharmaceutical Sciences, Mack Publishing Company, Easton, Pa., USA 1985; and The United States Pharmacopoeia: The National Formulary (USP 24 NF19) published in 1999).

In an embodiment, the compositions may additionally comprise an adjuvant, a T-helper epitope, a surfactant and/or an excipient. Exemplary and non-limiting embodiments of adjuvants, T-helper epitopes and surfactants that may be used are described below. In an embodiment, the composition comprises a T-helper epitope and/or adjuvant if the therapeutic agent is one or more peptide antigens.

In an embodiment, the pharmaceutical composition is a clear solution. In an embodiment, the pharmaceutical composition has no visible precipitate.

Immune Responses and Treatment Indications

The compositions disclosed herein may find application in any instance in which it is desired to administer therapeutic agents to a subject. The subject may be a vertebrate, such as a fish, bird or mammal. In an embodiment, the subject is a mammal. In an embodiment, the subject is a human.

In an embodiment, the compositions may be used in methods for treating, preventing or diagnosing a disease, disorder or condition to which the therapeutic agent is targeted. In an embodiment, the methods comprise administering to a subject the pharmaceutical composition as described herein.

In an embodiment, the compositions may be used in methods for modulating an immune response in a subject. As used herein, the term “modulating” is intended to refer to both immunostimulation (e.g. inducing or enhancing an immune response) and immunosuppression (e.g. preventing or decreasing an immune response). Typically, the method would involve one or the other of immunostimulation or immunosuppression, but it is possible that the method could be directed to both. As referred to herein, the “immune response” may either be a cell-mediated (CTL) immune response or an antibody (humoral) immune response.

In some embodiments, the compositions disclosed herein may be used for inducing a cell-mediated immune response to the therapeutic agents (e.g. peptide antigens).

As used herein, to “induce” an immune response is to elicit and/or potentiate an immune response. Inducing an immune response encompasses instances where the immune response is initiated, enhanced, elevated, improved or strengthened to the benefit of the host relative to the prior immune response status, for example, before the administration of a composition disclosed herein.

As used herein, the terms “cell-mediated immune response”, “cellular immunity”, “cellular immune response” or “cytotoxic T-lymphocyte (CTL) immune response” (used interchangeably herein) refer to an immune response characterized by the activation of macrophages and natural killer cells, the production of antigen-specific cytotoxic T lymphocytes and/or the release of various cytokines in response to an antigen. Cytotoxic T lymphocytes are a sub-group of T lymphocytes (a type of white blood cell) which are capable of inducing the death of infected somatic or tumor cells; they kill cells that are infected with viruses (or other pathogens), or that are otherwise damaged or dysfunctional.

Most cytotoxic T cells express T cell receptors that can recognise a specific peptide antigen bound to Class I MHC molecules. Typically, cytotoxic T cells also express CD8 (i.e. CD8+ T cells), which is attracted to portions of the Class I MHC molecule. This affinity keeps the cytotoxic T cell and the target cell bound closely together during antigen-specific activation.

Cellular immunity protects the body by, for example, activating antigen-specific cytotoxic T-lymphocytes (e.g. antigen-specific CD8+ T cells) that are able to lyse body cells displaying epitopes of foreign or mutated antigen on their surface, such as cancer cells displaying tumor-specific antigens (e.g. neoantigens); activating macrophages and natural killer cells, enabling them to destroy intracellular pathogens; and stimulating cells to secrete a variety of cytokines that influence the function of other cells involved in adaptive immune responses and innate immune responses.

Cellular immunity is an important component of the adaptive immune response and following recognition of antigen by cells through their interaction with antigen-presenting cells such as dendritic cells, B lymphocytes and to a lesser extent, macrophages, protect the body by various mechanisms such as:

1. activating antigen-specific cytotoxic T-lymphocytes that are able to induce apoptosis in body cells displaying epitopes of foreign or mutated antigen on their surface, such as cancer cells displaying tumor-specific antigens;

2. activating macrophages and natural killer cells, enabling them to destroy intracellular pathogens; and

3. stimulating cells to secrete a variety of cytokines that influence the function of other cells involved in adaptive immune responses and innate immune responses.

Cell-mediated immunity is most effective in removing virus-infected cells, but also participates in defending against fungi, protozoans, cancers, and intracellular bacteria. It also plays a major role in transplant rejection.

Since cell-mediated immunity involves the participation of various cell types and is mediated by different mechanisms, several methods could be used to demonstrate the induction of immunity following vaccination. These could be broadly classified into detection of: i) specific antigen presenting cells; ii) specific effector cells and their functions and iii) release of soluble mediators such as cytokines.

i) Antigen presenting cells: Dendritic cells and B cells (and to a lesser extent macrophages) are equipped with special immunostimulatory receptors that allow for enhanced activation of T cells, and are termed professional antigen presenting cells (APC). These immunostimulatory molecules (also called co-stimulatory molecules) are up-regulated on these cells following infection or vaccination, during the process of antigen presentation to effector cells such as CD4 and CD8 cytotoxic T cells. Such co-stimulatory molecules (such as CD40, CD80, CD86, MHC class I or MHC class II) can be detected, for example, by using flow cytometry with fluorochrome-conjugated antibodies directed against these molecules along with antibodies that specifically identify APC (such as CD11c for dendritic cells).

ii) Cytotoxic T cells: (also known as Tc, killer T cell, or cytotoxic T-lymphocyte (CTL)) are a sub-group of T cells which induce the death of cells that are infected with viruses (and other pathogens), or expressing tumor antigens. These CTLs directly attack other cells carrying certain foreign or abnormal molecules on their surface. The ability of such cellular cytotoxicity can be detected using in vitro cytolytic assays (chromium release assay). Thus, induction of adaptive cellular immunity can be demonstrated by the presence of such cytotoxic T cells, wherein, when antigen loaded target cells are lysed by specific CTLs that are generated in vivo following vaccination or infection.

Naive cytotoxic T cells are activated when their T cell receptor (TCR) strongly interacts with a peptide-bound MHC class I molecule. This affinity depends on the type and orientation of the antigen/MHC complex, and is what keeps the CTL and infected cell bound together. Once activated the CTL undergoes a process called clonal expansion in which it gains functionality, and divides rapidly, to produce an army of “armed”-effector cells. Activated CTL will then travel throughout the body in search of cells bearing that unique MHC Class I+peptide. This could be used to identify such CTLs in vitro by using peptide-MHC Class I tetramers in flow cytometric assays.

When exposed to these infected or dysfunctional somatic cells, effector CTL release perforin and granulysin: cytotoxins which form pores in the target cell's plasma membrane, allowing ions and water to flow into the infected cell, and causing it to burst or lyse. CTL release granzyme, a serine protease that enters cells via pores to induce apoptosis (cell death). Release of these molecules from CTL can be used as a measure of successful induction of cell-mediated immune response following vaccination. This can be done by enzyme linked immunosorbant assay (ELISA) or enzyme linked immunospot assay (ELISPOT) where CTLs can be quantitatively measured. Since CTLs are also capable of producing important cytokines such as IFN-γ, quantitative measurement of IFN-γ-producing CD8 cells can be achieved by ELISPOT and by flowcytometric measurement of intracellular IFN-γ in these cells.

CD4+“helper” T cells: CD4+ lymphocytes, or helper T cells, are immune response mediators, and play an important role in establishing and maximizing the capabilities of the adaptive immune response. These cells have no cytotoxic or phagocytic activity; and cannot kill infected cells or clear pathogens, but, in essence “manage” the immune response, by directing other cells to perform these tasks. Two types of effector CD4+ T helper cell responses can be induced by a professional APC, designated Th1 and Th2, each designed to eliminate different types of pathogens.

Helper T cells express T cell receptors (TCR) that recognize antigen bound to Class II MHC molecules. The activation of a naive helper T cell causes it to release cytokines, which influences the activity of many cell types, including the APC that activated it. Helper T cells require a much milder activation stimulus than cytotoxic T cells. Helper T cells can provide extra signals that “help” activate cytotoxic cells. Two types of effector CD4+ T helper cell responses can be induced by a professional APC, designated Th1 and Th2, each designed to eliminate different types of pathogens. The two Th cell populations differ in the pattern of the effector proteins (cytokines) produced. In general, Th1 cells assist the cell-mediated immune response by activation of macrophages and cytotoxic T cells; whereas Th2 cells promote the humoral immune response by stimulation of B cells for conversion into plasma cells and by formation of antibodies. For example, a response regulated by Th1 cells may induce lgG2a and lgG2b in mouse (IgGI and lgG3 in humans) and favor a cell mediated immune response to an antigen. If the IgG response to an antigen is regulated by Th2 type cells, it may predominantly enhance the production of IgGI in mouse (lgG2 in humans). The measure of cytokines associated with Th1 or Th2 responses will give a measure of successful vaccination. This can be achieved by specific ELISA designed for Th1-cytokines such as IFN-γ, IL-2, IL-12, TNF-α and others, or Th2-cytokines such as IL-4, IL-5, IL10 among others.

iii) Measurement of cytokines: released from regional lymph nodes gives a good indication of successful immunization. As a result of antigen presentation and maturation of APC and immune effector cells such as CD4 and CD8 T cells, several cytokines are released by lymph node cells. By culturing these LNC in vitro in the presence of antigen, an antigen-specific immune response can be detected by measuring release if certain important cytokines such as IFN-γ, IL-2, IL-12, TNF-α and GM-CSF. This could be done by ELISA using culture supernatants and recombinant cytokines as standards.

Successful immunization may be determined in a number of ways known to the skilled person including, but not limited to, hemagglutination inhibition (HAJ) and serum neutralization inhibition assays to detect functional antibodies; challenge studies, in which vaccinated subjects are challenged with the associated pathogen to determine the efficacy of the vaccination; and the use of fluorescence activated cell sorting (FACS) to determine the population of cells that express a specific cell surface marker, e.g. in the identification of activated or memory lymphocytes. A skilled person may also determine if immunization with a composition as disclosed herein elicited an antibody and/or cell mediated immune response using other known methods. See, for example, Coligan et al., ed. Current Protocols in Immunology, Wiley Interscience, 2007.

In an embodiment, the composition disclosed herein is capable of generating an enhanced cell-mediated immune response against one or more of the therapeutic agents (e.g. peptide antigens) in the composition that is at least 2-fold, at least 3-fold, at least 4-fold, at least 5-fold, at least 6-fold, at least 7-fold, at least 8-fold, at least 9-fold or at least 10-fold greater than when the antigens are formulated in an aqueous-based vaccine formulation. By “aqueous-based vaccine”, it is meant a vaccine that comprises identical components as the compositions disclosed herein, with the exception that the hydrophobic carrier is replaced with an aqueous carrier and the aqueous-based vaccine does not comprise lipid-based structures.

In an embodiment, the composition disclosed herein is capable of generating the enhanced cell-mediated immune response with only a single administration of the composition. Thus, in an embodiment, the compositions disclosed herein are for delivery of the therapeutic agents (e.g. peptide antigens) by single administration.

In an embodiment, the compositions disclosed herein may be used for inducing an antibody immune response to the therapeutic agents (e.g. peptide antigens).

An “antibody immune response” or “humoral immune response” (used interchangeably herein), as opposed to cell-mediated immunity, is mediated by secreted antibodies which are produced in the cells of the B lymphocyte lineage (B cells). Such secreted antibodies bind to antigens, such as for example those on the surfaces of foreign substances, pathogens (e.g. viruses, bacteria, etc.) and/or cancer cells, and flag them for destruction.

As used herein, “humoral immune response” refers to antibody production and may also include, in addition or alternatively, the accessory processes that accompany it, such as for example the generation and/or activation of T-helper 2 (Th2) or T-helper 17 (Th17) cells, cytokine production, isotype switching, affinity maturation and memory cell activation. “Humoral immune response” may also include the effector functions of an antibody, such as for example toxin neutralization, classical complement activation, and promotion of phagocytosis and pathogen elimination. The humoral immune response is often aided by CD4+Th2 cells and therefore the activation or generation of this cell type may also be indicative of a humoral immune response.

An “antibody” is a protein comprising one or more polypeptides substantially or partially encoded by immunoglobulin genes or fragments of immunoglobulin genes. The recognized immunoglobulin genes include the κ, λ, α, γ, δ, ε and μ constant region genes, as well as myriad immunoglobulin variable region genes. Light chains are classified as either κ or λ. Heavy chains are classified as γ, μ, α, δ, or ε, which in turn define the immunoglobulin classes, IgG, IgM, IgA, IgD and IgE, respectively. A typical immunoglobulin (antibody) structural unit comprises a protein containing four polypeptides. Each antibody structural unit is composed of two identical pairs of polypeptide chains, each having one “light” and one “heavy” chain. The N-terminus of each chain defines a variable region primarily responsible for antigen recognition. Antibody structural units (e.g. of the IgA and IgM classes) may also assemble into oligomeric forms with each other and additional polypeptide chains, for example as IgM pentamers in association with the J-chain polypeptide.

Antibodies are the antigen-specific glycoprotein products of a subset of white blood cells called B lymphocytes (B cells). Engagement of antigen with antibody expressed on the surface of B cells can induce an antibody response comprising stimulation of B cells to become activated, to undergo mitosis and to terminally differentiate into plasma cells, which are specialized for synthesis and secretion of antigen-specific antibody.

B cells are the sole producers of antibodies during an immune response and are thus a key element to effective humoral immunity. In addition to producing large amounts of antibodies, B cells also act as antigen-presenting cells and can present antigenic peptide to T cells, such as T helper CD4 or cytotoxic CD8+ T cells, thus propagating the immune response. B cells, as well as T cells, are part of the adaptive immune response. During an active immune response, induced for example by either vaccination or natural infection, antigen-specific B cells are activated and clonally expand. During expansion, B cells evolve to have higher affinity for the epitope. Proliferation of B cells can be induced indirectly by activated T-helper cells, and also directly through stimulation of receptors, such as the TLRs.

Antigen presenting cells, such as dendritic cells and B cells, are drawn to vaccination sites and can interact with antigens and adjuvants contained in a vaccine composition. Typically, the adjuvant stimulates the cells to become activated and the antigen provides the blueprint for the target. Different types of adjuvants may provide different stimulation signals to cells. For example, polyI:C (a TLR3 agonist) can activate dendritic cells, but not B cells. Adjuvants such as Pam3Cys, Pam2Cys and FSL-1 are especially adept at activating and initiating proliferation of B cells, which is expected to facilitate the production of an antibody response (Moyle 2008; So 2012).

A humoral immune response is one of the common mechanisms for effective infectious disease vaccines (e.g. to protect against viral or bacterial invaders). However, a humoral immune response can also be useful for combating cancer. Whereas a cancer vaccine is typically designed to produce a cell-mediated immune response that can recognize and destroy cancer cells, B cell mediated responses may target cancer cells through other mechanisms which may in some instances cooperate with a cytotoxic T cell for maximum benefit. Examples of B cell mediated (e.g. humoral immune response mediated) anti-tumor responses include, without limitation: 1) Antibodies produced by B cells that bind to surface antigens (e.g. neoantigens) found on tumor cells or other cells that influence tumorigenesis. Such antibodies can, for example. induce killing of target cells through antibody-dependant cell-mediated cytotoxicity (ADCC) or complement fixation, potentially resulting in the release of additional antigens that can be recognized by the immune system; 2) Antibodies that bind to receptors on tumor cells to block their stimulation and in effect neutralize their effects; 3) Antibodies that bind to factors released by or associated with a tumor or tumor-associated cells to modulate a signaling or cellular pathway that supports cancer; and 4) Antibodies that bind to intracellular targets and mediate anti-tumor activity through a currently unknown mechanism.

One method of evaluating an antibody response is to measure the titers of antibodies reactive with a particular antigen. This may be performed using a variety of methods known in the art such as enzyme-linked immunosorbent assay (ELISA) of antibody-containing substances obtained from animals. For example, the titers of serum antibodies which bind to a particular antigen may be determined in a subject both before and after exposure to the antigen. A statistically significant increase in the titer of antigen-specific antibodies following exposure to the antigen would indicate the subject had mounted an antibody response to the antigen.

Without limitation, other assays that may be used to detect the presence of an antigen-specific antibody include immunological assays (e.g. radioimmunoassay (RIA)), immunoprecipitation assays, and protein blot (e.g. Western blot) assays; and neutralization assays (e.g., neutralization of viral infectivity in an in vitro or in vivo assay).

The compositions disclosed herein may be useful for treating or preventing diseases and/or disorders ameliorated by a cell-mediated immune response or a humoral immune response. The compositions disclosed herein may find application in any instance in which it is desired to administer therapeutic agents (e.g. peptide antigens) to a subject to induce a cell-mediated immune response or a humoral immune response. In an embodiment, the compositions may find application for the delivery of a personalized vaccine, e.g. comprising neoantigens.

In an embodiment, the present disclosure relates to a method comprising administering the composition as described herein to a subject in need thereof. In an embodiment, the method is for the treatment and/or prevention of a disease, disorder or condition in a subject. In an embodiment, the method is for the treatment and/or prevention of an infectious disease or cancer.

In an embodiment, the method is for inducing an antibody immune response and/or cell-mediated immune response to the therapeutic agents (e.g. peptide antigens) in said subject. In an embodiment, such method is for the treatment and/or prevention of an infectious disease or cancer.

“Treating” or “treatment of”, or “preventing” or “prevention of”, as used herein, refers to an approach for obtaining beneficial or desired results. Beneficial or desired results can include, but are not limited to, alleviation or amelioration of one or more symptoms or conditions, diminishment of extent of disease, stabilisation of the state of disease, prevention of development of disease, prevention of spread of disease, delay or slowing of disease progression (e.g. suppression), delay or slowing of disease onset, conferring protective immunity against a disease-causing agent and amelioration or palliation of the disease state. “Treating” or “preventing” can also mean prolonging survival of a patient beyond that expected in the absence of treatment and can also mean inhibiting the progression of disease temporarily or preventing the occurrence of disease, such as by preventing infection in a subject. “Treating” or “preventing” may also refer to a reduction in the size of a tumor mass, reduction in tumor aggressiveness, etc.

Treating” may be distinguished from “preventing” in that “treating” typically occurs in a subject who already has a disease or disorder, or is known to have already been exposed to an infectious agent, whereas “preventing” typically occurs in a subject who does not have a disease or disorder, or is not known to have been exposed to an infectious agent. As will be appreciated, there may be overlap in treatment and prevention. For example, it is possible to be “treating” a disease in a subject, while at same time “preventing” symptoms or progression of the disease. Moreover, at least in the context of vaccination, “treating” and “preventing” may overlap in that the treatment of a subject is to induce an immune response that may have the subsequent effect of preventing infection by a pathogen or preventing the underlying disease or symptoms caused by infection with the pathogen. These preventive aspects are encompassed herein by expressions such as “treatment of an infectious disease” or “treatment of cancer”.

In an embodiment, the compositions disclosed herein may be used for treating and/or preventing an infectious disease, such as caused by a viral infection, in a subject in need thereof. The subject may be infected with a virus or may be at risk of developing a viral infection. Viral infections that may be treated and/or prevented by the use or administration of a composition as disclosed herein, without limitation, Cowpoxvirus, Vaccinia virus, Pseudocowpox virus, Human herpesvirus 1, Human herpesvirus 2, Cytomegalovirus, Human adenovirus A-F, Polyomavirus, Human papillomavirus (HPV), Parvovirus, Hepatitis A virus, Hepatitis B virus, Hepatitis C virus, Human immunodeficiency virus, Orthoreovirus, Rotavirus, Ebola virus, parainfluenza virus, influenza A virus, influenza B virus, influenza C virus, Measles virus, Mumps virus, Rubella virus, Pneumovirus, respiratory syncytial virus (RSV), Rabies virus, California encephalitis virus, Japanese encephalitis virus, Hantaan virus, Lymphocytic choriomeningitis virus, Coronavirus, Enterovirus, Rhinovirus, Poliovirus, Norovirus, Flavivirus, Dengue virus, West Nile virus, Yellow fever virus and varicella. In a particular embodiment, the viral infection is Human papillomavirus, Ebola virus, respiratory syncytial virus or an influenza virus.

In an embodiment, the compositions disclosed herein may be used for treating and/or preventing an infectious disease, such as caused by a non-viral pathogen (such as a bacterium or protozoan) in a subject in need thereof. The subject may be infected with the pathogen or may be at risk of developing an infection by the pathogen. Without limitation, exemplary bacterial pathogens may include Anthrax (Bacillus anthracis), Brucella, Bordetella pertussis, Candida, Chlamydia pneumoniae, Chlamydia psittaci, Cholera, Clostridium botulinum, Coccidioides immitis, Cryptococcus, Diphtheria, Escherichia coli 0157: H7, Enterohemorrhagic Escherichia coli, Enterotoxigenic Escherichia coli, Haemophilus influenzae, Helicobacter pylori, Legionella, Leptospira, Listeria, Meningococcus, Mycoplasma pneumoniae, Mycobacterium, Pertussis, Pneumonia, Salmonella, Shigella, Staphylococcus, Streptococcus pneumoniae and Yersinia enterocolitica. In a particular embodiment, the bacterial infection is Anthrax. Without limitation, exemplary protozoan pathogens may include those of the genus Plasmodium (Plasmodium falciparum, Plasmodium malariae, Plasmodium vivax, Plasmodium ovale or Plasmodium knowlesi), which cause malaria.

In an embodiment, the compositions disclosed herein may be for use in treating and/or preventing cancer in a subject in need thereof. The subject may have cancer or may be at risk of developing cancer.

As used herein, the terms “cancer”, “cancer cells”, “tumor” and “tumor cells”, (used interchangeably) refer to cells that exhibit abnormal growth, characterized by a significant loss of control of cell proliferation or cells that have been immortalized. The term “cancer” or “tumor” includes metastatic as well as non-metastatic cancer or tumors. A cancer may be diagnosed using criteria generally accepted in the art, including the presence of a malignant tumor.

Without limitation, cancers that may be capable of being treated and/or prevented by the use or administration of a composition as disclosed herein include carcinoma, adenocarcinoma, lymphoma, leukemia, sarcoma, blastoma, myeloma, and germ cell tumors. Without limitation, particularly suitable embodiments may include glioblastoma, multiple myeloma, ovarian cancer, breast cancer, fallopian tube cancer, prostate cancer or peritoneal cancer. In one embodiment, the cancer may be caused by a pathogen, such as a virus. Viruses linked to the development of cancer are known to the skilled person and include, but are not limited to, human papillomaviruses (HPV), John Cunningham virus (JCV), Human herpes virus 8, Epstein Barr Virus (EBV), Merkel cell polyomavirus, Hepatitis C Virus and Human T cell leukaemia virus-1. In an embodiment, the cancer is one that expresses one or more tumor-specific neoantigens.

In a particular embodiment, the cancer is breast cancer, ovarian cancer, prostate cancer, fallopian tube cancer, peritoneal cancer, glioblastoma or diffuse large B cell lymphoma.

The methods and compositions disclosed herein may be useful for either the treatment or prophylaxis of cancer; for example, a reduction of the severity of cancer (e.g. size of the tumor, aggressiveness and/or invasiveness, malignancy, etc.) or the prevention of cancer recurrences.

In an embodiment, the method for treating and/or preventing cancer first comprises identifying one or more neoantigens or neoepitopes in the patients' tumor cells. The skilled person will understand methods known in the art that can be used to identify the one or more neoantigens (see, for example, Srivastava 2015 and the references cited therein). As an exemplary embodiment, whole genome/exome sequencing may be used to identify mutated neoantigens that are uniquely present in a tumor of an individual patient. The collection of identified neoantigens can be analyzed to select (e.g. based on algorithms) a specific, optimized subset of neoantigens and/or neoepitopes for use as a personalized cancer vaccine.

Having identified and selected one or more neoantigens, one of skill in the art will appreciate that there are a variety of ways in which to produce such neoantigens either in vitro or in vivo. The neoantigenic peptides may be produced by any method known the art and then may be formulated into a composition or kit as described herein and administered to a subject.

In an embodiment, upon administration to a subject, the composition induces a tumor-specific immune response in the treatment of cancer. By this it is meant that the immune response specifically targets the tumor cells without a significant effect on normal cells of the body which do not express the neoantigen. Further, in an embodiment, the composition may comprise at least one patient-specific neoepitope such that the tumor-specific immune response is patient-specific for the subject or a subset of subjects, i.e. a personalized immunotherapy.

The composition as disclosed herein may be administered by any suitable route. In an embodiment, the route of administration is subcutaneous injection.

In an embodiment in which the composition is for administration by injection, the pharmaceutical compositions as disclosed herein may be formulated as a microdose. As used herein, by “microdose volume” it is meant a single dose volume of less than 100 μl. In some embodiments, the microdose volume is about 50 μl, about 55 μl, about 60 μl, about 65 μl, about 70 μl, about 75 μl, about 80 μl, about 85 μl, about 90 μl or about 95 μl of the composition. In some embodiments, the microdose volume is between about 50 μl to about 75 μl of the composition. In some embodiments, the microdose volume is about 50 μl or exactly 50 μl. In an embodiment, by practice of the methods disclosed herein and use of the compositions disclosed herein, the microdose volume is capable of being formulated with multiple different peptide antigens at a total peptide antigen concentration of more than 5 μg in the microdose, and the microdose volume is capable of inducing an antibody and/or CTL immune response in a human subject.

Kits

The compositions disclosed herein are optionally provided to a user as a kit. In an embodiment, the kit is for preparing a composition for the treatment, prevention and/or diagnosis of a disease, disorder or condition. In an embodiment, the kit is for preparing a composition for inducing an antibody and/or CTL immune response.

In an embodiment, a kit of the present disclosure comprises a container comprising a dried lipid/therapeutic agent preparation prepared by the methods disclosed herein and a container comprising a hydrophobic carrier.

In another embodiment, a kit of the present disclosure comprises a container comprising a dried lipid/therapeutic agent preparation prepared by the methods disclosed herein. In such embodiment, the kit does not include the hydrophobic carrier, but rather the hydrophobic carrier is supplied separately or is already in possession by the end user.

The dried lipid/therapeutic agent preparation may be any of those described herein. In an embodiment, the dried lipid/therapeutic agent preparation comprises five or more different peptide antigens. In an embodiment, the peptide antigens are derived from the survivin protein. In an embodiment, the dried lipid/therapeutic agent preparation comprises the peptide antigens FTELTLGEF (SEQ ID NO: 1); LMLGEFLKL (SEQ ID NO: 2); STFKNWPFL (SEQ ID NO: 3); LPPAWQPFL (SEQ ID NO: 4); and RISTFKNWPK (SEQ ID NO: 6).

The hydrophobic carrier is as described herein, and in an embodiment is mineral oil or a mannide oleate in mineral oil solution.

The kits can further comprise one or more additional reagents, packaging materials, and an instruction set or user manual detailing preferred methods of using the kit components. In an embodiment, the containers are vials.

Components of the Methods, Dried Preparations, Compositions, Uses & Kits

The methods, dried preparations, compositions, uses and kits disclosed herein are used with or comprise two or more therapeutic agents and may further be used with or comprise, without limitation, one or more additional components, such as for example a T-helper epitope, an adjuvant and a surfactant. While exemplary embodiments of these components are described herein, it will be appreciated that other components may also be used, such as excipients, preservatives, or other inactive ingredients.

As used herein, the term “therapeutic agent” does not include or encompass a T-helper epitope or an adjuvant, which are separately described below and are different components that may or may not be included in the methods, dried preparations, compositions, uses and kits disclosed herein. Further, in an embodiment, a T-helper epitope and/or an adjuvant are included only when the therapeutic agents include an antigen.

Therapeutic Agents

Unless specifically stated otherwise, the term “therapeutic agent” as used in this section describes and encompasses both “first therapeutic agents” and “second therapeutic agents” in respect of the methods disclosed herein. The first and second therapeutic agents may be any one or more of the therapeutic agents as described herein, or any combination thereof. However, if a specific therapeutic agent is used as a first therapeutic agent then an identical therapeutic agent will not be used as a second therapeutic agent. In respect of the dried preparations, compositions and kits disclosed herein, the therapeutic agent may be any one or more of the therapeutic agents as described herein, or any combination thereof

Therapeutic agents that can be used in the methods, dried preparations, compositions, uses and kits disclosed herein include any molecule, substance or compound that is capable of providing a therapeutic activity, response or effect in the treatment or prevention of a disease, disorder or condition, including diagnostic and prophylactic agents. The term “therapeutic agent” includes molecules, compounds and substances, or parts thereof, commonly referred to as “active pharmaceutical ingredients” or “active ingredients”, which represent the component of a medicine that is biologically active.

As used herein, the “therapeutic agent” is not a T-helper epitope or an adjuvant, which are described separately below.

The therapeutic agents include antigens, drugs and other agents including, but not limited to, those listed in the United States Pharmacopeia and in other known pharmacopeias. Therapeutic agents may be used in the practice of the present invention with or without any chemical modification. Therapeutic agents include proteins, polypeptides, peptides, polynucleotides, polysaccharides, and drugs (e.g. small molecules or biologics).

In an embodiment, the therapeutic agent is a peptide antigen, a DNA or RNA polynucleotide that encodes a polypeptide, a hormone, a cytokine, an allergen, a catalytic DNA (deoxyribozyme), a catalytic RNA (ribozyme), an antisense RNA, an interfering RNA, an antagomir, a small molecule drug, a biologic drug, an antibody, or a fragment or derivative of any one thereof; or a mixture thereof.

The methods disclosed herein are for formulating multiple different therapeutic agents in a single composition. In an embodiment, the methods disclosed herein are for formulating 2, 3, 4, 5, 6, 7, 8, 9, 10 or more different therapeutic agents in a single composition. In an embodiment, the methods disclosed herein are for formulating 2 to 10 different therapeutic agents in a single composition. In an embodiment, the methods disclosed herein are for formulating 2, 3, 4, or 5 different therapeutic agents in a single composition. In an embodiment, the methods are for formulating five different therapeutic agents in a single composition.

In an embodiment, all of the therapeutic agents used in the methods, dried preparations, compositions and kits may be of the same type (e.g. all peptide antigens, all small molecule drugs, all polynucleotides encoding polypeptides, etc.). In other embodiments, the therapeutic agents may be of different types (e.g. one or more peptide antigens in combination with one or more small molecule drugs).

In an embodiment, the therapeutic agent is one that is not compatible (e.g. insoluble or unstable) with one or both of an aqueous solution or a hydrophobic solution or both. In an embodiment, the therapeutic agent is hydrophilic or substantially hydrophilic and is not naturally compatible in a hydrophobic environment. In an embodiment, the therapeutic agent is hydrophobic or substantially hydrophobic and is not naturally compatible in a hydrophilic (e.g. aqueous) environment.

In an embodiment, with respect specifically to a second therapeutic agent, the second therapeutic agent is one that is not compatible with size extrusion procedures (e.g. precipitates under high pressure extrusion through a membrane, such for example at 1000-5000 psi with a 0.22 μm membrane, 0.1 μm membrane and/or 0.08 μm membrane).

Exemplary embodiments of therapeutics agents are described below, without limitation.

Peptide Antigens

In an embodiment, the one or more of the therapeutic agents is a peptide antigen. In an embodiment, all of the therapeutic agents are peptide antigens.

As used herein, the term “antigen” refers to any substance or molecule that can bind specifically to components of the immune system. In some embodiments, suitable antigens are those that are capable of inducing or generating an immune response in a subject. An antigen that is capable of inducing an immune response is said to be immunogenic, and may also be called an immunogen. Thus, as used herein, the term “antigen” includes immunogens and the terms may be used interchangeably unless specifically stated otherwise.

As used herein, the term “peptide antigen” is an antigen as defined above that is a protein or a polypeptide. In an embodiment, the peptide antigen may be derived from a microorganism, such as for example a live, attenuated, inactivated or killed bacterium, virus or protozoan, or part thereof. In an embodiment, the peptide antigen may be derived from an animal, such as for example a human, or an antigen that is substantially related thereto.

As used herein, the term “derived from” encompasses, without limitation: a peptide antigen that is isolated or obtained directly from an originating source (e.g. a subject); a synthetic or recombinantly generated peptide antigen that is identical or substantially related to a peptide antigen from an originating source; or a peptide antigen which is made from a peptide antigen of an originating source or a fragment thereof. When it is stated that a peptide antigen is “from” a source, the term “from” may be equated with “derived from”. The term “substantially related”, in this context, means that the peptide antigen may have been modified by chemical, physical or other means (e.g. sequence modification), but that the resultant product remains capable of generating an immune response to the original peptide antigen and/or to the disease or disorder associated with the original antigen. “Substantially related” includes variants and/or derivatives of the native peptide antigen.

In an embodiment, the peptide antigen can be isolated from a natural source. In some embodiments, the peptide antigen may be purified to be from about 90% to about 95% pure, from about 95% to about 98% pure, from about 98% to about 99% pure, or greater than 99% pure.

In an embodiment, the peptide antigen can be recombinantly generated, such as for example by expression in vitro or in vivo.

In an embodiment, the peptide antigen is a synthetically produced polypeptide based on a sequence of amino acids of a native target protein. The peptide antigen can be synthesized, in whole or in part, using chemical methods well known in the art (see e.g., Caruthers 1980, Horn 1980, Banga 1995). For example, peptide synthesis can be performed using various solid-phase techniques (see e.g., Roberge 1995, Merrifield 1997) and automated synthesis may be achieved, e.g., using the ABI 431A Peptide Synthesizer (Perkin Elmer) in accordance with the instructions provided by the manufacturer.

As used interchangeably herein, the terms “variant” or “modified variant” refer to therapeutic agents that have been modified by any chemical, physical or other means to provide an altered therapeutic agent. The modified variant may have one or more improved characteristics as compared to the unmodified counterpart (e.g. solubility, stability, activity, etc.). Depending on the type of therapeutic agent (e.g. peptide antigen, hormone, catalytic DNA or RNA, etc.), different types of modifications may be known in the art and may be applied to prepare a modified variant.

In the context of peptide antigens, many different types of peptide modifications are known in the art and may be used in the practice of the present invention. For example, and without limitation, the peptide antigen may be modified to improve its solubility, stability and/or immunogenicity. Non-limiting examples of modifications that may be made include N-terminal modifications, C-terminal modifications, amidation, acetylation, peptide cyclization by creating disulfide bridges, phosphorylation, methylation, conjugation to other molecules (e.g. BSA, KLH, OVA), PEGylation and the inclusion of unnatural amino acids.

In an embodiment, the modification may be an amino acid sequence modification, e.g. deletion, substitution or insertion. The substitution may be a conservative amino acid substitution or a non-conservative amino acid substitution. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing. Specific, non-limiting examples of a conservative substitution include the following examples:

| Original Residue | Conservative Substitution |
|---|---|
| Ala | Ser |
| Arg | Lys |
| Asn | Gln, His |
| Asp | Glu |
| Cys | Ser |
| Gln | Asn |
| Glu | Asp |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Val | Ile, Leu |

In an embodiment, the peptide antigen may be 5 to 120 amino acids in length, 5 to 100 amino acids in length, 5 to 75 amino acids in length, 5 to 50 amino acids in length, 5 to 40 amino acids in length, 5 to 30 amino acids in length, 5 to 20 amino acids in length or 5 to 10 amino acids in length. In an embodiment, the peptide antigen may be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids in length. In an embodiment, the peptide antigen is 8 to 40 amino acids in length. In an embodiment, the peptide antigen is 9 or 10 amino acids in length.

In an embodiment, the peptide antigen comprises at least one B cell epitope, at least one CTL epitope or any combination thereof.

B cell epitopes are epitopes recognized by B cells and by antibodies. B cell peptide epitopes are typically at least five amino acids, more often at least six amino acids, still more often at least seven or eight amino acids in length, and may be continuous (“linear”) or discontinuous (“conformational”); the latter being formed, for example, by the folding of a protein to bring non-contiguous parts of the primary amino acid sequence into physical proximity.

CTL epitopes are molecules recognized by cytotoxic T lymphocytes. CTL epitopes are typically presented on the surface of an antigen-presenting cell, complexed with MHC molecules. As used herein, the term “CTL epitope” refers to a peptide which is substantially the same as a natural CTL epitope of an antigen. The CTL epitope may be modified as compared to its natural counterpart, such as by one or two amino acids. Unless otherwise stated, reference herein to a CTL epitope is to an unbound molecule that is capable of being taken up by cells and presented on the surface of an antigen-presenting cell.

The CTL epitope should typically be one that is amendable to recognition by T cell receptors so that a cell-mediated immune response can occur. For peptides, CTL epitopes may interact with class I or class II MHC molecules. CTL epitopes presented by MHC class I molecules are typically peptides between 8 and 15 amino acids in length, and more often between 9 and 11 amino acids in length. CTL epitopes presented by MHC class II molecules are typically peptides between 5 and 24 amino acids in length, and more often between 13 and 17 amino acids in length. If the antigen is larger than these sizes, it will be processed by the immune system into fragments of a size more suitable for interaction with MHC class I or II molecules. Therefore, CTL epitopes may be part of larger peptide antigen than those mentioned above.

Many CTL epitopes are known. Several techniques of identifying additional CTL epitopes are recognized in the art. In general, these involve preparing a molecule which potentially provides a CTL epitope and characterizing the immune response to that molecule.

In an embodiment, the peptide antigen may be one that is associated with cancer, an infectious disease, an addiction disease, or any other disease or disorder.

Viruses, or parts thereof, from which a peptide antigen may be derived include for example, and without limitation, Cowpoxvirus, Vaccinia virus, Pseudocowpox virus, herpes virus, Human herpesvirus 1, Human herpesvirus 2, Cytomegalovirus, Human adenovirus A-F, Polyomavirus, human papillomavirus (HPV), Parvovirus, Hepatitis A virus, Hepatitis B virus, Hepatitis C virus, human immunodeficiency virus (HIV), Seneca Valley virus (SVV), Orthoreovirus, Rotavirus, Ebola virus, parainfluenza virus, influenza virus (e.g. H5N1 influenza virus, influenza A virus, influenza B virus, influenza C virus), Measles virus, Mumps virus, Rubella virus, Pneumovirus, respiratory syncytial virus, respiratory syncytial virus (RSV), Rabies virus, California encephalitis virus, Japanese encephalitis virus, Hantaan virus, Lymphocytic choriomeningitis virus, Coronavirus, Enterovirus, Rhinovirus, Poliovirus, Norovirus, Flavivirus, Dengue virus, West Nile virus, Yellow fever virus and varicella.

In an embodiment, the peptide antigen is derived from HPV. In an embodiment, the HPV peptide antigen is one that is associated with HPV-related cervical cancer or HPV-related head and neck cancer. In an embodiment, the peptide antigen is a peptide comprising the sequence RAHYNIVTF (HPV16E7 (H-2Db) peptide 49-57; R9F; SEQ ID NO: 9). In an embodiment, the peptide antigen is a peptide comprising the sequence YMLNLGPET (HPV Y9T peptide; SEQ ID NO: 10).

In an embodiment, the peptide antigen is derived from HIV. In an embodiment, the HIV peptide antigen may be derived from the V3 loop of HIV-1 gp120. In an embodiment, the HIV peptide antigen may be RGP10 (RGPGRAFVTI; SEQ ID NO: 11). RGP10 may be purchased from Genscript (Piscataway, N.J.). In another embodiment, the peptide antigen may be AMQ9 (AMQMLKETI; SEQ ID NO: 12). AMQ9 peptide is the immunodominant MHC class I epitope of gag for mice of the H-2Kd haplotype. AMQ9 may also be purchased from Genscript.

In an embodiment, the peptide antigen is derived from RSV. The RSV virion, a member of the genus Paramyxoviridae, is composed of a single strand of negative-sense RNA with 15,222 nucleotides. The nucleotides encode three transmembrane surface proteins (F, G and small hydrophobic protein or SH), two matrix proteins (M and M2), three nucleocapsid proteins (N, P and L), and two non-structural proteins (NS1 and NS2). In an embodiment, the peptide antigen may be derived from any one or more of the RSV proteins. In a particular embodiment, the peptide antigen may be derived from the SH protein of RSV or any other paramyxovirus, or a fragment thereof. The RSV peptide antigen may be any one or more of the RSV peptides described or disclosed in WO 2012/065997.

The SH protein, present in a number of paramyxoviruses (Collins 1990), is a transmembrane protein with an ectodomain or “extracellular” component. The human RSV SH protein contains 64 amino acids (Subgroup A) and 65 amino acids (Subgroup B) and is highly conserved.

Human RSV SH (SubgroupA):
(SEQ ID NO: 13)
MENTSITIEFSSKFWPYFTLIHMITTIISLLIIISIMIA
ILNKLCEYNVFHNKTFELPRARVNT
Human RSV SH (Subgroup B):
(SEQ ID NO: 14)
MGNTSITIEFTSKFWPYFTLIHMILTLISLLIIITIMIA
ILNKLSEHKTFCNKTLEQGQMYQINT

In an embodiment, the peptide antigen comprises or consists of the ectodomain of the SH protein (SHe) of a paramyxovirus, or a fragment or modified variant thereof. In an embodiment, SHe is derived from bovine RSV. In another embodiment, SHe is derived from a subgroup A human RSV strain or a subgroup B human RSV strain.

Subgroup A human RSV SHe (RSV SHe A):
(SEQ ID NO: 7)
NKLCEYNVFHNKTFELPRARVNT
Subgroup B human RSV SHe (RSV SHe B):
(SEQ ID NO: 8)
NKLSEHKTFCNKTLEQGQMYQINT

In an embodiment, the RSV peptide antigen may be in monomeric form, dimeric form, or another oligomeric form, or any combination thereof. In an embodiment, the peptide antigen comprising SHe A and/or SHe B is a monomer (e.g. a single polypeptide). In another embodiment, the peptide antigen comprising SHe A and/or SHe B is dimer (e.g. two separate polypeptides dimerized). Means of dimerization are known in the art. An exemplary procedure is to dissolve the RSV SHe peptide antigens in a mixture of 10% DMSO/0.5% acetic acid in water (w/w) and heat at 37° C. overnight.

In an embodiment, the peptide antigen derived from RSV may comprise or consist of any one or more of the following:

| Name | Sequence | SEQ ID NO |
|---|---|---|
| SheA | NKLCEYNVFHNKTFELPRARVNT | 7 |
| (monomer) | | |
| SheA (dimer) | 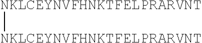 | 7 7 |
| SHeA | NKLSEYNVFHNKTFELPRARVNT | 15 |
| (C45S) | | |
| bSheA | NKLCDLNDHHTNSLDIRTRLRNDTQLITRAHEGSINQSSN | 16 |
| (monomer) | | |
| bSheA (dimer) | 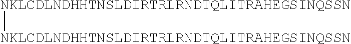 | 16 16 |
| bSHeA | NKLSDLNDHHTNSLDIRTRLRNDTQLITRAHEGSINQSSN | 17 |
| (C45S) | | |
| SheB | NKLSEHKTFCNKTLEQGQMYQINT | 8 |
| (monomer) | | |
| SheB (dimer) | 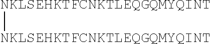 | 8 8 |
| SHeB | NKLSEHKTFSNKTLEQGQMYQINT | 18 |
| (C51S) | | |
| SHeB | NKLCEHKTFSNKTLEQGQMYQINT | 19 |
| (C45S) | | |
| SHeB (S45C) | 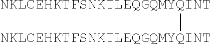 | 19 19 |
| L-SHe B (C51S) | 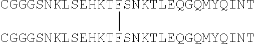 | 20 20 |

As described for example in WO 2012/065997, the SHe peptide antigen may be genetically or chemically linked to a carrier. Exemplary embodiments of carriers suitable for presentation of peptide antigens are known in the art, some of which are described in WO 2012/065997. In another embodiment, the SHe peptide antigen may be linked to a sized lipid vesicle particle as described herein or a structure formed therefrom or resulting therefrom as a result of the methods of manufacture.

In another embodiment, the peptide antigen is derived from an influenza virus. Influenza is a single-stranded RNA virus of the family Orthomyxoviridae and is often characterized based on two large glycoproteins on the outside of the viral particle, hemagglutinin (HA) and neuraminidase (NA). Numerous HA subtypes of influenza A have been identified (Kawaoka 1990; Webster 1983). In some embodiments, the antigen may be derived from the HA or NA glycoproteins. In a particular embodiment, the antigen may be recombinant HA antigen (H5N1, A/Vietnam/1203/2004; Protein Sciences; USA), such as derived from the sequence found under GenBank Accession number AY818135 or any suitable sequence variant thereof.

Bacteria, or parts thereof, from which a peptide antigen may be derived include for example, and without limitation, Anthrax (Bacillus anthracis), Brucella, Bordetella pertussis, Candida, Chlamydia pneumoniae, Chlamydia psittaci, Cholera, Clostridium botulinum, Coccidioides immitis, Cryptococcus, Diphtheria, Escherichia coli 0157: H7, Enterohemorrhagic Escherichia coli, Enterotoxigenic Escherichia coli, Haemophilus influenzae, Helicobacter pylori, Legionella, Leptospira, Listeria, Meningococcus, Mycoplasma pneumoniae, Mycobacterium, Pertussis, Pneumonia, Salmonella, Shigella, Staphylococcus, Streptococcus pneumoniae and Yersinia enterocolitica.

In an embodiment, the peptide antigen is derived from a Bacillus anthracis. Without limitation, the peptide antigen may for example be derived from anthrax recombinant protective antigen (rPA) (List Biological Laboratories, Inc.; Campbell, Calif.) or anthrax mutant recombinant protective antigen (mrPA). rPA has an approximate molecular weight of 83,000 daltons (Da) and corresponds a cell binding component of the three-protein exotoxin produced by Bacillus anthracis. The protective antigen mediates the entry of anthrax lethal factor and edema factor into the target cell. In some embodiments, the antigen may be derived from the sequence found under GenBank Accession number P13423, or any suitable sequence variant thereof.

Protozoa, or parts thereof, from which a peptide antigen may be derived include for example, and without limitation, the genus Plasmodium (Plasmodium falciparum, Plasmodium malariae, Plasmodium vivax, Plasmodium ovale or Plasmodium knowlesi), which causes malaria.

In an embodiment, the peptide antigen is derived from a Plasmodium species. For example, and without limitation, the peptide antigen may be derived from the circumsporozoite protein (CSP), which is a secreted protein of the sporozoite stage of the malaria parasite (Plasmodium sp.). The amino-acid sequence of CSP consists of an immunodominant central repeat region flanked by conserved motifs at the N- and C-termini that are implicated in protein processing as the parasite travels from the mosquito to the mammalian vector. The structure and function of CSP is highly conserved across the various strains of malaria that infect humans, non-human primates and rodents. In an embodiment, the peptide antigen derived from CSP is a malaria virus-like particle (VLP) antigen which comprises circumsporozoite T and B cell epitopes displayed on the woodchuck hepatitis virus core antigen.

In another embodiment, the peptide antigen may be derived from a cancer or tumor-associated protein, such as for example, a membrane surface-bound cancer antigen.

In an embodiment, the cancer may be one that is caused by a pathogen, such as a virus. Viruses linked to the development of cancer are known to the skilled person and include, but are not limited to, human papillomaviruses (HPV), John Cunningham virus (JCV), Human herpes virus 8, Epstein Barr Virus (EBV), Merkel cell polyomavirus, Hepatitis C Virus and Human T cell leukaemia virus-1. Thus, in an embodiment, the peptide antigen may be derived from a virus that is linked to the development of cancer.

In an embodiment, the peptide antigen is a cancer-associated antigen. Many cancer or tumor-associated proteins are known in the art such as for example, and without limitation, those described in WO 2016/176761. The methods, dried preparations, compositions, uses and kits disclosed herein may use or comprise any peptide antigen of a cancer-associated antigen, or a fragment or modified variant thereof.

In a particular embodiment, the peptide antigen is one or more survivin antigens.

Survivin, also called baculoviral inhibitor of apoptosis repeat-containing 5 (BIRC5), is a protein involved in the negative regulation of apoptosis. It has been classed as a member of the family of inhibitors of apoptosis proteins (IAPs). Survivin is a 16.5 kDa cytoplasmic protein containing a single BIR motif and a highly charged carboxy-terminal coiled region instead of a RING finger. The gene coding for survivin is nearly identical to the sequence of Effector Cell Protease Receptor-1 (EPR-1), but oriented in the opposite direction. The coding sequence for the survivin (Homo sapiens) is 429 nucleotides long including stop codons:

| SEQ ID NO: 21 | |
|---|---|
| atgggtgccc cgacgttgcc ccctgcctgg cagccctttc tcaaggacca ccgcatctct | 60 |
| acattcaaga actggccctt cttggagggc tgcgcctgca ccccggagcg gatggccgag | 120 |
| gctggcttca tccactgccc cactgagaac gagccagact tggcccagtg tttcttctgc | 180 |
| ttcaaggagc tggaaggctg ggagccagat gacgacccca tagaggaaca taaaaagcat | 240 |
| tcgtccggtt gcgctttcct ttctgtcaag aagcagtttg aagaattaac ccttggtgaa | 300 |
| tttttgaaac tggacagaga aagagccaag aacaaaattg caaaggaaac caacaataag | 360 |
| aagaaagaat ttgaggaaac tgcgaagaaa gtgcgccgtg ccatcgagca gctggctgcc | 420 |
| atggattga | 429 |

The encoded protein survivin (Homo sapiens) is 142 amino acids long:

```
SEQ ID NO: 22
Met Gly Ala Pro Thr Leu Pro Pro Ala Trp
1 5 10
Gln Pro Phe Leu Lys Asp His Arg Ile Ser
                                15 20
Thr Phe Lys Asn Trp Pro Phe Leu Glu Gly
                                25 30
Cys Ala Cys Thr Pro Glu Arg Met Ala Glu
                                35 40
Ala Gly Phe Ile His Cys Pro Thr Glu Asn
                                45 50
```

```
Glu Pro Asp Leu Ala Gln Cys Phe Phe Cys
                                55 60
Phe Lys Glu Leu Glu Gly Trp Glu Pro Asp
                                65 70
Asp Asp Pro Ile Glu Glu His Lys Lys His
                                75 80
Ser Ser Gly Cys Ala Phe Leu Ser Val Lys
                                85 90
Lys Gln Phe Glu Glu Leu Thr Leu Gly Glu
                        95 100
Phe Leu Lys Leu Asp Arg Glu Arg Ala Lys
                                105 110
Asn Lys Ile Ala Lys Glu Thr Asn Asn Lys
                                115 120
Lys Lys Glu Phe Glu Glu Thr Ala Lys Lys
                                125 130
Val Arg Arg Ala Ile Glu Gln Leu Ala Ala
                        135 140
Met Asp
```

In an embodiment, the peptide antigen is any peptide, polypeptide or variant thereof derived from a survivin protein, or a fragment thereof.

In an embodiment, the peptide antigen may be a survivin antigen, such as for example and without limitation, those disclosed in WO 2016/176761.

In an embodiment, the survivin peptide antigen may comprise the full length survivin polypeptide. Alternatively, the survivin peptide antigen may be a survivin peptide comprising a fragment of any length of the survivin protein. Exemplary embodiments include a survivin peptide that comprises at least 5, 6, 7, 8, 9, 10, 11, 12, 13, 14, 15, 16, 17, 18, 19 or 20 amino acid residues. In specific embodiments, the survivin peptide consists of a heptapeptide, an octapeptide, a nonapeptide, a decapeptide or an undecapeptide, consisting of 7, 8, 9, 10, 11 consecutive amino acid residues of the survivin protein (e.g. SEQ ID NO: 22), respectively. Particular embodiments of the survivin antigen include survivin peptides of about 9 or 10 amino acids.

Survivin peptide antigens also encompass variants and functional equivalents of natural survivin peptides. Variants or functional equivalents of a survivin peptide encompass peptides that exhibit amino acid sequences with differences as compared to the specific sequence of the survivin protein, such as one or more amino acid substitutions, deletions or additions, or any combination thereof. The difference may be measured as a reduction in identity as between the survivin protein sequence and the survivin peptide variant or survivin peptide functional equivalent.

In an embodiment, a vaccine composition of the invention may include any one or more of the survivin peptides, survivin peptide variants or survivin peptide functional equivalents disclosed in WO 2004/067023; WO 2006/081826 or WO 2016/176761.

In a particular embodiment, the survivin peptide antigen may be any one or more of:

(SEQ ID NO: 23)
i) FEELTLGEF [HLA-A1]
(SEQ ID NO: 1)
ii) FTELTLGEF [HLA-A1]
(SEQ ID NO: 24)
iii) LTLGEFLKL [HLA-A2]
(SEQ ID NO: 2)
iv) LMLGEFLKL [HLA-A2]
(SEQ ID NO: 25)
v) RISTFKNWPF [HLA-A3]
(SEQ ID NO: 6)
vi) RISTFKNWPK [HLA-A3]
(SEQ ID NO: 3)
vii) STFKNWPFL [HLA-A24]
(SEQ ID NO: 4)
viii) LPPAWQPFL [HLA-B7]

The above-listed survivin peptides represent, without limitation, exemplary MHC Class I restricted peptides. The specific MHC Class I HLA molecule to which each of the survivin peptides is believed to bind is shown on the right in square brackets.

In an embodiment, the methods, dried preparations, compositions, uses and kits disclosed herein use or comprise one or more of the following survivin peptide antigens:

(SEQ ID NO: 1)
i) FTELTLGEF [HLA-A1]
(SEQ ID NO: 2)
ii) LMLGEFLKL [HLA-A2]
(SEQ ID NO: 6)
iii) RISTFKNWPK [HLA-A3]
(SEQ ID NO: 3)
iv) STFKNWPFL [HLA-A24]
(SEQ ID NO: 4)
v) LPPAWQPFL [HLA-B7]

In an embodiment, the methods, dried preparations, compositions, uses and kits disclosed herein use or comprise all five of the survivin peptide antigens listed above.

In an embodiment, the peptide antigen is a self-antigen. As is well-known in the art, a self-antigen is an antigen that originates from within the body of a subject. The immune system is usually non-reactive against self-antigens under normal homeostatic conditions. These types of antigens therefore pose a difficulty in the development of targeted immune therapies. In an embodiment, the peptide antigen is a self-antigen or a fragment or modified variant thereof.

In an embodiment, the peptide antigen is a neoantigen. As used herein, the term “neoantigen” refers to a class of tumor antigens which arise from tumor-specific mutations in an expressed protein. The neoantigen can be derived from any cancer, tumor or cell thereof.

In the context of neoantigens, the term “derived from” as used herein encompasses, without limitation: a neoantigen that is isolated or obtained directly from an originating source (e.g. a subject); a synthetic or recombinantly generated neoantigen that is identical in sequence to a neoantigen from an originating source; or a neoantigen which is made from a neoantigen of an originating source or a fragment thereof.

The mutations in the expressed protein that create the neoantigen may be patient-specific. By “patient-specific”, it is meant that the mutation(s) are unique to an individual subject. However, it is possible that more than one subject will share the same mutation(s). Thus, a “patient-specific” mutation may be shared by a small or large sub-population of subjects.

The neoantigen may comprise one or more neoepitopes. As used herein, the term “epitope” refers to a peptide sequence which can be recognized by the immune system, specifically by antibodies, B cells or T cells. A “neoepitope” is an epitope of a neoantigen which comprises a tumor-specific mutation as compared to the native amino acid sequence. Generally, neoepitopes may be identified by screening neoantigens for anchor residues that have the potential to bind patient HLA. The neoepitopes are normally ranked using algorithms, such as NetMHC, that can predict peptide binding to HLA.

A “T-cell neoepitope” is to be understood as meaning a mutated peptide sequence which can be bound by the MHC molecules of class I or II in the form of a peptide-presenting MHC molecule or MHC complex. The T-cell neoepitope should typically be one that is amenable to recognition by T cell receptors so that a cell-mediated immune response can occur. A “B-cell neoepitope” is to be understood as meaning a mutated peptide sequence which can be recognized by B cells and/or by antibodies.

In some embodiments, at least one of the neoepitopes of the neoantigen is a patient-specific neoepitope. As used herein, by “patient-specific neoepitope”, it is meant that the mutation(s) in the neoepitope are unique to an individual subject. However, it is possible that more than one subject will share the same mutation(s). Thus, a “patient-specific neoepitope” may be shared by a small or large sub-population of subjects.

As is apparent from the above, neoantigens can comprise a diverse set of peptides that are unique to an individual. These peptides may have different solubility properties which would make them difficult to formulate in conventional types of vaccine formulations, such as aqueous buffer or emulsion type formulations. Additionally, there may be pre-existing tolerance to these peptides in the host from which they were derived. These aspects, among others, may cause the neoantigens to be weakly immunogenic. Therefore, it is important to deliver them in a composition that is capable of generating a robust immune response, as disclosed herein.

As used herein, by “weakly immunogenic” it is meant that in conventional vaccines (e.g. aqueous vaccines, emulsions, etc.), the neoantigens have little or no ability to induce, maintain and/or boost a neoantigen-specific immune response. In an embodiment, a weakly immunogenic neoantigen is one that has little or no ability to induce, maintain and/or boost a neoantigen-specific immune response after a single administration of the neoantigen.

In an embodiment, the neoantigen may be selected from mutated somatic proteins of a cancer using selection algorithms such as NetMHC which look for motifs predicted to bind to MHC class I and/or MHC class II proteins.

In an embodiment, the neoantigen may be derived from a mutated gene or protein that has previously been associated with cancer phenotypes, such as for example tumor suppressor genes (e.g. p53); DNA repair pathway proteins (e.g. BRCA2) and oncogenes. Exemplary embodiments of genes which often contain mutations giving rise to cancer phenotypes are described, for example, in Castle 2012. The skilled person will be well aware of other mutated genes and/or proteins associated with cancer, and these are available from other literature sources.

In some embodiments, the neoantigen may comprise or consist of the neoantigens disclosed by Castle 2012. Castle 2012 does not provide the actual sequences of the neoantigens, but does provide the gene ID and location of the mutated peptide from which the actual sequence can be identified using e.g. the PubMed database available online from the National Center for Biotechnology Information (NCBI).

In an embodiment, the neoantigen may be one or more of the Mut1-50 neoantigens disclosed in Table 1 of Castle 2012, or a neoantigen of the same or related protein (e.g. a human homologue). In an embodiment, the neoantigen may be selected from the neoantigen peptides listed in Table 5 herein, or a neoantigen of the same or related protein (e.g. a human homologue). In an embodiment, the neoantigen may be one or more of Mut25 (STANYNTSHLNNDVWQIFENPVDWKEK; SEQ ID NO: 26), Mut30 (PSKPSFQEFVDWENVSPELNSTDQPFL; SEQ ID NO: 27) and Mut44 (EFKHIKAFDRTFANNPGPMVVFATPGM; SEQ ID NO: 28), or a neoantigen of the same or related protein (e.g. a human homologue).

DNA or RNA Polynucleotides that Encodes a Polypeptide

In an embodiment, the one or more of the therapeutic agents may be a DNA polynucleotide or RNA polynucleotide that encodes a polypeptide. In an embodiment, the DNA or RNA polynucleotide encodes one or more of the peptide antigens described herein.

As used herein, the “DNA or RNA polynucleotide” encompasses a chain of nucleotides of any length (e.g. 9, 12, 15, 18, 21, 24, 27, 30, 60, 90, 120, 150, 300, 600, 1200, 1500 or more nucleotides) or number of strands (e.g. single-stranded or double-stranded). Polynucleotides may be DNA (e.g. genomic DNA, cDNA, plasmid DNA) or RNA (e.g. mRNA) or combinations thereof. The polynucleotide may be naturally occurring or synthetic (e.g. chemically synthesized). It is contemplated that the polynucleotide may contain modifications of one or more nitrogenous bases, pentose sugars or phosphate groups in the nucleotide chain. Such modifications are well-known in the art and may be for the purpose of e.g. improving stability, solubility or transcriptional/translational activity of the polynucleotide.

In an embodiment, the polynucleotide encodes a polypeptide to be expressed in vivo in a subject. The invention is not limited to the expression of any particular type of polypeptide.

The polynucleotide may be used in various forms. In an embodiment, a naked polynucleotide may be used, either in linear form, or inserted into a plasmid, such as an expression plasmid. In other embodiments, a live vector such as a viral vector or bacterial vector may be used.

Depending on the nature of the polynucleotide and the intended use, one or more regulatory sequences that aid in transcription of DNA into RNA and/or translation of RNA into a polypeptide may be present. For example, if it is intended or not required that the polynucleotide be transcribed or translated, such regulatory sequences may be absent. In some instances, such as in the case of a polynucleotide that is a messenger RNA (mRNA) molecule, regulatory sequences relating to the transcription process (e.g. a promoter) are not required, and protein expression may be effected in the absence of a promoter. The skilled artisan can include suitable regulatory sequences as the circumstances require.

In some embodiments, the polynucleotide is present in an expression cassette, in which it is operably linked to regulatory sequences that will permit the polynucleotide to be expressed in the subject. The choice of expression cassette depends on the subject as well as the features desired for the expressed polypeptide.

Typically, an expression cassette includes a promoter that is functional in the subject and can be constitutive or inducible; a ribosome binding site; a start codon (ATG) if necessary; the polynucleotide encoding the polypeptide of interest; a stop codon; and optionally a 3′terminal region (translation and/or transcription terminator). Additional sequences such as a region encoding a signal peptide may be included. The polynucleotide encoding the polypeptide of interest may be homologous or heterologous to any of the other regulatory sequences in the expression cassette. Sequences to be expressed together with the polypeptide of interest, such as a signal peptide encoding region, are typically located adjacent to the polynucleotide encoding the protein to be expressed and placed in proper reading frame. The open reading frame constituted by the polynucleotide encoding the protein to be expressed solely or together with any other sequence to be expressed (e.g. the signal peptide), is placed under the control of the promoter so that transcription and translation occur in the subject to which the composition is administered.

Promoters suitable for expression of polynucleotides in a wide range of host systems are well-known in the art. Promoters suitable for expression of polynucleotides in mammals include those that function constitutively, ubiquitously or tissue-specifically. Examples of non-tissue specific promoters include promoters of viral origin. Examples of viral promoters include Mouse Mammary Tumor Virus (MMTV) promoter, Human Immunodeficiency Virus Long Terminal Repeat (HIV LTR) promoter, Moloney virus, avian leukosis virus (ALV), Cytomegalovirus (CMV) immediate early promoter/enhancer, Rous Sarcoma Virus (RSV), adeno-associated virus (AAV) promoters; adenoviral promoters, and Epstein Barr Virus (EBV) promoters. Compatibility of viral promoters with certain polypeptides is a consideration since their combination may affect expression levels. It is possible to use synthetic promoter/enhancers to optimize expression (see e.g. US patent publication 2004/0171573).

An example of a tissue-specific promoter is the desmin promoter which drives expression in muscle cells (Li 1989; Li & Paulin 1991; and Li & Paulin 1993). Other examples include artificial promoters such as a synthetic muscle specific promoter and a chimeric muscle-specific/CMV promoter (Li 1999; Hagstrom 2000).

As noted above, the polynucleotide of interest, together with any necessary regulatory sequences, may be delivered naked, e.g. either alone or as part of a plasmid, or may be delivered in a viral or bacterial or bacterial vector.

Whether a plasmid-type vector, or a bacterial or viral vector is used, it may be desirable that the vector be unable to replicate or integrate substantially in the subject. Such vectors include those whose sequences are free of regions of substantial identity to the genome of the subject, as to minimize the risk of host-vector recombination. One way to do this is to use promoters not derived from the recipient genome to drive expression of the polypeptide of interest. For example, if the recipient is a mammal, the promoter is preferably non-mammalian derived though it should be able to function in mammalian cells, e.g. a viral promoter.

Viral vectors that may be used to deliver the polynucleotide include e.g. adenoviruses and poxviruses. Useful bacterial vectors include e.g. Shigella, Salmonella, Vibrio cholerae, Lactobacillus, Bacille bilie de Calmette-Guerin (BCG), and Streptococcus.

An example of an adenovirus vector, as well as a method for constructing an adenovirus vector capable of expressing a polynucleotide, is described in U.S. Pat. No. 4,920,209. Poxvirus vectors include vaccinia and canary pox virus, described in U.S. Pat. Nos. 4,722,848 and 5,364,773, respectively. Also see, e.g., Tartaglia 1992 for a description of a vaccinia virus vector and Taylor 1995 for a reference of a canary pox.

Poxvirus vectors capable of expressing a polynucleotide of interest may be obtained by homologous recombination as described in Kieny 1984, so that the polynucleotide is inserted in the viral genome under appropriate conditions for expression in mammalian cells.

With respect to bacterial vectors, non-toxicogenic Vibrio cholerae mutant strains that are useful for expressing a foreign polynucleotide in a host are known. Mekalanos 1983 and U.S. Pat. No. 4,882,278 describe strains which have a substantial amount of the coding sequence of each of the two ctxA alleles deleted so that no functional cholerae toxin is produced. WO 92/11354 describes a strain in which the irgA locus is inactivated by mutation; this mutation can be combined in a single strain with ctxA mutations. WO 94/01533 describes a deletion mutant lacking functional ctxA and attRSl DNA sequences. These mutant strains are genetically engineered to express heterologous proteins, as described in WO 94/19482.

Attenuated Salmonella typhimurium strains, genetically engineered for recombinant expression of heterologous proteins are described in Nakayama 1988 and WO 92/11361.

Other bacterial strains which may be used as vectors to express a foreign protein in a subject are described for Shigella flexneri in High 1992 and Sizemore 1995; for Streptococcus gordonii in Medaglini 1995; and for Bacille Calmette Guerin in Flynn 1994, WO 88/06626, WO 90/00594, WO 91/13157, WO 92/01796, and WO 92/21376.

In bacterial vectors, the polynucleotide of interest may be inserted into the bacterial genome or remain in a free state as part of a plasmid.

Hormones

In an embodiment, one or more of the therapeutic agents may be a hormone, or a fragment, analog or variant thereof. The hormone, fragment, analog or variant thereof may be obtained from a natural source or be synthetically prepared.

Exemplary hormones include, without limitation, amylin, insulin, glucagon, erythropoietin (EPO), glucagon-like peptide-1 (GLP-1), melanocyte stimulating hormone (MSH), parathyroid hormone (PTH), thyroid-stimulating hormone, growth hormone (GH), growth hormone releasing hormone (GHRH), calcitonin, somatostatin, somatomedin (insulin-like growth factor), interleukins (e.g. interleukins 1-17), granulocyte/monocyte colony stimulating factor (GM-CSF), granulocyte colony stimulating factor (G-CSF), testosterone, interferons (e.g. interferon-alfa or -gamma), leptin, luteinizing hormone (LH), follicle-stimulating hormone (FSH), human chorionic gonadotropin (hCG), enkephalin, basic fibroblast growth factor (bFGF), luteinizing hormone, gonadotropin releasing hormone (GnRH), brain-derived natriuretic peptide (BNP), tissue plasminogen activator (TPA), oxytocin, relaxin, steroids (e.g. androgens, estrogens, glucocorticoids, progestogens and secosteroids) and analogs and combinations thereof.

Cytokines

In an embodiment, one or more of the therapeutic agents may be a cytokine, or a fragment, analog or variant thereof. The cytokine, fragment, analog or variant thereof may be obtained from a natural source or be synthetically prepared.

Exemplary cytokines include, without limitation, chemokines, interferons, interleukins, lymphokines and tumor necrosis factors, and analogs thereof.

Allergens

In an embodiment, one or more of the therapeutic agents may be an allergen, or a fragment, analog or variant thereof. The allergen, fragment, analog or variant thereof may be obtained from a natural source or be synthetically prepared.

An “allergen”, as used herein, refers to any substance that can cause an allergy. The allergen may be derived from, without limitation, cells, cell extracts, proteins, polypeptides, peptides, polysaccharides, polysaccharide conjugates, peptide and non-peptide mimics of polysaccharides and other molecules, small molecules, lipids, glycolipids, and carbohydrates of plants, animals, fungi, insects, food, drugs, dust, and mites. Allergens include but are not limited to environmental aeroallergens; plant pollens (e.g. ragweed/hayfever); weed pollen allergens; grass pollen allergens; Johnson grass; tree pollen allergens; ryegrass; arachnid allergens (e.g. house dust mite allergens); storage mite allergens; Japanese cedar pollen/hay fever; mold/fungal spore allergens; animal allergens (e.g. dog, guinea pig, hamster, gerbil, rat, mouse, etc., allergens); food allergens (e.g. crustaceans; nuts; citrus fruits; flour; coffee); insect allergens (e.g. fleas, cockroach); venoms: (Hymenoptera, yellow jacket, honey bee, wasp, hornet, fire ant); bacterial allergens (e.g. streptococcal antigens; parasite allergens such as Ascaris antigen); viral allergens; drug allergens (e.g. penicillin); hormones (e.g. insulin); enzymes (e.g. streptokinase); and drugs or chemicals capable of acting as incomplete antigens or haptens (e.g. the acid anhydrides and the isocyanates).

Catalytic DNA or RNA

In an embodiment, one or more of the therapeutic agents may be a catalytic DNA (deoxyribozyme) or a catalytic RNA (ribozyme).

As used herein, the term “catalytic DNA” refers to any DNA molecule with enzymatic activity. In an embodiment, the catalytic DNA is a single-stranded DNA molecule. In an embodiment, the catalytic DNA is synthetically produced as opposed to naturally occurring.

The catalytic DNA may perform one or more chemical reactions. In an embodiment, the catalytic DNA is a ribonuclease, whereby the catalytic DNA catalyzes the cleavage of ribonucleotide phosphodiester bonds. In another embodiment, the catalytic DNA is a DNA ligase, whereby the catalytic DNA catalyzes the joining of two polynucleotide molecules by forming a new bond. In other embodiments, the catalytic DNA can catalyze DNA phosphorylation, DNA adenylation, DNA deglycosylation, porphyrin metalation, thymine dimer photoreversion, or DNA cleavage.

As used herein, the term “catalytic RNA” refers to any RNA molecule with enzymatic activity. Catalytic RNAs are involved in a number of biological processes, including RNA processing and protein synthesis. In an embodiment, the catalytic RNA is a naturally occurring RNA. In an embodiment, the catalytic RNA is synthetically produced.

Antisense RNA

In an embodiment, one or more of the therapeutic agents may be an antisense RNA.

As used herein, an “antisense RNA” is any single-stranded RNA that is complementary to a messenger RNA (mRNA). The antisense RNA may exhibit 100% complementarity to the mRNA or less than 100% complementarity so long as the antisense RNA is still able to inhibit translation of the mRNA by base pairing to it, thereby obstructing the translation machinery.

In an embodiment, the antisense RNA is highly structured, comprised of one or more stem-and-loop secondary structures, flanked or separated by single-stranded (unpaired) regions. In some embodiments, tertiary structures, such as pseudoknots, may form between two or more secondary structural elements.

Interfering RNA and Antagomirs

In an embodiment, one or more of the therapeutic agents may be an interfering RNA, such as a small interfering RNA (siRNA), a microRNA (miRNA) or a small hairpin RNA (shRNA).

RNA interference (RNAi) is a biological process in which RNA molecules inhibit gene expression or translation, by neutralizing targeted mRNA molecules. Two types of small ribonucleic acid (RNA) molecules—microRNA (miRNA) and small interfering RNA (siRNA)—are central to RNA interference.

siRNA is a class of double-stranded RNA molecules that are typically 20-25 base pairs in length. It interferes with the expression of specific genes with complementary nucleotide sequences by degrading mRNA after transcription, thereby preventing translation. The natural structure of siRNA is typically a short 20-25 double-stranded RNA with two overhanging nucleotides on each end. The Dicer enzyme catalyzes production of siRNAs from long dsRNAs and small hairpin RNAs (shRNA). shRNA is an artificial RNA molecule with a tight hairpin turn. The design and production of siRNA molecules, and mechanisms of action, are known in the art.

miRNAs resemble siRNAs, except miRNAs derive from regions of RNA transcripts that fold back on themselves to form short hairpins, whereas siRNAs derive from longer double-stranded RNA.

In an embodiment, the therapeutic agent may be any one or more of these interfering RNAs (siRNA, miRNA or shRNA). The interfering RNA should be one which is capable of decreasing or silencing (preventing) the expression of a gene/mRNA of its endogenous cellular counterpart. In an embodiment, the interfering RNA derived from a naturally occurring interfering RNA. In an embodiment, the interfering RNA is synthetically produced.

In an embodiment, the therapeutic agent may be an antagomir. Antagomirs (also known as anti-miRs or blockmirs) are synthetically engineered oligonucleotides that silence endogenous miRNA. It is unclear how antagomirization (the process by which an antagomir inhibits miRNA activity) operates, but it is believed to inhibit by irreversibly binding the miRNA. Because of the promiscuity of microRNAs, antagomirs could affect the regulation of many different mRNA molecules. Antagomirs are designed to have a sequence that is complementary to an mRNA sequence that serves as a binding site for microRNA.

Drugs

In an embodiment, one or more of the therapeutic agents is a drug, i.e. a chemical substance used to treat, cure, prevent or diagnose a disease, disorder or condition.

In an embodiment, and without limitation, exemplary drugs include immunomodulatory agents (immunostimulants and immunosuppressives), an immune response checkpoint molecule, antipyretics, analgesics, anti-migraine agents, anti-coagulant agents, anti-emetic agents, anti-inflammatory agents, antiviral agents, antibacterial agents, anti-fungal agents, cardiovascular agents, central nervous system agents, anti-hypertensive and vasodilator agents, sedatives, narcotic agonists, chelating agents, anti-diuretic agents, and anti-cancer agents, anti-neoplastic agents. Examples include the following:

In an embodiment, the drug is a small molecule drug. As used herein, the term “small molecule drug” refers an organic compound that may be used to treat, cure, prevent or diagnose a disease, disorder or condition.

The term “small molecule” is understood to refer to a low molecular weight compound which may be synthetically produced or obtained from natural sources and typically has a molecular weight of less than 2000 Da, less than 1000 Da or less than 600 Da. In a particular embodiment, the small molecule has a molecular weight of less than 900 Da, which allows for the possibility to rapidly diffuse across cell membranes. More particularly the small molecule has a molecular weight of less than 600 Da, and even more particularly less than 500 Da.

In an embodiment, the small molecule drug has a molecule weight of between about 100 Da to about 2000 Da; about 100 Da to about 1500 Da; about 100 Da to about 1000 Da; about 100 Da to about 900 Da; about 100 Da to about 800 Da; about 100 Da to about 700 Da; about 100 Da to about 600 Da; or about 100 Da to about 500 Da. In an embodiment, the small molecule drug has a molecular weight of about 100 Da, about 150 Da, about 200 Da, about 250 Da, about 300 Da, about 350 Da, about 400 Da, about 450 Da, about 500 Da, about 550 Da, about 600 Da, about 650 Da, about 700 Da, about 750 Da, about 800 Da, about 850 Da, about 900 Da, about 950 Da or about 1000 Da. In an embodiment, the small molecule drug may have a size on the order of 1 nm.

In an embodiment, the small molecule drug is one or more of: Epacadostat, Rapamycin, Doxorubicin, Valproic acid, Mitoxantrone, Vorinostat, Cyclophosphamide, Irinotecan, Cisplatin or Methotrexate. In a particular embodiment, the small molecule drug is Cyclophosphamide.

In an embodiment, the small molecule drug is an agent that interferes with DNA replication. As used herein, the expression “interferes with DNA replication” is intended to encompass any action that prevents, inhibits or delays the biological process of copying (i.e., replicating) the DNA of a cell. The skilled person will appreciate that there exist various mechanisms for preventing, inhibiting or delaying DNA replication, such as for example DNA cross-linking, methylation of DNA, base substitution, etc. The present disclosure encompasses the use of any agent that interferes with DNA replication by any means known in the art. Exemplary, non-limiting embodiments of such agents are described, for example, in WO2014/153636 and/or in PCT/CA2017/050539. In an embodiment, the agent that interferes with DNA replication is an alkylating agent, such as for example a nitrogen mustard alkylating agent. In an embodiment, the agent that interferes with DNA replication is Cyclophosphamide.

In an embodiment, the small molecule drug is an immune response checkpoint inhibitor. As used herein, an “immune response checkpoint inhibitor” refers to any compound or molecule that totally or partially reduces, inhibits, interferes with or modulates one or more checkpoint proteins. Checkpoint proteins regulate T-cell activation or function. Numerous checkpoint proteins are known, such as for example CTLA-4 and its ligands CD80 and CD86; and PD-1 and its ligands PD-L1 and PD-L2. Checkpoint proteins are responsible for co-stimulatory or inhibitory interactions of T-cell responses. Checkpoint proteins regulate and maintain self-tolerance and the duration and amplitude of physiological immune responses. Herein, the term “immune response checkpoint inhibitor” may be used interchangeably with “checkpoint inhibitor”. Exemplary non-limiting embodiments of checkpoint inhibitors are hereinafter described.

In an embodiment, the immune response checkpoint inhibitor is an inhibitor of Programmed Death-Ligand 1 (PD-L1, also known as B7-H1, CD274), Programmed Death 1 (PD-1, CD279), CTLA-4 (CD154), PD-L2 (B7-DC, CD273), LAG3 (CD223), TIM3 (HAVCR2, CD366), 41BB (CD137), 2B4, A2aR, B7H1, B7H3, B7H4, BTLA, CD2, CD27, CD28, CD30, CD40, CD70, CD80, CD86, CD160, CD226, CD276, DR3, GAL9, GITR, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGIT, VISTA, VTCN1, or any combination thereof.

In an embodiment, the immune response checkpoint inhibitor is an inhibitor of PD-L1, PD-1, CTLA-4 or any combination thereof.

In an embodiment, the drug is a biologic drug. As used herein, a “biologic drug” is any pharmaceutical drug product manufactured in, extracted from, or semisynthesized from biological sources. In an embodiment, the biologic drug is a blood component, a cell, a cellular component, an allergen, an antibody, a gene or fragment thereof, a tissue, a tissue component, or a recombinant protein.

Antibodies

In an embodiment, one or more of the therapeutic agents is an antibody, an antigen binding fragment thereof or a derivative thereof.

“Antibody” as used herein means an antibody of classes IgG, IgM, IgA, IgD or IgE, or fragments, or derivatives thereof, including Fab, F(ab′)2, Fd, and single chain antibodies, diabodies, bispecific antibodies, bifunctional antibodies and derivatives thereof. The antibody can be an antibody isolated from the serum sample of mammal, a monoclonal antibody, a polyclonal antibody, an affinity purified antibody, or mixtures thereof which exhibit sufficient binding specificity to a desired epitope or a sequence derived therefrom.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

As used herein, the term “antigen-binding fragment” refers to any fragment or portion of an antibody, or variant thereof, that remains capable of binding a specific target antigen of the full length antibody. In an embodiment, the antigen-binding fragment comprises the heavy chain variable and/or light chain variable region of the antibody.

In an embodiment, the antibody can be an anti-PD-1 antibody, a variant thereof or an antigen-binding fragment thereof, or a combination thereof. In an embodiment, the PD-1 antibody may be Nivolumab (Opdivo™). In an embodiment, the PD-1 antibody may be pembrolizumab (Keytruda™).

In other embodiments, without limitation, the antibody may be an anti-PD1 or anti-PDL1 antibody, such as for example those disclosed in WO 2015/103602. For example, in an embodiment, the anti-PD-1 antibody or anti-PD-LI antibody may be selected from: nivolumab, pembrolizumab, pidilizumab, BMS-936559 (see ClinicalTrials.gov; Identifier NCT02028403), MPDL3280A (Roche, see ClinicalTrials.gov; Identifier NCT02008227), MDX1105-01 (Bristol Myers Squibb, see ClinicalTrials.gov; Identifier NCT00729664), MEDI4736 (MedImmune, see ClinicalTrials.gov; Identifier NCT01693562), and MK-3475 (Merck, see ClinicalTrials.gov; Identifier NCT02129556). In an embodiment, the anti-PD-1 antibody may be RMP1-4 or J43 (BioXCell) or a human or humanized counterpart thereof.

In an embodiment, the antibody is an anti-CTL4 antibody, a variant thereof or an antigen-binding fragment thereof, or a combination thereof. The anti-CTL4 antibody can inhibit CTL4 activity, thereby inducing, eliciting, or enhancing immune responses. In an embodiment, the anti-CTLA-4 antibody may be ipilimumab (Bristol-Myers Squibb) or BN13 (BioXCell). In another embodiment, the anti-CTLA-4 antibody may be UC10-4F10-11, 9D9 or 9H10 (BioXCell) or a human or humanized counterpart thereof.

The amount of any specific therapeutic agent may depend on the type of the therapeutic agent (e.g. peptide antigen, small molecule drug, antibody, etc.). One skilled in the art can readily determine the amount of therapeutic agent needed in a particular application by empirical testing.

T-Helper Epitopes

In some embodiments, one or more T-helper epitopes may be used in the methods, dried preparations, compositions, uses or kits disclosed herein. In an embodiment, a T-helper epitope is used when at least one therapeutic agent is an antigen.

T-helper epitopes are a sequence of amino acids (natural or non-natural amino acids) that have T-helper activity. T-helper epitopes are recognised by T-helper lymphocytes, which play an important role in establishing and maximising the capabilities of the immune system, and are involved in activating and directing other immune cells, such as for example cytotoxic T lymphocytes. A T-helper epitope can consist of a continuous or discontinuous epitope. Hence not every amino acid of a T-helper is necessarily part of the epitope.

Accordingly, T-helper epitopes, including analogs and segments of T-helper epitopes, are capable of enhancing or stimulating an immune response. Immunodominant T-helper epitopes are broadly reactive in animal and human populations with widely divergent MHC types (Celis 1988, Demotz 1989, Chong 1992). The T-helper domain of the subject peptides may have from about 10 to about 50 amino acids, and more particularly about 10 to about 30 amino acids. When multiple T-helper epitopes are present, then each T-helper epitope acts independently.

In another embodiment, the T-helper epitope may be a T-helper epitope analog or a T-helper segment. T-helper epitope analogs may include substitutions, deletions and insertions of from one to about 10 amino acid residues in the T-helper epitope. T-helper segments are contiguous portions of a T-helper epitope that are sufficient to enhance or stimulate an immune response. An example of T-helper segments is a series of overlapping peptides that are derived from a single longer peptide.

In some embodiments, the T-helper epitope may form part of a peptide antigen described herein. In particular, if the peptide antigen is of sufficient size, it may contain an epitope that functions as a T-helper epitope. In other embodiments, the T-helper epitope is a separate molecule from the peptide antigen. In other embodiments, the T-helper epitope may be fused to the peptide antigen.

In a particular embodiment, the T-helper epitope may be the modified Tetanus toxin peptide A16L (amino acids 830 to 844; AQYIKANSKFIGITEL; SEQ ID NO: 5), with an alanine residue added to its amino terminus to enhance stability (Slingluff 2001).

Other sources of T-helper epitopes which may be used include, for example, hepatitis B surface antigen helper T cell epitopes, pertussis toxin helper T cell epitopes, measles virus F protein helper T cell epitope, Chlamydia trachomitis major outer membrane protein helper T cell epitope, diphtheria toxin helper T cell epitopes, Plasmodium falciparum circumsporozoite helper T cell epitopes, Schistosoma mansoni triose phosphate isomerase helper T cell epitopes, Escherichia coli TraT helper T cell epitopes and immune-enhancing analogs and segments of any of these T-helper epitopes.

In some embodiments, the T-helper epitope may be a universal T-helper epitope. A universal T-helper epitope as used herein refers to a peptide or other immunogenic molecule, or a fragment thereof, that binds to a multiplicity of MHC class II molecules in a manner that activates T cell function in a class II (CD4+ T cells)-restricted manner. An example of a universal T-helper epitope is PADRE (pan-DR epitope) comprising the peptide sequence AKXVAAWTLKAAA, wherein X may be cyclohexylalanyl (SEQ ID NO: 29). PADRE specifically has a CD4+T-helper epitope, that is, it stimulates induction of a PADRE-specific CD4+T-helper response.

In addition to the modified tetanus toxin peptide A16L mentioned earlier, Tetanus toxoid has other T-helper epitopes that work in the similar manner as PADRE. Tetanus and diphtheria toxins have universal epitopes for human CD4+ cells (Diethelm-Okita 2000). In another embodiment, the T-helper epitope may be a tetanus toxoid peptide such as F21E comprising the peptide sequence FNNFTVSFWLRVPKVSASHLE (amino acids 947 to 967; SEQ ID NO: 30).

Many other T-helper epitopes are known in the art, and any of these T-helper epitopes may be used in the practice of the methods, dried preparations, compositions, uses and kits disclosed herein.

In an embodiment, the dried preparations or compositions disclosed herein comprise a single type T-helper epitope. In another embodiment, the dried preparations or compositions disclosed herein comprise multiple different types of T-helper epitopes (e.g. 1, 2, 3, 4 or 5 different T-helper epitopes).

In an embodiment, the dried preparations or compositions disclosed herein do not comprise a T-helper epitope. For example, such may be the case when the therapeutic agent is not an antigen.

The amount of T-helper epitope used may depend on the type(s) and amount of therapeutic agent and on the type of T-helper epitope. One skilled in the art can readily determine the amount of T-helper epitope needed in a particular application by empirical testing.

Adjuvants

In some embodiments, one or more adjuvants may be used in the methods, dried preparations, compositions, uses or kits disclosed herein.

A large number of adjuvants have been described and are known to those skilled in the art. Exemplary adjuvants include, without limitation, alum, other compounds of aluminum, Bacillus of Calmette and Guerin (BCG), TiterMax™, Ribi™, Freund's Complete Adjuvant (FCA), CpG-containing oligodeoxynucleotides (CpG ODN), lipid A mimics or analogs thereof, lipopeptides and polyI:C polynucleotides.

In an embodiment, the adjuvant is a CpG ODN. CpG ODNs are DNA molecules that contain one or more unmethylated CpG motifs (consisting of a central unmethylated CG dinucleotide plus flanking regions). An exemplary CpG ODN is 5′-TCCATGACGTTCCTGACGTT-3′ (SEQ ID NO: 31). The skilled person can readily select other appropriate CpG ODNs on the basis of the target species and efficacy.

In an embodiment, the adjuvant is a polyI:C polynucleotide.

PolyI:C polynucleotides are polynucleotide molecules (either RNA or DNA or a combination of DNA and RNA) containing inosinic acid residues (I) and cytidylic acid residues (C), and which induce the production of inflammatory cytokines, such as interferon. In an embodiment, the polyI:C polynucleotide is double-stranded. In such embodiments, they may be composed of one strand consisting entirely of cytosine-containing nucleotides and one strand consisting entirely of inosine-containing nucleotides, although other configurations are possible. For instance, each strand may contain both cytosine-containing and inosine-containing nucleotides. In some instances, either or both strands may additionally contain one or more non-cytosine or non-inosine nucleotides.

It has been reported that polyI:C can be segmented every 16 residues without an effect on its interferon activating potential (Bobst 1981). Furthermore, the interferon inducing potential of a polyI:C molecule mismatched by introducing a uridine residue every 12 repeating cytidylic acid residues (Hendrix 1993), suggests that a minimal double stranded polyI:C molecule of 12 residues is sufficient to promote interferon production. Others have also suggested that regions as small as 6-12 residues, which correspond to 0.5-1 helical turn of the double stranded polynucleotide, are capable of triggering the induction process (Greene 1978). If synthetically made, polyI:C polynucleotides are typically about 20 or more residues in length (commonly 22, 24, 26, 28 or 30 residues in length). If semi-synthetically made (e.g. using an enzyme), the length of the strand may be 500, 1000 or more residues.

Accordingly, as used herein, a “polyI:C”, “polyI:C polynucleotide” or “polyI:C polynucleotide adjuvant” is a double- or single-stranded polynucleotide molecule (RNA or DNA or a combination of DNA and RNA), each strand of which contains at least 6 contiguous inosinic or cytidylic acid residues, or 6 contiguous residues selected from inosinic acid and cytidylic acid in any order (e.g. IICIIC or ICICIC), and which is capable of inducing or enhancing the production of at least one inflammatory cytokine, such as interferon, in a mammalian subject. PolyI:C polynucleotides will typically have a length of about 8, 10, 12, 14, 16, 18, 20, 22, 24, 25, 26, 28, 30, 35, 40, 45, 50, 55, 60, 65, 70, 75, 80, 85, 90, 95, 100, 150, 200, 250, 300, 500, 1000 or more residues. Preferred polyI:C polynucleotides may have a minimum length of about 6, 8, 10, 12, 14, 16, 18, 20, 22, 24, 26, 28, or 30 nucleotides and a maximum length of about 1000, 500, 300, 200, 100, 90, 80, 70, 60, 50, 45 or 40 nucleotides.

Each strand of a double-stranded polyI:C polynucleotide may be a homopolymer of inosinic or cytidylic acid residues, or each strand may be a heteropolymer containing both inosinic and cytidylic acid residues. In either case, the polymer may be interrupted by one or more non-inosinic or non-cytidylic acid residues (e.g. uridine), provided there is at least one contiguous region of 6 I, 6 C or 6 I/C residues as described above. Typically, each strand of a polyI:C polynucleotide will contain no more than 1 non-I/C residue per 6 I/C residues, more preferably, no more than 1 non-I/C residue per every 8, 10, 12, 14, 16, 18, 20, 22, 24, 26, 28 or 30 I/C residues.

The inosinic acid or cytidylic acid (or other) residues in the polyI:C polynucleotide may be derivatized or modified as is known in the art, provided the ability of the polyI:C polynucleotide to promote the production of an inflammatory cytokine, such as interferon, is retained. Non-limiting examples of derivatives or modifications include e.g. azido modifications, fluoro modifications, or the use of thioester (or similar) linkages instead of natural phosphodiester linkages to enhance stability in vivo. The polyI:C polynucleotide may also be modified to e.g. enhance its resistance to degradation in vivo by e.g. complexing the molecule with positively charged poly-lysine and carboxymethylcellulose, or with a positively charged synthetic peptide.

In an embodiment, the polyI:C polynucleotide may be a single-stranded molecule containing inosinic acid residues (I) and cytidylic acid residues (C). As an example, and without limitation, the single-stranded polyI:C may be a sequence of repeating dIdC. In a particular embodiment, the sequence of the single-stranded polyI:C may be a 26-mer sequence of (IC)₁₃, i.e. ICICICICICICICICICICICICIC (SEQ ID NO: 32). As the skilled person will appreciate, due to their nature (e.g. complementarity), it is anticipated that these single-stranded molecules of repeating dIdC would naturally form homodimers, so they are conceptually similar to polyI/polyC dimers.

In an embodiment, the polyI:C polynucleotide adjuvant is a traditional form of polyI:C with an approximate molecular weight of 989,486 Daltons, containing a mixture of varying strand lengths of polyI and polyC of several hundred base pairs (Thermo Scientific; USA).

In an embodiment, the adjuvant may be one that activates or increases the activity of TLR2. As used herein, an adjuvant which “activates” or “increases the activity” of a TLR2 includes any adjuvant, in some embodiments a lipid-based adjuvant, which acts as a TLR2 agonist. Further, activating or increasing the activity of TLR2 encompasses its activation in any monomeric, homodimeric or heterodimeric form, and particularly includes the activation of TLR2 as a heterodimer with TLR1 or TLR6 (i.e. TLR1/2 or TLR2/6). Exemplary embodiments of an adjuvant that activates or increases the activity of TLR2 include lipid-based adjuvants, such as those described in WO2013/049941.

In an embodiment, the adjuvant may be a lipid-based adjuvant, such as disclosed for example in WO2013/049941. In an embodiment, the lipid-based adjuvant is one that comprises a palmitic acid moiety such as dipalmitoyl-S-glyceryl-cysteine (PAM₂Cys) or tripalmitoyl-S-glyceryl-cysteine (PAM₃Cys). In an embodiment, the adjuvant is a lipopeptide. Exemplary lipopeptides include, without limitation, PAM₂Cys-Ser-(Lys)4 (SEQ ID NO: 33) or PAM₃Cys-Ser-(Lys)4 (SEQ ID NO: 33).

In an embodiment, the adjuvant is PAM₃Cys-SKKKK (EMC Microcollections, Germany; SEQ ID NO: 33) or a variant, homolog and analog thereof. The PAM₂ family of lipopeptides has been shown to be an effective alternative to the PAM₃ family of lipopeptides.

In an embodiment, the adjuvant may be a lipid A mimic or analog adjuvant, such as for example those disclosed in WO2016/109880 and the references cited therein. In a particular embodiment, the adjuvant may be JL-265 or JL-266 as disclosed in WO2016/109880.

In an embodiment, a combination of a polyI:C polynucleotide adjuvant and a lipid-based adjuvant may be used, such as described in the adjuvanting system disclosed in WO2017/083963.

Further examples of adjuvants that may be used include, without limitation, chemokines, colony stimulating factors, cytokines, 1018 ISS, aluminum salts, Amplivax, AS04, AS15, ABM2, Adjumer, Algammulin, AS01B, AS02 (SBASA), ASO2A, BCG, Calcitriol, Chitosan, Cholera toxin, CP-870,893, CpG, polyI:C, CyaA, DETOX (Ribi Immunochemicals), Dimethyldioctadecylammonium bromide (DDA), Dibutyl phthalate (DBP), dSLIM, Gamma inulin, GM-CSF, GMDP, Glycerol, IC30, IC31, Imiquimod, ImuFact IMP321, IS Patch, ISCOM, ISCOMATRIX, JuvImmune, LipoVac, LPS, lipid core protein, MF59, monophosphoryl lipid A and analogs or mimics thereof, Montanide® IMS1312, Montanide® based adjuvants (e.g. Montanide ISA-51, -50 and -70), OK-432, OM-174, OM-197-MP-EC, ONTAK, PepTel vector system, other palmitoyl based molecules, PLG microparticles, resiquimod, squalene, SLR172, YF-17 DBCG, QS21, QuilA, P1005, Poloxamer, Saponin, synthetic polynucleotides, Zymosan, pertussis toxin.

In an embodiment, at least one of the therapeutic agents may be coupled to at least one of the adjuvants. In an embodiment, the adjuvant is not coupled to any of the therapeutic agents.

The amount of adjuvant used may depend on the type(s) and amount of therapeutic agent and on the type of adjuvant. One skilled in the art can readily determine the amount of adjuvant needed in a particular application by empirical testing.

Surfactants

In an embodiment, the compositions disclosed herein may comprise one or more surfactants. The surfactant may be a single agent or a mixture of agents. The surfactant(s) should be pharmaceutically and/or immunologically acceptable.

In some embodiments, a surfactant may be used to assist in stabilizing the lipid-based structures having a single layer lipid assembly, therapeutic agents and/or other components (e.g. adjuvant and/or T-helper epitope) in the hydrophobic carrier. The use of a surfactant may, for example, promote more even distribution of the mixture of these components by reducing surface tensions. In an embodiment, a surfactant may be used when the compositions disclosed herein are to contain several different therapeutic agents (e.g. five or more different peptide antigens) or a relatively high concentration of therapeutic agent (e.g. ≥5 mg/mg total of therapeutic agent).

The surfactant may be amphipathic and therefore, the surfactant may include a broad range of compounds. Examples of surfactants which may be used include polysorbates, which are oily liquids derived from polyethylene glycolyated sorbital, and sorbitan esters. Polysorbates may include, for example, sorbitan monooleate. Typical surfactants are well-known in the art and include, without limitation, mannide oleate (Arlacel™ A), lecithin, Tween™ 80, Spans™ 20, 80, 83 and 85. In an embodiment, the surfactant for use in the compositions may be mannide oleate. In an embodiment, the surfactant for use in the compositions may be Span80.

The surfactant is generally pre-mixed with the hydrophobic carrier. In some embodiments, a hydrophobic carrier which already contains a surfactant may be used. For example, a hydrophobic carrier such Montanide™ ISA 51 already contains the surfactant mannide oleate. In other embodiments, the hydrophobic carrier may be mixed with a surfactant before combining with the other components (e.g. the dried lipid/therapeutic agent preparation).

The surfactant is used in an amount effective to promote even distribution of the dried preparation in the hydrophobic carrier and/or to assist in the formation of the single layer assembly of the lipid-based structures. Typically, the volume ratio (v/v) of hydrophobic carrier to surfactant is in the range of about 4:1 to about 15:1.

In an embodiment, the compositions do not contain a surfactant. In such embodiments, the small uniform size of the sized lipid vesicle particles may permit the lipids to easily rearrange to form the lipid-based structures having a single layer lipid assembly in the presence of the therapeutic agents and/or other components (e.g. adjuvant and/or T-helper epitope) in the hydrophobic carrier. Thus, in such embodiments, a surfactant is not required.

### EMBODIMENTS

Particular embodiments of the invention include, without limitation, the following:

(1) A method for preparing a dried preparation comprising lipids and therapeutic agents, said method comprising the steps of: (a) providing a lipid vesicle particle preparation comprising lipid vesicle particles and at least one solubilized first therapeutic agent; (b) sizing the lipid vesicle particle preparation to form a sized lipid vesicle particle preparation comprising sized lipid vesicle particles and said at least one solubilized first therapeutic agent, said sized lipid vesicle particles having a mean particle size of ≤120 nm and a polydispersity index (PDI) of ≤0.1; (c) mixing the sized lipid vesicle particle preparation with at least one second therapeutic agent to form a mixture, wherein said at least one second therapeutic agent is solubilized in the mixture and is different from said at least one solubilized first therapeutic agent; and (d) drying the mixture formed in step (c) to form a dried preparation comprising lipids and therapeutic agents.

(2) The method of paragraph (1), wherein prior to step (b) the lipid vesicle particles are not sized. For example, and without limitation, prior to step (b) the lipid vesicle particles have not undergone, nor have they been subjected to, any processing step(s) that results in a sizing of the lipid vesicle particles. In an embodiment, the lipid vesicle particles of the lipid vesicle particle preparation of step (a) are of any size and of any distribution of size. In an embodiment, the lipid vesicle particles of the lipid vesicle particle preparation of step (a) are of a size and size distribution as would naturally result by preparing the lipid vesicle particles as described herein.

(3) The method of paragraph (1) or (2), wherein, in step (a), the lipid vesicle particles and the at least one solubilized first therapeutic agent are in sodium acetate sodium phosphate.

(4) The method of any one of paragraphs (1) to (3), wherein, in step (a), the lipid vesicle particles and the at least one solubilized first therapeutic agent are in 25-250 mM sodium acetate having a pH in the range of 6.0-10.5 or 25-250 mM sodium phosphate having a pH in the range of 6.0-8.0.

(5) The method of any one of paragraphs (1) to (4), wherein, in step (a), the lipid vesicle particles and the at least one solubilized first therapeutic agent are in 50 mM sodium acetate having a pH of 6.0±1.0, 100 mM sodium acetate having a pH of 9.5±1.0, 50 mM sodium phosphate having a pH of 7.0±1.0 or 100 mM sodium phosphate having a pH of 6.0±1.0.

(6) The method of any one of paragraphs (1) to (5), wherein, in step (a), the lipid vesicle particles and the at least one solubilized first therapeutic agent are in 100 mM sodium acetate having a pH of 9.5±0.5.

(7) The method of any one of paragraphs (1) to (6), wherein, in step (a), the lipid vesicle particle preparation further comprises a solubilized adjuvant.

(8) The method of any one of paragraphs (1) to (6), wherein step (a) comprises: (a1) providing a therapeutic agent stock comprising the at least one solubilized first therapeutic agent, and optionally further comprising a solubilized adjuvant; and (a2) mixing the therapeutic agent stock with a lipid mixture to form the lipid vesicle preparation.

(9) The method of paragraph (7) or (8), wherein the solubilized adjuvant is encapsulated in the lipid vesicle particles.

(10) The method of any one of paragraphs (7) to (9), wherein the adjuvant is a polyI:C polynucleotide adjuvant.

(11) The method of any one of paragraphs (1) to (10), wherein, in step (a), the at least one solubilized first therapeutic agent is encapsulated in the lipid vesicle particles.

(12) The method of any one of paragraphs (1) to (11), wherein each of the first and second therapeutic agents is independently selected from the group consisting of a peptide antigen, a DNA or RNA polynucleotide that encodes a polypeptide, a hormone, a cytokine, an allergen, a catalytic DNA (deoxyribozyme), a catalytic RNA (ribozyme), an antisense RNA, an interfering RNA, an antagomir, a small molecule drug, a biologic drug, an antibody, or a fragment or derivative of any one thereof; or a mixture thereof.

(13) The method of any one of paragraphs (1) to (12), wherein each of the first and second therapeutic agents is a peptide antigen.

(14) The method of any one of paragraphs (1) to (13), wherein, in step (a), one, two, three, four or five different solubilized first therapeutic agents are in the lipid vesicle particle preparation.

(15) The method of any one of paragraphs (1) to (14), wherein, in step (a), four different solubilized first therapeutic agents are in the lipid vesicle particle preparation.

(16) The method of paragraph (15), wherein the four different solubilized first therapeutic agents are peptide antigens, wherein the first peptide antigen comprises the amino acid sequence FTELTLGEF (SEQ ID NO: 1); the second peptide antigen comprises the amino acid sequence LMLGEFLKL (SEQ ID NO: 2); the third peptide antigen comprises the amino acid sequence STFKNWPFL (SEQ ID NO: 3); and the fourth peptide antigen comprises the amino acid sequence LPPAWQPFL (SEQ ID NO: 4).

(17) The method of any one of paragraphs (1) to (16), wherein, in step (c), the sized lipid vesicle particle preparation is mixed with one, two, three, four or five different second therapeutic agents.

(18) The method of any one of paragraphs (1) to (17), wherein, in step (c), the sized lipid vesicle particle preparation is mixed with one second therapeutic agent.

(19) The method of paragraph (18), wherein the one second therapeutic agent is a peptide antigen comprising the amino acid sequence RISTFKNWPK (SEQ ID NO: 6).

(20) The method of any one of paragraphs (1) to (19), wherein, in step (b), the lipid vesicle particle preparation of step (a) is sized by high pressure homogenization, sonication or membrane extrusion.

(21) The method of paragraph (20), wherein, in step (b), the lipid vesicle particle preparation of step (a) is sized by extrusion through a 0.2 μm polycarbonate membrane followed by extrusion through a 0.1 μm polycarbonate membrane.

(22) The method of paragraph (21), wherein the lipid vesicle particle preparation is sized by extrusion through the 0.2 μm polycarbonate membrane 20 to 40 times and extrusion through the 0.1 μm polycarbonate membrane 10 to 20 times.

(23) The method of any one of paragraphs (20) to (22), wherein the membrane extrusion is performed at 1000 to 5000 psi back pressure.

(24) The method of any one of paragraphs (20) to (23), wherein the at least one solubilized first therapeutic agent is soluble at alkaline pH during high pressure membrane extrusion at about 5000 psi.

(25) The method of any one of paragraphs (1) to (24), wherein the at least one second therapeutic agent is solubilized in mild acetic acid prior to mixing with the sized lipid vesicle particle preparation in step (c).

(26) The method of any one of paragraphs (1) to (25), wherein step (c) further comprises mixing, in any order, at least one T-helper epitope with the sized lipid vesicle particle preparation and the at least one second therapeutic agent, wherein the at least one T-helper epitope is solubilized in the mixture.

(27) The method of paragraph (26), wherein the T-helper epitope comprises the amino acid sequence AQYIKANSKFIGITEL (SEQ ID NO: 5).

(28) The method of paragraph (26) or (27), wherein step (c) comprises: (c1) providing a one or more therapeutic agent stocks comprising a solubilized second therapeutic agent, and a stock comprising the T-helper epitope; and (c2) mixing the stocks with the sized lipid vesicle particles to form the mixture.

(29) The method of paragraph (28), wherein the one or more therapeutic agent stocks are prepared in mild acetic acid.

(30) The method of any one of paragraphs (1) to (29), wherein the mean particle size of the sized lipid vesicle particles is between about 80 nm and about 120 nm.

(31) The method of any one of paragraphs (1) to (30), wherein the mean particle size of the sized lipid vesicle particles is about 80 nm, about 81 nm, about 82 nm, about 83 nm, about 84 nm, about 85 nm, about 86 nm, about 87 nm, about 88 nm, about 89 nm, about 90 nm, about 91 nm, about 92 nm, about 93 nm, about 94 nm, about 95 nm, about 96 nm, about 97 nm, about 98 nm, about 99 nm, about 100 nm, about 101 nm, about 102 nm, about 103 nm, about 104 nm, about 105 nm, about 106 nm, about 107 nm, about 108 nm, about 109 nm, about 110 nm, about 111 nm, about 112 nm, about 113 nm, about 114 nm or about 115 nm.

(32) The method of any one of paragraphs (1) to (31), wherein the mean particle size of the sized lipid vesicle particles is ≤100 nm.

(33) The method of any one of paragraphs (1) to (32), wherein the lipid vesicle particles comprise a synthetic lipid.

(34) The method of paragraph (33), wherein the lipid vesicle particles comprise synthetic dioleoyl phosphatidylcholine (DOPC) or synthetic DOPC and cholesterol.

(35) The method of paragraph (34), wherein the lipid vesicle particles comprise synthetic DOPC and cholesterol at a DOPC:cholesterol ratio of 10:1 (w/w).

(36) The method of any one of paragraphs (1) to (35), wherein the lipid vesicle particles are liposomes.

(37) The method of paragraph (36), wherein the liposomes are unilamellar, multilamellar, or a mixture thereof.

(38) The method of any one of paragraphs (1) to (37) further comprising a step of sterile filtration of the mixture formed in step (c) prior to drying.

(39) The method of any one of paragraphs (1) to (38) further comprising, between steps (c) and (d), a step of confirming that the sized lipid vesicle particles still have a mean particle size of ≤120 nm and a polydispersity index (PDI) of ≤0.1.

(40) The method of any one of paragraphs (1) to (39), wherein the drying is performed by lyophilization, spray freeze-drying, or spray drying.

(41) The method of paragraph (40), wherein the drying is performed by lyophilization.

(42) The method of paragraph (41), wherein the lyophilization is performed by loading one or more containers comprising the mixture of step (c) into a bag, sealing the bag to form a sealed unit, and lyophilizing the sealed unit in a freeze-dryer.

(43) The method of paragraph (42), wherein the bag is a sterile, autoclaved bag.

(44) The method of paragraph (42) or (43), wherein the freeze-dryer is a benchtop freeze dryer.

(45) The method of any one of paragraphs (42) to (44), wherein the freeze-dryer contains more than one sealed unit during the lyophilization.

(46) The method of paragraph (45), wherein each sealed unit contains a different mixture prepared by steps (a) to (c).

(47) A method for preparing a pharmaceutical composition comprising solubilizing the dried preparation obtained by the method of any one of paragraphs (1) to (46) in a hydrophobic carrier.

(48) The method of paragraph (47), wherein the hydrophobic carrier is mineral oil or a mannide oleate in mineral oil solution.

(49) The method of paragraph (47) or (48), wherein the hydrophobic carrier is Montanide® ISA 51.

(50) A pharmaceutical composition prepared by the method of any one of paragraphs (47) to (49).

(51) The pharmaceutical composition of paragraph (50), wherein the lipids are in the form of one or more lipid-based structures having a single layer lipid assembly in the hydrophobic carrier.

(52) The pharmaceutical composition of paragraph (51), wherein, in the hydrophobic carrier, the lipids are in the form of reverse micelles and/or aggregates of lipids with the hydrophobic part of the lipids oriented outwards toward the hydrophobic carrier and the hydrophilic part of the lipids aggregating as a core.

(53) The pharmaceutical composition of paragraph (51) or (52), wherein the size of the lipid-based structures is between about 5 nm to about 10 nm in diameter.

(54) A stable, water-free pharmaceutical composition comprising one or more lipid-based structures having a single layer lipid assembly, at least two different therapeutic agents, and a hydrophobic carrier.

(55) The pharmaceutical composition of paragraph (54), wherein the therapeutic agents are independently selected from the group consisting of a peptide antigen, a DNA or RNA polynucleotide that encodes a polypeptide, a hormone, a cytokine, an allergen, a catalytic DNA (deoxyribozyme), a catalytic RNA (ribozyme), an antisense RNA, an interfering RNA, an antagomir, a small molecule drug, a biologic drug, an antibody, or a fragment or derivative of any one thereof; or a mixture thereof.

(56) The pharmaceutical composition of paragraph (54) or (55), wherein the therapeutic agents are peptide antigens.

(57) The pharmaceutical composition of paragraph (56), which comprises two, three, four, five or more different peptide antigens.

(58) The pharmaceutical composition of paragraph (57), which comprises five different peptide antigens.

(59) The pharmaceutical composition of paragraph (57), wherein the first peptide antigen comprises the amino acid sequence FTELTLGEF (SEQ ID NO: 1); the second peptide antigen comprises the amino acid sequence LMLGEFLKL (SEQ ID NO: 2); the third peptide antigen comprises the amino acid sequence STFKNWPFL (SEQ ID NO: 3); the fourth peptide antigen comprises the amino acid sequence LPPAWQPFL (SEQ ID NO: 4); and the fifth peptide antigen comprising the amino acid sequence RISTFKNWPK (SEQ ID NO: 6).

(60) The pharmaceutical composition of any one of paragraphs (56) to (59), wherein each of the peptide antigens is, independently, at a concentration of between about 0.1 μg/μl and about 5.0 μg/μl.

(61) The pharmaceutical composition of any one of paragraphs (56) to (60), wherein each of the peptide antigens is, independently, at a concentration of about 0.25 μg/μl, about 0.5 μg/μl, about 0.75 μg/μl, about 1.0 μg/μl, about 1.25 μg/μl, about 1.5 μg/μl, about 1.75 μg/μl, about 2.0 μg/μl, about 2.25 μg/μl or about 2.5 μg/μl.

(62) The pharmaceutical composition of any one of paragraphs (56) to (60), which comprises five different peptide antigens, each at a concentration of at least about 1.0 μg/μl.

(63) The pharmaceutical composition of any one of paragraphs (54) to (62), further comprising one or both of a T-helper epitope and an adjuvant.

(64) The pharmaceutical composition of paragraph (63), wherein the T-helper epitope comprises the amino acid sequence AQYIKANSKFIGITEL (SEQ ID NO: 5) and the adjuvant is a polyI:C polynucleotide adjuvant.

(65) The pharmaceutical composition of any one of paragraphs (54) to (64), wherein the hydrophobic carrier is mineral oil or a mannide oleate in mineral oil solution.

(66) The pharmaceutical composition of any one of paragraphs (54) to (65), wherein the hydrophobic carrier is Montanide® ISA 51.

(67) The pharmaceutical composition of any one of paragraphs (54) to (66), wherein the one or more lipid-based structures having a single layer lipid assembly comprise aggregates of lipids with the hydrophobic part of the lipids oriented outwards toward the hydrophobic carrier and the hydrophilic part of the lipids aggregating as a core.

(68) The pharmaceutical composition of any one of paragraphs (54) to (67), wherein the one or more lipid-based structures having a single layer lipid assembly comprise reverse micelles.

(69) The pharmaceutical composition of any one of paragraphs (54) to (68), wherein the size of the lipid-based structures is between about 5 nm to about 10 nm in diameter.

(70) The pharmaceutical composition of any one of paragraphs (54) to (69), wherein one or more of the therapeutic agents are inside the lipid-based structures.

(71) The pharmaceutical composition of any one of paragraphs (54) to (70), wherein one or more of the therapeutic agents are outside the lipid-based structures.

(72) The pharmaceutical composition of any one of paragraphs (54) to (71), which is a clear solution.

(73) The pharmaceutical composition of any one of paragraphs (54) to (72), which has no visible precipitate.

(74) A method of inducing an antibody and/or CTL immune response in a subject comprising administering to the subject the pharmaceutical composition of any one of paragraphs (51) to (73).

(75) The method of paragraph (74), which is for treating cancer or an infectious disease.

(76) Use of the pharmaceutical composition of any one of paragraphs (51) to (73) for inducing an antibody and/or CTL immune response in a subject.

(77) The use of paragraph (76), which is for the treatment of cancer or an infectious disease.

(78) A kit for preparing a pharmaceutical composition for inducing an antibody and/or CTL immune response, the kit comprising: a container comprising a dried preparation prepared by the method of any one of paragraphs (1) to (46); and a container comprising a hydrophobic carrier.

(79) The kit of paragraph (78), wherein the dried preparation comprises five or more different peptide antigens.

(80) The kit of paragraph (78) or (79), wherein the hydrophobic carrier is mineral oil or a mannide oleate in mineral oil solution.

The invention is further illustrated by the following non-limiting examples.

### EXAMPLES

The invention will now be described by way of non-limiting examples having regard to the appended drawings.

### Experimental Protocols

This section describes experimental protocols and techniques that were used in the examples herein. The protocols and techniques are exemplary and the skilled person will understand alternate methods that may be used and/or modifications that may be made to the protocols and techniques to achieve the desired result.

As used in this section, the reference to “Ph.Eur.” is to the European Pharmacopoeia, 9^{th} edition. As used in this section, the reference to “USP” is to the United States Pharmacopeia.

Peptide Assay by RP-HPLC

Identification and quantification of peptides was performed using a reversed-phase HPLC (RP-HPLC) method. The method utilizes an Agilent 1100 Series HPLC system equipped with a Phenomenex Luna 5 μm C8(2) column. The mobile phase is a gradient of 16-37% (v/v) acetonitrile in 0.1% (v/v) aqueous trifluoroacetic acid. Column temperature is maintained at 50° C., and UV-PDA detection is performed at 215 nm. The assay may also be used to identify peptide impurities. The tests were validated to the extent required for a phase 1/2 clinical phase study (data not included).

Polynucleotide Assay by Ion-Exchange HPLC

Identification and quantification of polynucleotides was performed using an anion-exchange HPLC (IEX-HPLC) method. The method utilizes an Agilent 1100 Series HPLC system equipped with a Waters Gen-Pak FAX column. The mobile phase is a gradient of 50-450 mM sodium chloride in 15% (v/v) acetonitrile/100 mM TRIS at pH 8.0. The column temperature is maintained at 25° C., and UV-PDA detection is performed at 260 nm. The tests were validated to the extent required for a phase 1/2 clinical phase study (data not included).

Lipid Assay and Degradant Limit Test by RP-HPLC

Identification and quantification of lipids (e.g. DOPC and cholesterol), and limit testing of the major degradants (e.g. LPC, oleic acid, 70-hydroxycholesterol and 7-ketocholesterol) was performed using a reversed-phase HPLC (RP-HPLC) method. The method utilizes an Agilent 1100 Series HPLC system equipped with a Phenomenex Gemini-NX 3 μm C18 column. The mobile phase is 93% (v/v) methanol in 0.1% (v/v) aqueous trifluoroacetic acid. Column temperature is maintained at 60° C. and UV-PDA detection is performed at 205 nm. The assay may also be used to identify lipid impurities (e.g. DOPC and cholesterol impurity). The tests were validated to the extent required for a phase 1/2 clinical phase study (data not included).

Particle Size Test by DLS

Particle size analysis was performed using a dynamic light scattering (DLS) instrument (Malvern Zetasizer Nano S) for in-process samples at Albany Molecular Research Inc. Burlington (AMRI; Burlington, Mass., USA). In alternate methods, particle size was determined by small angle X-ray scattering (SAXS) as described herein.

Viscosity

Viscosity was carried out in accordance with Ph.Eur. 2.2.9, Capillary Viscometer Method.

pH Testing

The determination of pH was performed according to Ph.Eur. 2.2.3 as well as USP<791>.

Appearance of Reconstituted Solution

The appearance of the compositions was visually inspected according to Ph.Eur. 2.9.20.

Subvisible Particle Testing

A microscopic particle count test was performed on the compositions as per the current version of USP <788>, Method 2. Each of 10 sample vials of dried preparation was solubilized with 0.7 mL of oil and pooled into 100 mL of particle-free ethanol prior to filtration.

Immunogenicity

Immunogenicity was assessed using a DC-ELISpot method. Briefly, HLA-A2 transgenic mice were immunized with 50 μL of the respective composition. Eight days later, the mice were euthanized and lymph node cells were harvested and stimulated in vitro by peptide-loaded target cells and unloaded target cells (for background response) on an ELISpot plate. Antigen-specific release of interferon-γ (IFN-γ) was quantified on the ELISpot plate as a measure of immunogenicity.

Results are recorded as a Pass or Fail based on criteria similar to that used in clinical trials. The composition passes the test if the average antigen-specific response in responding HLA-A2 transgenic mice is at least 10 SFU higher than the background response and the difference is statistically different, calculated using two-tailed paired Students' t test. A minimum of 5 mice are used to test the compositions (individual mouse samples run in duplicates). Due to the potential for mice not-responding to the composition, the test is only considered valid if more than 3 mice respond to the vaccine.

Sterility and Endotoxin

The sterility and endotoxin testing were performed according to current USP methods (USP<71> and USP<85>, respectively). The tests were appropriately validated in accordance with Ph.Eur. 2.6.1 and USP<71> (sterility validation) and Ph.Eur. 2.6.14 and USP<85>(bacterial endotoxin validation).

Content Uniformity

The tests on content uniformity were carried out in accordance with Ph. Eur. 2.9.6, Test A. Conditions for the RP-HPLC method are given below.

TABLE 1
Conditions for RP-HPLC for content uniformity

| Parameter | Description |
|---|---|
| Apparatus | Agilent 1100 Series HPLC system or any system |
| | suitable for gradient reversed phase HPLC |
| Detector | PDA (DAD) detector, 215 nm |
| Column | Phenomenex Luna 5 μm, C8(2), 150 × |
| | 4.6 mm, 100 Å or equivalent |
| Column Temperature | 50° C. |
| Mobile Phase | Mobile phase A: water, acetonitrile and TFA |
| | (900:100:1 v/v) |
| | Mobile phase B: water, acetonitrile and TFA |
| | (600:400:1 v/v) |

Extractable Volume

The tests for extractable volume of the compositions from a syringe was performed in accordance with Ph.Eur 2.9.17.

Moisture Content

Moisture content analysis was performed at AMRI using a coulometric Karl Fischer titration apparatus (Hiranuma AquaCounter AQ-300), qualified as an in-house method based on USP<921>Ic. The dried preparations were dissolved in anhydrous methanol and analyzed as a liquid.

### Example 1

Preparation of Solutions

The following solutions were mixed under laminar flow into a sterile container in a Grade C clean room to minimize bioburden.

0.25% (w/w) Acetic Acid Reagent Solution: Precisely weigh 7.50±0.07 g of glacial acetic acid and dilute in 2970.0±2.9 g of sterile water. Mix thoroughly on a magnetic stir plate (Speed: 200±20 rpm, 5 minutes). Bring the solution to 3000.0±3.0 g with sterile water.

0.2 M Sodium Hydroxide Reagent Solution: Precisely weigh 6.00±0.06 g of sodium hydroxide pellets and dissolve it with stirring in 600.0±0.6 g of sterile water. Mix solution thoroughly on a magnetic stir plate (Speed: 200±20 rpm, 5 minutes). Bring the solution to 750.0±0.75 g with sterile water.

0.1 M Sodium Acetate Buffer pH 9.5±0.5 Reagent Solution: Precisely weigh 163.3±1.6 g of sodium acetate trihydrate powder in 10180.0±10.2 g of sterile water. Mix thoroughly solution on a magnetic stir plate (Speed: 200±20 rpm, 5 minutes). Adjust pH of the solution to 9.5±0.5 using 0.2 M sodium hydroxide solution or 0.25% acetic acid solution. Bring the solution to 12000.0±120.0 g with sterile water.

Dried Preparation Comprising Lipids and a Therapeutic Agent

Formulated with Sized Lipid Vesicle Particles

To prepare a dried preparation comprising lipids and therapeutic agent using sized lipid vesicle particles, the following stock solutions were prepared:

| Stock # | Component | Solvent |
|---|---|---|
| 1 | DNA based PolyI:C | Water |
| | polynucleotide | |
| | adjuvant (dIdC) (0.4 mg/mL) | |
| 2 | SurA24 Peptide Antigen | 0.1 M Sodium |
| | (1 mg/mL): | Acetate |
| | STFKNWPFL (SEQ ID NO: 3) | |
| 3 | SurB7 Peptide Antigen | 0.1 M Sodium |
| | (1 mg/mL): | Acetate |
| | LPPAWQPFL (SEQ ID NO: 4) | |
| 4 | SurA1.T Peptide Antigen | 0.1 M Sodium |
| | (1 mg/mL): | Hydroxide |
| | FTELTLGEF (SEQ ID NO: 1) | |
| 5 | SurA2.M Peptide Antigen | 0.1 M Sodium |
| | (1 mg/mL): | Hydroxide |
| | LMLGEFLKL (SEQ ID NO: 2) | |

The peptides were prepared by PolyPeptide Laboratories (San Diego, Calif., USA) or Girindus AG (Torrance, Calif., USA) as high purity GMP grade starting materials. The polynucleotide adjuvant is fully synthetic and was produced as research grade and GMP grade by BioSpring GmbH (Frankfurt, Germany).

The stock solutions were added to sodium acetate buffer (0.1M, pH 9.5) in the following order: (4), (2), (3), (5) and then (1). The pH was adjusted to 10.0±0.5.

A 10:1 (w:w) homogenous lipid-mixture of DOPC and cholesterol (Lipoid GmbH, Germany) was weighed to obtain 132 g/mL of the lipid-mixture and added to the peptide/polynucleotide solution to form an intermediate bulk (non-sized) and mixed using a Silverson high speed mixer. The pH was adjusted to 10.0±0.5, if required. The intermediate bulk was then sized using an Emulsiflex C55 extruder by passing the material 35 times through a 0.2 μm polycarbonate membrane and then 10 times through a 0.1 μm polycarbonate membrane to attain a particle size of ≤120 nm with a pdi of ≤0.1. The pH was checked every hour during the extrusion and adjusted to 10.0±0.5, if required. Before proceeding to the next step, sizing was confirmed to be 116.3 nm with a PDI of 0.1 by DLS particle size analysis in a Malvern DLS ZETASIZER NANO-S particle size analyzer.

Further stock solutions were prepared as follows:

| Stock # | Component | Solvent |
|---|---|---|
| 6 | SurA3.K Peptide Antigen | 0.25% (w/w) |
| | (1 mg/mL): RISTFKNWPK | Acetic Acid |
| | (SEQ ID NO: 6) | |
| 7 | A16L T-helper epitope | 0.25% (w/w) |
| | (0.5 mg/mL): | Acetic Acid |
| | AQYIKANSKFIGITEL | |
| | (SEQ ID NO: 5) | |

The peptide antigen and A16L T-helper epitope were prepared by PolyPeptide Laboratories (San Diego, Calif., USA) or Girindus AG (Torrance, Calif., USA) as high purity GMP grade starting materials. The RISTFKNWPK (SEQ ID NO: 6) peptide antigen and A16L T-helper epitope were added later in the process due to precipitation issues that occur if the peptide is added before sizing.

Stock solutions (6) and (7) were added to the sized lipid vesicle particle bulk immediately after preparation. The final pH of the solution is adjusted to 7.0±0.5. The final preparation was then sterile filtered using redundant filtration lines, consisting of two 0.22 μm Millipore Millipak 200 filters. The filtration, using nitrogen for positive displacement pressure (20-50 psi), was performed for approximately 15 minutes.

After sterile filtration, the final bulk was filled aseptically into vials and freeze-dried. The freeze-drying was performed according to the following exemplary protocol:

TABLE 2
Equipment & Specifications

| | | |
|---|---|---|
| Fill volume | 1.6 | mL |
| Vial capacity | 3 | mL |

| | |
|---|---|
| Equipment | Vertis bench top freeze dryer or |
| | Lyomax large scale freeze dryers |
| Cycle duration | About 65-75 hrs |
| Residual moisture | <5% |

Vial Specifications:
Description: Vial 2ML 13MM FTN BB LYO PF
Supplier: West Pharmaceuticals

Stopper Specifications:
Description: Fluorotec Lyophilization Closure, 13MM (V2 F452W DV LYO
D777-1 NO B2)
Supplier: West Pharmaceuticals

Seal Specifications:
Description: West-Spectra Flip-Off 13 mm Seal
Supplier: West Pharmaceuticals

TABLE 3
Exemplary Lyophilization (freeze-dry) Protocol

| | | | | | | | | Cond. |
|---|---|---|---|---|---|---|---|---|
| | | | | | | | | Temp |
| | | Shelf Temp | Gas | | | | Step | Set |
| | | Set Point | (0 = N2, | Ram | Pressure/ | | Time | Point |
| Step | Step Type | (° C.) | 1 = Air) | Pressure | Vacuum | | (hh:mm) | (° C.) |
| 1 | Loading | 20 | | | | | | |
| 2 | Freezing | 5 | | | | | 00:20 | |
| 3 | Freezing | 5 | | | | | 02:00 | |
| 4 | Freezing | −50 | | | | | 00:55 | |
| 5 | Freezing | −50 | | | | | 02:00 | |
| 6 | Freezing | −7 | | | | | 01:43 | |
| 7 | Freezing | −7 | | | | | 03:00 | |
| 8 | Freezing | −50 | | | | | 00:45 | |
| 9 | Freezing | −50 | | | | | 02:00 | |
| 10 | Evacuation | −50 | | | 100 | micron | | −75 |
| 11 | Drying | −50 | | | 100 | micron | 0:30 | −75 |
| 12 | Drying | −40 | | | 100 | micron | 5:20 | −75 |
| 13 | Drying | −40 | | | 100 | micron | 55:00 | −75 |
| 14 | Drying | 35 | | | 100 | micron | 23:00 | −75 |
| 15 | Drying | 35 | | | 100 | micron | 2:00 | −75 |
| 16 | Drying | 25 | | | 100 | micron | 0:10 | −75 |
| 17 | Drying | 25 | | | 100 | micron | 1:00 | −75 |
| 18 | Aeration | | 0 | | 0.9 | Psia | 0 sec | |

Throughout the process, the peptide content was analyzed. The lyophilisates were stoppered, capped, and a 100% visualisation check was performed.

This dried preparation is hereinafter referred to as Batch #1.

Formulated without Sizing the Lipid Vesicle Particles

To prepare a dried preparation comprising lipids and therapeutic agent without sizing of the lipid vesicle particles, the procedure above was followed with the exception that the intermediate bulk was not sized prior to addition of stock solutions (6) and (7).

This dried preparation is hereinafter referred to as Batch #2.

Formulated without Lipids

To prepare a dried preparation comprising therapeutic agent without lipids, the procedure above was followed with the exception that the lipid-mixture was not added to the peptide/polynucleotide solution and the peptide/polynucleotide solution was not sized prior to addition of stock solutions (6) and (7). In essence, the stock solutions were added to sodium acetate buffer (0.1M, pH 9.5) in the following order: (4), (2), (3), (5), (1), (6) and then (7). The pH was adjusted to 7.0±0.5, followed by sterile filtration and freeze-drying.

This dried preparation is hereinafter referred to as Batch #3.

Pharmaceutical Composition

The dried preparations of Batches #1, #2 and #3 were each separately solubilized in an oil diluent (i.e. Montanide® ISA 51) to provide final compositions with the profile shown in the table below:

TABLE 4
Exemplary Product Profile

| | Concentration in Final |
|---|---|
| | Composition |
| Component | (mg/mL) |
| Peptide Antigens | 1.00 |
| Adjuvant | 0.40 |
| T-helper epitope | 0.50 |
| DOPC/cholesterol (10:1 w/w) | 132.00 |
| [Batch #1 and 2 only] | |

The characteristics of the resultant compositions after solubilization are described in the table below and in FIG. 1.

TABLE 5
Product Characteristics

| Batch | Formulation Method | Product Description |
|---|---|---|
| Composition | With sizing of lipid | Clear solution, practically |
| from Batch #1 | vesicle particles | free of particles |
| | | [FIG. 1A] |
| Composition | Without lipids | Dense hazy and turbid solution |
| from Batch #3 | | [FIG. 1B] |
| Composition | Without sizing of lipid | Dense hazy and turbid solution |
| from Batch #2 | vesicle particles | [FIG. 1C] |

After solubilization in the hydrophobic carrier, a clear, pale yellow solution practically free of particles is obtained when the dried lipid/therapeutic agent preparation is prepared by sizing the lipid vesicle particles to a mean particle size of ≤120 nm and a PDI≤0.1 (FIG. 1A). Notably, the optically clear solution has a similar appearance as Montanide ISA 51 VG alone. In contrast, simple mixing of the peptides and polynucleotide adjuvant with the hydrophobic carrier gives a turbid suspension (FIG. 1B). Likewise, a composition prepared with non-sized lipid particles also gives a turbid suspension (FIG. 1C).

Thus, sizing of the lipid particles to a mean particle size of ≤120 nm and a PDI≤0.1 is required to produce a suitable dried preparation that disintegrates easily once the hydrophobic carrier is added.

### Example 2

The percent solubilization of the peptides in each the compositions prepared from the dried preparations of Batches #1, #2 and #3 was evaluated by centrifuging samples of the compositions at 10,000 rpm and analyzing the peptide content in the supernatant by RP-HPLC.

The percent solubilization in the composition prepared using sized lipid vesicle particles was found to be >98% for the peptide antigens and 84% for the A16L T-helper epitope. In contrast, the percent solubilization of the peptide antigens and A16L T-helper epitope in the non-lipid formulation was practically zero and that in the non-sized lipid formulation was significantly reduced.

TABLE 6
Percent Solubilization of peptides

| | % Solubilization | | | | | |
|---|---|---|---|---|---|---|
| | SurA3.K Peptide | A16L | SurA1.T Peptide | SurA24 Peptide | SurB7 Peptide | SurA2.M Peptide |
| | RISTFKNWPK | T-helper | FTELTLGEF | STFKNWPFL | LPPAWQPFL | LMLGEFTLKL |
| Sample | (SEQ ID NO: 6) | Epitope | (SEQ ID NO: 1) | (SEQ ID NO: 3) | (SEQ ID NO: 4) | (SEQ ID NO: 2) |
| Composition | 98.58% | 84.28% | 98.15% | 99.47% | 98.87% | 100.05% |
| prepared | | | | | | |
| with sized | | | | | | |
| lipid vesicle | | | | | | |
| particles | | | | | | |
| Composition | 0.00% | 0.00% | 0.00% | 0.79% | 1.56% | 0.00% |
| prepared | | | | | | |
| with no lipids | | | | | | |
| Composition | 25.50% | 31.01% | 16.26% | 35.91% | 28.34% | 19.12% |
| prepared | | | | | | |
| with non- | | | | | | |
| sized lipid | | | | | | |
| vesicle | | | | | | |
| particles | | | | | | |

It was unexpectedly found that even when certain peptides (SurA3.K and A16L T-helper epitope) are not actively incorporated into the lipid vesicle particles (e.g. encapsulated), but rather are added outside the sized lipid vesicle particles, it was still possible to solubilize these peptides to the same degree in a hydrophobic carrier as peptides that were added earlier in the process when the lipid vesicle particles were being formed.

This is an advantageous property since it was found during process development that when SurA3.K and A16L peptide were combined with the other four survivin peptide antigens, polynucleotide adjuvant and lipid mixture, an aggregation and/or precipitation of SurA3.K and A16L occurred. This aggregation and/or precipitation was accelerated by frequent agitation of the intermediate bulk product. Moreover, the high flow rate during the size extrusion of the intermediate bulk, wherein the solution was continuously extruded at 50 L/hr through 0.22 μm and 0.10 μm polycarbonate membranes for about 30 to 50 passes, accelerated the aggregation of both SurA3.K and A16L peptides, which then accumulated on the polycarbonate membrane. The retention of both SurA3.K and A16L aggregates by the extruder membrane resulted in a significant drop in the concentration of SurA3.K and A16L in the final bulk (approximately 25% loss of SurA3.K and 50% loss of A16L in content from the nominal target limit).

### Example 3

The Batch #1 composition from Example 1 was analyzed by small angle x-ray scattering technique (SAXS) to determine the size and shape of the lipid-based structures present in the hydrophobic carrier when the compositions were prepared with sized lipid vesicle particles.

The SAXS patterns were collected at University of Sherbrooke, QC, Canada, with a Bruker AXS Nanostar system, equipped with a Microfocus Copper Anode at 45 kV/0.65 mA, MONTAL OPTICS and a VANTEC 2000 2D detector at 27.3 cm distances from the samples. The distances were calibrated with a Silver Behenate standard prior to the measurements. The samples were injected into 0.6 mm diameter special glass capillaries, sealed, and placed at predetermined positions. The positioning fine tuning was done by nanography; a 2 second per step scan sweep on X and Y to find the exact position of the samples. The scattering intensities were treated with Primus GNOM 3.0 programs from ATSAS 2.3 software.

Scans were measured for (1) Montanide ISA 51 VG (blank control) and (2) Batch #1 composition. Scans were performed with 800 sec. exposure for the Montanide ISA 51 VG sample and Batch #1 sample. The Montanide ISA 51 VG was mathematically subtracted from the Batch #1 sample to determine the particle size and shape by a pair-distance distribution function. The gaussian curve shape is typical for a spherical particle.

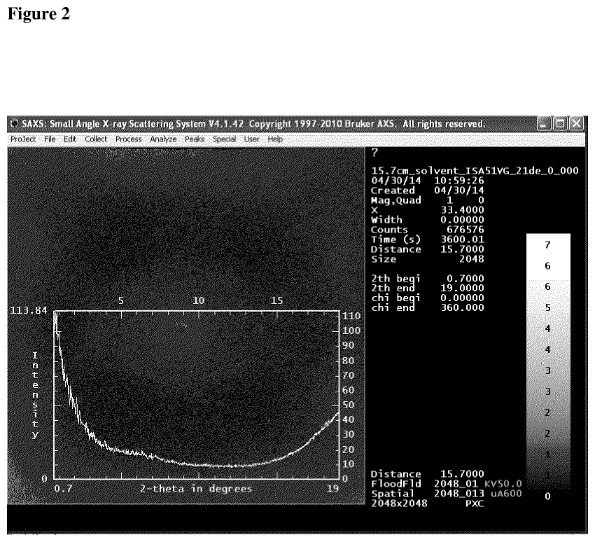

FIG. 2 shows the results for Montanide ISA 51 VG (blank control). No particle structures were observed. As such, no evaluation of particle size was performed.

FIGS. 3 and 4 show the results for the Batch #1 composition. The images indicate that the lipids form a single layer assembly. As shown by the particle size evaluation in FIG. 4, the Dₘₐₓ particle size is about 6.0 nm and the shape estimated by SAXS is spherical. This corresponds to the size of reverse micelles.

SAXS analysis was also performed on a separate composition prepared in accordance with the procedure in Example 1 using sized lipid vesicle particles (Batch #4). The result, together with Batch #1, are shown in the table below:

TABLE 7
SAXS analysis data for compositions prepared
using sized lipid vesicle particles

| | Particle | Particle | Particle | Particle |
|---|---|---|---|---|
| | Size at | Shape at | Size at | Shape at |
| Batch | t = 0 | t = 0 | t = 4 h | t = 4 h |
| Batch #1 Composition | 6.0 nm | spherical | 6.2 nm | spherical |
| Batch #4 Composition | 8.2 nm | spherical | 7.8 nm | spherical |

The data demonstrates that by using sized lipid vesicle particles having a mean particle size of ≤120 nm and a PDI≤0.1, the resultant compositions comprise structures corresponding to reverse micelles, which are spherical in shape with an average diameter of 6 nm to 8 nm. The shape and size did not change after a 4 hour holding time.

### Example 4

The stability of the solubilized composition of Batch #4 (see Example 3), prepared in accordance with Example 1 using sized lipid vesicle particles, was evaluated taking into consideration total impurities, endotoxin levels and the following physical properties: appearance, optical density, viscosity, density, extractable volume, and particle size.

TABLE 8
Stability of solubilized composition of Batch #4 stored in vials at room temperature

| Property of Composition | Specifications | | t = 0 | | t = 4 h | | t = 24 h | |
|---|---|---|---|---|---|---|---|---|
| Visual Appearance of | Clear, free of | | Clear, free of | | Clear, free of | | Clear, free of | |
| Composition | particulates | | particulates | | particulates | | particulates | |
| Optical Density in AU | 0.100 AU ± 0.020 AU | | 0.104 | AU | 0.112 | AU | 0.097 | AU |
| (Absorbance at 450 nm) | | | | | | | | |
| Particle Size (by SAXS) | ≤10 | nM | 8.2 | nm | 7.8 | nm | not done | |
| Viscosity at 23.0 ± 0.5° C. | Report | | 60.1 | cSt | 62.5 | cSt | 63.3 | cSt |
| (cSt) by Ph. Eur. 2.2.9 | | | | | | | | |
| Density at 23.0 ± 0.5° C. | 0.93-0.97 | g/mL | 0.97 | g/mL | 0.97 | g/mL | 0.96 | g/mL |
| Extractable Volume by | Volume ≥ 0.5 mL | | 0.6 | mL | 0.6 | mL | 0.6 | mL |
| Ph. Eur. 2.9.17 | | | | | | | | |
| Endotoxin (Ph. Eur. | <100 | EU/vial | <24 | EU/vial | <24 | EU/vial | not done | |
| 2.6.14, USP<85>) | | | | | | | | |
| Immunogenicity Assay | Average response 10 | | Pass | | Pass | | Pass | |
| (ELISpot) | SFU higher than | | | | | | | |
| | unstimulated controls, | | | | | | | |
| | p < 0.05 | | | | | | | |
| Peptide Content by RP-HPLC | | | | | | | | |
| (% recovery from t = 0) | | | | | | | | |
| SurA3.K | 85-115% of the | | 100.0% | | 96.4% | | 96.1% | |
| A16L | average content at t = 0 | | 100.0% | | 89.8% | | 113.6% | |
| SurA1.T | | | 100.0% | | 100.0% | | 93.5% | |
| SurA24 | | | 100.0% | | 95.9% | | 87.7% | |
| SurB7 | | | 100.0% | | 95.3% | | 88.4% | |
| SurA2.M | | | 100.0% | | 98.0% | | 87.9% | |
| Peptide Impurities by | | | | | | | | |
| RP-HPLC (% Area) | | | | | | | | |
| | Ind. Impurity ≤1% | | Individual 0.27% | | Individual 0.27% | | Individual 0.31% | |
| | Total Impurities ≤5% | | Total 0.27% | | Total 0.27% | | Total 0.31% | |
| Polynucleotide Content by | 85-115% of the | | 100.0% | | 98.6% | | 97.3% | |
| IEX-HPLC | average content at t = 0 | | | | | | | |
| (% recovery from t = 0) | | | | | | | | |
| Lipid Content by | | | | | | | | |
| RP-HPLC | | | | | | | | |
| (% recovery from t = 0) | | | | | | | | |
| DOPC | 85-115% of the | | 100.0% | | 95.3% | | 93.2% | |
| Cholesterol | average content at t = 0 | | 100.0% | | 96.1% | | 93.7% | |
| DOPC degradants by | | | | | | | | |
| RP-HPLC | | | | | | | | |
| LPC | <6 | mg/mL | 1.50 | mg/mL | 1.44 | mg/mL | 1.57 | mg/mL |
| Oleic Acid | <6 | mg/mL | 0.58 | mg/mL | 0.56 | mg/mL | 0.32 | mg/mL |
| Cholesterol degradants | | | | | | | | |
| (RP-HPLC) | | | | | | | | |

| | | | | | |
|---|---|---|---|---|---|
| 7-Hydroxycholesterol | <0.6 | mg/mL | n.d. | n.d. | n.d. |
| 7β-Ketocholesterol | <0.6 | mg/mL | n.d. | n.d. | n.d. |

| | | | | |
|---|---|---|---|---|
| Lipid Impurities | | | | |
| (RP-HPLC) | | | | |
| POPC | ≤3.0% of DOPC area | 2.65% | 1.21% | 1.04% |
| Cholesterol Impurity | ≤1.0% of Chol, area | int | int | int |

n.d. = not detected
int = interference from oil components

After solubilization of the dried lipid/therapeutic agent preparation in Montanide ISA 51 VG, the resultant composition was stable for at least 24 hours at room temperature.

### Example 5

Compatibility with a syringe (e.g. stability within a syringe) was also evaluated at three time points (t=0, 30, and 60 minutes) for the solubilized composition of Batch #4 (see Example 3), prepared in accordance with Example using sized lipid vesicle particles.

After solubilization of the dried lipid/therapeutic agent preparation in Montanide ISA 51 VG, 0.5 mL of the product was drawn into a 1 mL Medallion® syringe (barrel: polycarbonate, plunger: acrylonitrile butadiene, plunger tip: silicone). Stability studies in accordance with the parameters in the table below were evaluated at T=0, T=30 minutes and T=60 minutes.

TABLE 9
Stability of Composition within a Syringe

| Property of Composition | Specifications | | t = 0 | | t = 30 min | | t = 60 min | |
|---|---|---|---|---|---|---|---|---|
| Visual Appearance of | Clear, free of | | Clear, free of | | Clear, free of | | Clear, free of | |
| Composition | particulates | | particulates | | particulates | | particulates | |
| Optical Density in AU | 0.100 AU ± 0.020 AU | | 0.100 | AU | 0.117 | AU | 0.094 | AU |
| (Absorbance at 450 nm) | | | | | | | | |
| Viscosity at 23 ± 0.5° C. | Report | | 59.2 | cSt | 60.5 | cSt | 58.1 | cSt |
| (cSt) by Ph. Eur. 2.2.9 | | | | | | | | |
| Extractable Volume by | Volume ≥ 0.5 mL | | 0.5 | mL | 0.5 | mL | 0.5 | mL |
| Ph. Eur. 2.9.17 | | | | | | | | |
| Endotoxin (Ph. Eur. | <100 | EU/vial | <24 | EU/vial | <24 EU/vial | | not done | |
| 2.6.14, USP<85>) | | | | | | | | |
| Immunogenicity Assay | Average response | | Pass | | Pass | | Pass | |
| (ELISpot) | 10 SFU higher than | | | | | | | |
| | unstimulated controls, | | | | | | | |
| | p < 0.05 | | | | | | | |
| Peptide Content by | | | | | | | | |
| RP-HPLC | | | | | | | | |
| (% recovery from t = 0) | | | | | | | | |
| SurA3.K | 85-115% of the average | | 98.3% | | 96.9% | | 99.8% | |
| A16L | content at t = 0 (in vial) | | 99.3% | | 105.2% | | 105.3% | |
| SurA1.T | | | 100.5% | | 97.8% | | 104.7% | |
| SurA24 | | | 99.7% | | 97.4% | | 101.3% | |
| SurB7 | | | 100.3% | | 98.0% | | 100.7% | |
| SurA2.M | | | 100.4% | | 96.8% | | 103.3% | |
| Peptide Impurities by | | | | | | | | |
| RP-HPLC (% Area) | | | | | | | | |
| | Ind. Impurity ≤1% | | Individual 0.52% | | Individual 0.54% | | Individual 0.26% | |
| | Total Impurities ≤5% | | Total 0.52% | | Total 0.54% | | Total 0.26% | |
| Polynucleotide Content by | 85-115% of the average | | 100.0% | | 93.2% | | 98.7% | |
| IEX-HPLC | content at t = 0 | | | | | | | |
| (% recovery from t = 0) | | | | | | | | |
| Lipid Content by | | | | | | | | |
| RP-HPLC | | | | | | | | |
| (% recovery from t = 0) | | | | | | | | |
| DOPC | 85-115% of the average | | 96.7% | | 98.0% | | 100.3% | |
| Cholesterol | content at t = 0 | | 97.1% | | 97.8% | | 100.6% | |
| DOPC degradants by | | | | | | | | |
| RP-HPLC (mg/mL) | | | | | | | | |
| LPC | <6 | mg/mL | 1.34 | mg/mL | 1.27 | mg/mL | 1.24 | mg/mL |
| Oleic Acid | <6 | mg/mL | 0.62 | mg/mL | 0.61 | mg/mL | 0.60 | mg/mL |
| Cholesterol degradants | | | | | | | | |
| 7-Hydroxycholesterol | <0.6 | mg/mL | n.d. | | n.d. | | n.d. | |
| 7β-Ketocholesterol | <0.6 | mg/mL | n.d. | | n.d. | | n.d. | |
| Lipid Impurities (HPLC) | | | | | | | | |

| | | | | |
|---|---|---|---|---|
| POPC | ≤3.0% of DOPC area | 2.10% | 2.91% | 2.94% |
| Cholesterol Impurity | ≤1.0% of Chol. area | int | int | int |

n.d. = not detected
int = interference from oil components

Adsorption to the device was not observed, as evidenced in the content assays. Additionally, there were no modifications to the peptide antigens in the final composition stored in syringe at room temperature. No significant change was observed in the optical density, viscosity, and extractable volume over the 60 minute period.

### Example 6

Long term stability testing of dried lipid/therapeutic agent preparations prepared in accordance with the procedure above in Example 1 under “Formulated with Sized Lipid Vesicle Particles” have been performed.

Briefly, stability was monitored at −20° C. and 5° C. Stability testing involved analyzing the parameters in the tables below at the given time points.

TABLE 10
Long-term stability data at −20° C. ± 5° C. for compositions prepared using sized lipid vesicle particles

| | | | Months | | | |
|---|---|---|---|---|---|---|
| Parameter | Acceptance Criterion | | 0 | 6 | 12 | 18 |
| Appearance of | Dry, white to off-white, | | Dry, white, | Dry, white, | Dry, white, | Dry, white, |
| lyophilisate | non-collapsed cake | | non-collapsed | non-collapsed | non-collapsed | non-collapsed |
| (visual inspection) | | | cake | cake | cake | cake |
| Solubilization | Report | | 14 minutes | n.d.¹ | 14 minutes | 7 minutes |
| Time | | | | | | |
| Appearance of | Clear, free of | | Clear solution, | n.d.¹ | Clear solution, | Clear solution, |
| Solubilized | particulates | | free of | | free of | free of |
| Product | | | particulates | | particulates | particulates |
| (visual inspection) | | | | | | |
| Particulate Matter | Average number of | | ≥10 μm: 13 | n.d.¹ | n.d.¹ | n.d.¹ |
| (USP 37<788>, | particles present in the | | ≥25 μm: 1 | | | |
| Method 2) | units tested does not | | | | | |
| | exceed 3000 per | | | | | |
| | container ≥10 μm and | | | | | |
| | does not exceed 300 per | | | | | |
| | container ≥25 μm. | | | | | |
| Viscosity @ 23° C. | Report (cSt) | | 49.7 | n.d.¹ | 48.2 | 44.3 |
| (USP<911>) | | | | | | |
| pH Value | 6.5-8.5 | | 7.3 | 7.2 | 7.3 | 7.1 |
| (USP<791>) | | | | | | |
| Peptide Assay | — | | — | — | — | — |
| (RP-HPLC) | | | | | | |
| SurA3.K | 0.80-1.20 | mg/mL | 1.04 | 0.98 | 0.97 | 1.02 |
| A16L | 0.40-0.60 | mg/mL | 0.48 | 0.45 | 0.45 | 0.44 |
| SurA1.T | 0.80-1.20 | mg/mL | 1.03 | 1.04 | 0.99 | 1.04 |
| SurA24 | 0.80-1.20 | mg/mL | 1.00 | 0.99 | 0.97 | 1.00 |
| SurB7 | 0.80-1.20 | mg/mL | 1.09 | 1.01 | 1.01 | 1.03 |
| SurA2.M | 0.80-1.20 | mg/mL | 0.95 | 0.92 | 0.89 | 0.91 |
| Peptide | Individual Impurities: | | Not | Not | Not | Not |
| Impurities/ | ≤1.0 area % | | detected | detected | detected | detected |
| Degradants | Total impurities: ≤5.0 | | Not | Not | Not | Not |
| (RP-HPLC) | area % | | detected | detected | detected | detected |
| Polynucleotide | 0.32-0.48 | mg/mL | 0.43 | 0.42 | 0.43 | 0.42 |
| Adjuvant Assay | | | | | | |
| (IEX-HPLC) | | | | | | |
| Lipid Assay | — | | — | — | — | — |
| (RP-HPLC) | | | | | | |
| DOPC | 96.00-144.00 | mg/mL | 118.41 | 123.60 | 102.63 | 128.00 |
| Cholesterol | 9.60-14.40 | mg/mL | 11.03 | 12.14 | 11.10 | 12.85 |
| Lipid Degradants | — | | — | | | |
| (RP-HPLC) | | | | | | |
| 2-LPC | ≤6.0 | mg/mL | 0.5 | 0.5 | 0.5 | 0.7 |
| Oleic Acid | ≤6.0 | mg/mL | 0.3 | 0.3 | 0.2 | 0.3 |
| 7β- | ≤0.6 | mg/mL | Not | Not | Not | Not |
| Hydroxychoesterol | | | detected | detected | detected | detected |
| 7-ketocholesterol | <0.6 | mg/mL | Not | Not | Not | Not |
| | | | detected | detected | detected | detected |
| Water Content | <5.0% | | 0.2% | 0.2% | 0.1% | 0.3% |
| (USP<921>Ic) | | | | | | |
| Sterility | Must conform | | Negative for | n.d.¹ | n.d.¹ | n.d.¹ |
| (USP<71>) | | | growth | | | |
| Endotoxin | ≤100 | EU/vial | <32 | n.d.¹ | n.d.¹ | n.d.¹ |
| (USP <85>) | | | | | | |

| | | | | | |
|---|---|---|---|---|---|
| Immunogenicity | Average response 10 | Pass | n.d.¹ | Pass | Pass |
| (ELISpot) | SFU higher than | | | | |
| | unstimulated controls, | | | | |
| | p < 0.05 | | | | |
| General Safety | Must conform | Pass | n.d.¹ | n.d.¹ | n.d.¹ |
| Test | | | | | |
| (21 CFR 610.111) | | | | | |

n.d.¹ = not done as per stability testing protocol

TABLE 11
Long-term stability data at 5° C. ± 3° C. for compositions prepared using sized lipid vesicle particles

| | Acceptance | | Months | | | | | |
|---|---|---|---|---|---|---|---|---|
| Parameter | Criterion | | 0 | 3 | 6 | 9 | 12 | 18 |
| Appearance of | Dry, white to | | Dry, white, | Dry, white, | Dry, white, | Dry, white, | Dry, white, | Dry, white, |
| lyophilisate | off-white, | | non- | non- | non- | non- | non- | non- |
| (visual inspection) | non-collapsed | | collapsed | collapsed | collapsed | collapsed | collapsed | cdlapsed |
| | cake | | cake | cake | cake | cake | cake | cake |
| Solubilization Time | Report | | 14 minutes | n.d.¹ | 7 minutes | n.d.¹ | 7 minutes | 7 minutes |
| Appearance of | Clear, free of | | Clear | n.d.¹ | Clear | n.d.¹ | Clear | Clear |
| Solubilized Product | particulates | | solution, | | solution, | | solution, | solution, |
| (visual inspection) | | | free of | | free of | | free of | free of |
| | | | particulates | | particulates | | particulates | particulates |
| Particulate Matter | Average number of | | ≥10 μm: 13 | n.d.¹ | n.d.¹ | n.d.¹ | n.d.¹ | n.d.¹ |
| (USP 37<788>, | particles present | | ≥25 μm: 1 | | | | | |
| Method 2) | in the units tested | | | | | | | |
| | does not exceed | | | | | | | |
| | 3000 per | | | | | | | |
| | container ≥10 μm | | | | | | | |
| | and does not exceed | | | | | | | |
| | 300 per | | | | | | | |
| | container ≥25 μm. | | | | | | | |
| Viscosity @ 23° C. | Report (cSt) | | 49.7 | n.d.¹ | 48.6 | n.d.¹ | 57.9 | 41.6 |
| (USP <911>) | | | | | | | | |
| pH Value | 6.5-8.5 | | 7.3 | 7.3 | 7.2 | 7.5 | 7.5 | 7.2 |
| (USP <791>) | | | | | | | | |
| Peptide Assay | — | | — | — | — | — | — | — |
| (RP-HPLC) | | | | | | | | |
| SurA3.K | 0.80-1.20 | mg/mL | 1.04 | 0.99 | 0.93 | 0.92 | 0.93 | 1.02 |
| A16L | 0.40-0.60 | mg/mL | 0.48 | 0.49 | 0.46 | 0.49 | 0.49 | 0.46 |
| SurA1.T | 0.80-1.20 | mg/mL | 1.03 | 1.02 | 1.03 | 1.02 | 0.97 | 1.07 |
| SurA24 | 0.80-1.20 | mg/mL | 1.00 | 0.98 | 0.98 | 0.95 | 0.95 | 0.99 |
| SurB7 | 0.80-1.20 | mg/mL | 1.09 | 1.01 | 1.00 | 1.00 | 0.99 | 1.03 |
| SurA2.M | 0.80-1.20 | mg/mL | 0.95 | 0.91 | 0.91 | 0.89 | 0.88 | 0.91 |
| Peptide | Individual | | Not | Not | Not | Not | Not | Not |
| Impurities/ | Impurities: ≤1 | | detected | detected | detected | detected | detected | detected |
| Degradants | area % | | | | | | | |
| (RP-HPLC) | Total | | Not | Not | Not | Not | Not | Not |
| | impurities: ≤5 | | detected | detected | detected | detected | detected | detected |
| | area % | | | | | | | |
| Polynucleotide | 0.32-0.48 | mg/mL | 0.43 | 0.41 | 0.42 | 0.44 | 0.42 | 0.41 |
| Adjuvant Assay | | | | | | | | |
| (IEX-HPLC) | | | | | | | | |
| Lipid Assay | — | | — | — | — | — | — | — |
| (RP-HPLC) | | | | | | | | |
| DOPC | 96.00-144.00 | mg/mL | 118.41 | 112.91 | 124.60 | 130.11 | 120.00 | 121.35 |
| Cholesterol | 9.60-14.40 | mg/mL | 11.03 | 11.30 | 12.26 | 12.11 | 12.00 | 12.25 |
| Lipid Degradants | — | | — | — | — | — | — | — |
| (RP-HPLC) | | | | | | | | |
| 2-LPC | ≤6 | mg/mL | 0.5 | 0.5 | 0.5 | 0.6 | 0.5 | 0.6 |
| Oleic Acid | ≤6 | mg/mL | 0.3 | 0.2 | 0.2 | 0.4 | 0.2 | 0.2 |
| 7β- | ≤0.6 | mg/mL | Not | Not | Not | Not | Not | Not |
| Hydroxychoesterol | | | detected | detected | detected | detected | detected | detected |
| 7-ketocholesterol | ≤0.6 | mg/mL | Not | Not | Not | Not | Not | Not |
| | | | detected | detected | detected | detected | detected | detected |
| Water Content | <5.0% | | 0.2% | 0.5% | 0.5% | 0.5% | 0.5% | 0.7% |
| (USP<921>Ic) | | | | | | | | |
| Sterility | Must conform | | Negative | n.d.¹ | n.d.¹ | n.d.¹ | n.d.¹ | n.d.¹ |
| (USP<71>) | | | for growth | | | | | |
| Endotoxin | ≤100 | EU/vial | <32 | n.d.¹ | n.d.¹ | n.d.¹ | n.d.¹ | n.d.¹ |
| (USP<85>) | | | | | | | | |

| | | | | | | | |
|---|---|---|---|---|---|---|---|
| Immunogenicity | Average | Pass | n.d.¹ | Pass | n.d.¹ | Pass | Pass |
| (ELISpot) | response 10 | | | | | | |
| | SFU higher | | | | | | |
| | than | | | | | | |
| | unstimulated | | | | | | |
| | controls, | | | | | | |
| | p < 0.05 | | | | | | |
| General Safety Test | Must conform | Pass | n.d.¹ | n.d.¹ | n.d.¹ | n.d.¹ | n.d.¹ |
| (21 CFR 610.11) | | | | | | | |

n.d.¹ = not done as per stability testing protocol

Stability data collected supports long term stability of the dried lipid/therapeutic agent preparation prepared using sized lipid vesicle particles. The

[symbol]

### Example 7

The reproducibility of the method in Example 1 for preparing a pharmaceutical-grade composition in accordance with Batch #1 using sized lipid vesicle particles and peptide antigen added after extrusion was studied.

Briefly, the procedure above in Example 1 under “Formulated with Sized Lipid Vesicle Particles” was used to prepare dried lipid/therapeutic agent preparations. The dried preparations were solubilized in Montanide® ISA 51 to provide final compositions in accordance with the profile shown in Table 4. Each composition in a separate vial was then evaluated based on the parameters in the table below.

TABLE 12
Reproducibility of physical and chemical properties of compositions prepared using sized lipid vesicle particles

| Property of | Vial | Vial | Vial | Vial | Vial | Average | Standard | % RSD |
|---|---|---|---|---|---|---|---|---|
| Composition | 1 | 2 | 3 | 4 | 5 | (n = 5) | Deviation | (n = 5) |
| Visual Appearance of | Clear | Clear | Clear | Clear | Clear | n/a | n/a | n/a |
| Composition | | | | | | | | |
| Optical Density | 0.097 | 0.107 | 0.092 | 0.111 | 0.111 | 0.104 | 0.009 | 8.35% |
| (Absorbance at 450 nm) | | | | | | | | |
| Viscosity at 23.0 ± 0.5° C. | 58.2 | 61.6 | 60.5 | 60.9 | 59.3 | 60.1 | 1.4 | 2.3% |
| (cSt) by Ph. Eur. 2.2.9 | | | | | | | | |
| Density at 23.0 ± 0.5° C. | 0.94 | 0.94 | 0.97 | 0.97 | 0.96 | 0.96 | 0.02 | 2.0% |
| Extractable Volume | 0.6 | 0.6 | 0.6 | 0.6 | 0.6 | 0.6 | 0.0 | 0.0% |
| (mL) by Ph. Eur. 2.9.17 | | | | | | | | |
| Peptide Content by | | | | | | | | |
| RP-HPLC (mg/mL) | | | | | | | | |
| SurA3.K | 0.86 | 0.90 | 0.91 | 0.88 | 0.92 | 0.89 | 0.02 | 2.6% |
| SurA1.T | 1.07 | 1.10 | 1.09 | 1.07 | 1.11 | 1.09 | 0.02 | 1.7% |
| SurA24 | 0.92 | 0.94 | 0.93 | 0.91 | 0.95 | 0.93 | 0.01 | 1.6% |
| SurB7 | 1.07 | 1.11 | 1.08 | 1.07 | 1.11 | 1.09 | 0.02 | 1.8% |
| SurA2.M | 1.11 | 1.14 | 1.15 | 1.14 | 1.18 | 1.14 | 0.03 | 2.2% |
| Peptide Impurities by | | | | | | | | |
| RP-HPLC (% Area) | | | | | | | | |
| RRT 3.66 LMLGEFLKL | 0.65% | 0.40% | 0.53% | 0.58% | 0.57% | 0.55% | n/a | n/a |
| (M oxidized to sulfoxide; | | | | | | | | |
| SEQ ID NO: 2) | | | | | | | | |
| Polynucleotide Content | 0.36 | 0.37 | 0.38 | 0.38 | 0.36 | 0.37 | 0.01 | 2.7% |
| by IEX-HPLC (mg/mL) | | | | | | | | |
| Lipid Content by | | | | | | | | |
| RP-HPLC (mg/mL) | | | | | | | | |
| DOPC | 117.39 | 121.44 | 120.26 | 120.00 | 116.07 | 119.03 | 2.22 | 1.9% |
| Cholesterol | 11.20 | 11.62 | 11.48 | 11.44 | 11.05 | 11.36 | 0.23 | 2.0% |
| DOPC degradants by | | | | | | | | |
| RP-HPLC (mg/mL) | | | | | | | | |
| LPC | 1.46 | 1.48 | 1.56 | 1.51 | 1.48 | 1.50 | n/a | n/a |
| Oleic Acid | 0.60 | 0.57 | 0.59 | 0.62 | 0.50 | 0.58 | | |
| Cholesterol degradants | | | | | | | | |
| by RP-HPLC (mg/mL) | | | | | | | | |
| 7β-Hydroxycholesterol | n.d. | n.d. | n.d. | n.d. | n.d. | n/a | n/a | n/a |
| 7-Ketocholesterol | n.d. | n.d. | n.d. | n.d. | n.d. | | | |
| Lipid Impurities | | | | | | | | |
| (% Area) | | | | | | | | |
| POPC | 2.79% | 2.62% | 2.63% | 2.61% | 2.59% | 2.65% | n/a | n/a |
| Cholesterol impurity | int | int | int | int | int | | | |

n.d. = not detected
int = interference from oil components

The data demonstrates that the methods disclosed herein are reproducible in generating pharmaceutical-grade compositions with consistent concentrations of therapeutic agent, adjuvant and T-helper epitope.

### REFERENCES

1) Akbarzadeh, A.; Rezaei-Sadabady, R.; Davaran, S.; Joo, S. W.; Zarghami, N.; Hanifehpour, Y.; Samiei, M.; Kouhi, M.; and Nejati-Koshki, K. (2013) Liposome: classification, preparation, and applications, Nanoscale Res Lett. 8(1), 102.
2) Banga, A. K. Therapeutic Peptides and Proteins, Formulation, Processing and Delivery Systems (Lancaster, Pa.: Technomic Publishing Co., 1995).
3) Bobst, A. M.; Langemeier, P. W.; Torrence, P. F.; De Clercq, E. (1981) Interferon Induction by Poly(inosinic acid).Poly(cytidylic acid) Segmented by Spin-Labels, Biochemistry 20(16), 4798-4803.
4) Caruthers, M. H.; Beaucage, S. L.; Efcavitch, J. W.; Fisher, E. F.; Matteucci, M. D.; Stabinsky, Y. (1980) New chemical methods for synthesizing polynucleotides, Nucleic Acids Res. Symp. Ser. (7), 215-223.
5) Castle, J. C.: Kreiter, S.; Diekmann, J.; Lower, M.; van de Roemer, N.; de Graaf, J.; Selmi, A.; Diken, M.; Boegel, S.; Paret, C.; Koslowski, M.; Kuhn, A. N.; Britten, C. M.; Huber, C.; Turesi, O.; and Sahin, U. (2012) Exploiting the Mutanome for Tumor Vaccination. Cancer Res, 72(5): 1081-1091.
6) Celis, E.; Ou, D.; Otvos, L. Jr. (1988) Recognition of hepatitis B surface antigen by human T lymphocytes. Proliferative and cytotoxic responses to a major antigenic determinant defined by synthetic peptides, J. Immunol. 140, 1808-1815.
7) Chong, P.; Zobrist, G.; Sia, C.; Loosmore, S.; Klein, M. (1992) Identification of T- and B-cell epitopes of the S2 and S3 subunits of pertussis toxin by use of synthetic peptides, Infect Immun. 60, 4640-4647.
8) Coligan et al., ed. Current Protocols in Immunology, Wiley Interscience, 2007
9) Collins, P. L.; Olmsted, R. A.; Johnson, P. R. (1990) The small hydrophobic protein of human respiratory syncytial virus: comparison between antigenic subgroups A and B, J Gen Virol. 71(7), 1571-6.
10) Demotz, S.; Lanzavecchia, A.; Eisel, U.; Niemann, H.; Widmann, C.; Corradin, G. P. (1989) Delineation of several DR-restricted epitopes in tetanus toxin, J. Immunol. 142, 394-402.
11) Diethelm-Okita, B. M.; Okita, D. K.; Banaszak, L.; Conti-Fine, B. M. (2000) Universal epitopes for human CD4+ cells on tetanus and diphtheria toxins, J. Infect. Dis. 181, 1001-1009.
12) Flynn, J. L. (1994) Recombinant BCG as an antigen delivery system, Cell. Mol. Biol. 40 (suppl. 1), 31-36.
13) Frezard, F. (1999) Liposomes: from biophysics to the design of peptide vaccines, Braz. J. Med. Bio. Res. 32, 181-189.
14) Greene, J. J.; Alderfer, J. L.; Tazawa, I.; Tazawa, S.; Ts'o, P. O.; O'Malley, J. A.; Carter, W. A. (1978) Interferon induction and its dependence on the primary and secondary structure of poly(inosinic acid).poly(cytidylic acid), Biochemistry 17(20), 4214-4220.
15) Gregoriadis G. (1990) Immunological adjuvants: a role for liposomes, Immunol. Today 11, 89-97.
16) Hacohen, N.; Wu, C. J.; Fritsch, E. F.; Combination therapy with Neoantigen Vaccine, US20160339090A1.
17) Hagstrom, N. J.; Couto, L. B.; Scallan, C.; Burton, M.; McCleland, M. L.; Fields, P. A.; Arruda, V. R.; Herzog, R. W. and High, K. A. (2000) Blood, 95, 2536-2542.
18) Hendrix, C. W.; Margolick, J. B.; Petty, B. G.; Markham, R. B.; Nerhood, L.; Farzadegan, H.; Ts'o, P. O.; Lietman, P. S. (1993) Biologic effects after a single dose of poly(I):poly(C12U) in healthy volunteers, Antimicrob. Agents Chemother. 37(3), 429-435.
19) High, N.; Mounier, J.; Prévost, M. C.; Sansonetti, P. J. (1992) IpaB of Shigella flexneri causes entry into epithelial cells and escape from the phagocytic vacuole, EMBO J 11, 1991-1999.
20) Horn, T.; Vasser, M. P.; Struble, M. E.; Crea, R. (1980) Synthesis of oligonucleotides on cellulose. Part II: Design and synthetic strategy to the synthesis of 22 oligodeoxynucleotides coding for gastric inhibitory polypeptide (GIP), Nucleic Acids Symp. Ser. (7), 225-232.
21) Kawaoka, Y.; Yamnikova, S.; Chambers, T. M.; Lvov, D. K.; Webster, R. G. (1990) Molecular characterization of a new hemagglutinin, subtype H14, of influenza A virus, Virology 179(2), 759-767.
22) Kieny, M. P.; Lathe, R.; Drillien, R.; Spehner, D.; Skory, S.; Schmitt, D.; Wiktor, T.; Koprowski, H. and Lecocq, J. P. (1984) Expression of rabies virus glycoprotein from a recombinant vaccinia virus, Nature 312, 163-166.
23) Levey, D. L.; Castle, J. C.; Stein, R. B. and Buell, J. S. Vaccines for treatment and prevention of cancer, US20160331821A1.
24) Li, X.; Eastman, E. M.; Schwartz, R. J.; and Draghia-Akli, R. (1999) Synthetic muscle promoters: activities exceeding naturally occurring regulatory sequences, Nat. Biotechnol 17, 241-245.
25) Li, Z. L. and Paulin, D. (1991) High level desmin expression depends on a muscle-specific enhancer, J. Biol. Chem. 266, 6562-6570.
26) Li, Z. L. and Paulin, D. (1993) Different factors interact with myoblast-specific and myotube-specific enhancer regions of the human desmin gene, J. Biol. Chem. 268, 10403-10415.
27) Medaglini, D.; Pozzi, G.; King, T. P. and Fischetti, V. A. (1995) Mucosal and systemic immune responses to a recombinant protein expressed on the surface of the oral commensal bacterium Streptococcus gordonii after oral colonization, Proc. Natl. Acad. Sci. USA 92, 6868-6872.
28) Mekalanos, J. J.; Swartz, D. J.; Pearson, G. D. N.; Harford, N.; Groyne, F. and de wilde, M. (1983) Cholera toxin genes: nucleotide sequence, deletion analysis and vaccine development, Nature 306, 551-557.
29) Merrifield, B. (1997) Concept and early development of solid-phase peptide synthesis, Methods Enzymol. 289, 3-13.
30) Moyle, P. M.; Toth. I. (2008) Self-adjuvanting lipopeptide vaccines, Curr. Med. Chem. 15(5), 506-516.
31) Mullard A. (2016) The cancer vaccine resurgence, Nat Rev Drug Disc 15, 663-665.
32) Nakayama, K.; Kelly, S. M. and Curtis, R. III (1988) Construction of an Asd+expression cloning vector: stable maintenance and high level expression of cloned genes in a Salmonella strain, Bio/Technology 6, 693-697.
33) Roberge, J. Y.; Beebe, X.; Danishefsky, S. J. (1995) A strategy for a convergent synthesis of N-linked glycopeptides on a solid support, Science 269(5221), 202-204.
34) Remington's Pharmaceutical Sciences, Mack Publishing Company, Easton, Pa., USA 1985.
35) Sizemore, D. R.; Branstrom, A. A.; and Sadoff, J. C. (1995) Attenuated Shigella as a DNA Delivery Vehicle for DNA-Mediated Immunization, Science 270, 299-303.
36) Slingluff, C. L. Jr.; Yamshchikov, G.; Neese, P.; Galavotti, H.; Eastham, S.; Engelhard, V. H.; Kittlesen, D.; Deacon, D.; Hibbitts, S.; Grosh, W. W.; Petroni, G.; Cohen, R.; Wiernasz, C.; Patterson, J. W.; Conway, B. P.; Ross, W. G. (2001) Phase I trial of a melanoma vaccine with gp100(280-288) peptide and tetanus helper peptide in adjuvant: Immunologic and clinical outcomes, Clin Cancer Res. 7, 3012-3024.
37) Skwarczynski, M. and Toth, I. (2016) Peptide-based synthetic vaccines, Chem. Sci. 7, 842-854.
38) So, N. S. Y.; Ostrowski, M. A.; Gray-Owen, S. D. (2012) Vigorous Response of Human Innate Functioning IgM Memory B Cells upon Infection by Neisseria gonorrhoeae, J. Immunol. 188(8), 4008-4022.
39) Srivastava, P. (2015) Neoepitopes of Cancers: Looking Back, Looking Ahead, Cancer Immunol. Res. 3, 969-977.
40) Tartaglia, J.; Perkus, M. E.; Taylor, J.; Norton, E. K.; Audonnet, J. C.; Cox, W. I.; Davis, S. W.; van der Hoeven, J.; Meignier, B.; Riviere, M.; et al. (1992) NYVAC: a highly attenuated strain of vaccinia virus, Virology 188, 217-232.
41) Taylor, J.; Meignier, B.; Tartaglia, J.; Languet, B.; VanderHoeven, J.; Franchini, G.; Trimarchi, C.; and Paoletti E. (1995) Biological and immunogenic properties of a canarypox-rabies recombinant, ALVAC-RG (vCP65) in non-avian species, Vaccine 13, 539-549.
42) The United States Pharmacopoeia: The National Formulary (USP 24 NF19) published in 1999.
43) Webster, R. G.; Laver, W. G.; Air, G. M. Antigenic variation among type A influenza viruses, p. 127-168. In: Palese, P. & Kingsbury, D. W., eds. Genetics of influenza viruses. (New York: Springer-Verlag, 1983).
44) Zhenlin, L.; Lilienbaum, A.; Butler-Browne, G.; Paulin, D. (1989) Human desmin-coding gene: complete nucleotide sequence, characterization and regulation of expression during myogenesis and development, Gene 78, 243-254.

1. A method for preparing a dried preparation comprising lipids and therapeutic agents, said method comprising the steps of:
(a) providing a lipid vesicle particle preparation comprising lipid vesicle particles and at least one solubilized first therapeutic agent;
(b) sizing the lipid vesicle particle preparation to form a sized lipid vesicle particle preparation comprising sized lipid vesicle particles and said at least one solubilized first therapeutic agent, said sized lipid vesicle particles having a mean particle size of ≤120 nm and a polydispersity index (PDI) of ≤0.1;
(c) mixing the sized lipid vesicle particle preparation with at least one second therapeutic agent to form a mixture, wherein said at least one second therapeutic agent is solubilized in the mixture and is different from said at least one solubilized first therapeutic agent; and
(d) drying the mixture formed in step (c) to form a dried preparation comprising lipids and therapeutic agents.

2. The method of claim 1, wherein prior to step (b) the lipid vesicle particles are not sized.

3. The method of claim 1 or 2, wherein, in step (a), the lipid vesicle particles and the at least one solubilized first therapeutic agent are in sodium acetate or sodium phosphate.

4. The method of any one of claims 1 to 3, wherein, in step (a), the lipid vesicle particles and the at least one solubilized first therapeutic agent are in 25-250 mM sodium acetate having a pH in the range of 6.0-10.5 or 25-250 mM sodium phosphate having a pH in the range of 6.0-8.0.

5. The method of any one of claims 1 to 4, wherein, in step (a), the lipid vesicle particles and the at least one solubilized first therapeutic agent are in 50 mM sodium acetate having a pH of 6.0±1.0, 100 mM sodium acetate having a pH of 9.5±1.0, 50 mM sodium phosphate having a pH of 7.0±1.0 or 100 mM sodium phosphate having a pH of 6.0±1.0.

6. The method of any one of claims 1 to 5, wherein, in step (a), the lipid vesicle particles and the at least one solubilized first therapeutic agent are in 100 mM sodium acetate having a pH of 9.5±0.5.

7. The method of any one of claims 1 to 6, wherein, in step (a), the lipid vesicle particle preparation further comprises a solubilized adjuvant.

8. The method of any one of claims 1 to 6, wherein step (a) comprises:
(a1) providing a therapeutic agent stock comprising the at least one solubilized first therapeutic agent, and optionally further comprising a solubilized adjuvant; and
(a2) mixing the therapeutic agent stock with a lipid mixture to form the lipid vesicle preparation.

9. The method of claim 7 or 8, wherein the solubilized adjuvant is encapsulated in the lipid vesicle particles.

10. The method of any one of claims 7 to 9, wherein the adjuvant is a polyI:C polynucleotide adjuvant.

11. The method of any one of claims 1 to 10, wherein, in step (a), the at least one solubilized first therapeutic agent is encapsulated in the lipid vesicle particles.

12. The method of any one of claims 1 to 11, wherein each of the first and second therapeutic agents is independently selected from the group consisting of a peptide antigen, a DNA or RNA polynucleotide that encodes a polypeptide, a hormone, a cytokine, an allergen, a catalytic DNA (deoxyribozyme), a catalytic RNA (ribozyme), an antisense RNA, an interfering RNA, an antagomir, a small molecule drug, a biologic drug, an antibody, or a fragment or derivative of any one thereof; or a mixture thereof.

13. The method of any one of claims 1 to 12, wherein each of the first and second therapeutic agents is a peptide antigen.

14. The method of any one of claims 1 to 13, wherein, in step (a), one, two, three, four or five different solubilized first therapeutic agents are in the lipid vesicle particle preparation.

15. The method of any one of claims 1 to 14, wherein, in step (a), four different solubilized first therapeutic agents are in the lipid vesicle particle preparation.

16. The method of claim 15, wherein the four different solubilized first therapeutic agents are peptide antigens, wherein the first peptide antigen comprises the amino acid sequence FTELTLGEF (SEQ ID NO: 1); the second peptide antigen comprises the amino acid sequence LMLGEFLKL (SEQ ID NO: 2); the third peptide antigen comprises the amino acid sequence STFKNWPFL (SEQ ID NO: 3); and the fourth peptide antigen comprises the amino acid sequence LPPAWQPFL (SEQ ID NO: 4).

17. The method of any one of claims 1 to 16, wherein, in step (c), the sized lipid vesicle particle preparation is mixed with one, two, three, four or five different second therapeutic agents.

18. The method of any one of claims 1 to 17, wherein, in step (c), the sized lipid vesicle particle preparation is mixed with one second therapeutic agent.

19. The method of claim 18, wherein the one second therapeutic agent is a peptide antigen comprising the amino acid sequence RISTFKNWPK (SEQ ID NO: 6).

20. The method of any one of claims 1 to 19, wherein, in step (b), the lipid vesicle particle preparation of step (a) is sized by high pressure homogenization, sonication or membrane extrusion.

21. The method of claim 20, wherein, in step (b), the lipid vesicle particle preparation of (step (a) is sized by extrusion through a 0.2 μm polycarbonate membrane followed by extrusion through a 0.1 μm polycarbonate membrane.

22. The method of claim 21, wherein the lipid vesicle particle preparation of step (a) is sized by extrusion through the 0.2 μm polycarbonate membrane 20 to 40 times and extrusion through the 0.1 μm polycarbonate membrane 10 to 20 times.

23. The method of any one of claims 20 to 22, wherein the membrane extrusion is performed at 1000 to 5000 psi back pressure.

24. The method of any one of claims 20 to 23, wherein the at least one solubilized first therapeutic agent is soluble at alkaline pH during high pressure membrane extrusion at about 5000 psi.

25. The method of any one of claims 1 to 24, wherein the at least one second therapeutic agent is solubilized in mild acetic acid prior to mixing with the sized lipid vesicle particle preparation in step (c).

26. The method of any one of claims 1 to 25, wherein step (c) further comprises mixing, in any order, at least one T-helper epitope with the sized lipid vesicle particle preparation and the at least one second therapeutic agent, wherein the at least one T-helper epitope is solubilized in the mixture.

27. The method of claim 26, wherein the T-helper epitope comprises the amino acid sequence AQYIKANSKFIGITEL (SEQ ID NO: 5).

28. The method of claim 26 or 27, wherein step (c) comprises:
(c1) providing a one or more therapeutic agent stocks comprising a solubilized second therapeutic agent, and a stock comprising the T-helper epitope; and
(c2) mixing the stocks with the sized lipid vesicle particles to form the mixture.

29. The method of claim 28, wherein the one or more therapeutic agent stocks are prepared in mild acetic acid.

30. The method of any one of claims 1 to 29, wherein the mean particle size of the sized lipid vesicle particles is between about 80 nm and about 120 nm.

31. The method of any one of claims 1 to 30, wherein the mean particle size of the sized lipid vesicle particles is about 80 nm, about 81 nm, about 82 nm, about 83 nm, about 84 nm, about 85 nm, about 86 nm, about 87 nm, about 88 nm, about 89 nm, about 90 nm, about 91 nm, about 92 nm, about 93 nm, about 94 nm, about 95 nm, about 96 nm, about 97 nm, about 98 nm, about 99 nm, about 100 nm, about 101 nm, about 102 nm, about 103 nm, about 104 nm, about 105 nm, about 106 nm, about 107 nm, about 108 nm, about 109 nm, about 110 nm, about 111 nm, about 112 nm, about 113 nm, about 114 nm or about 115 nm.

32. The method of any one of claims 1 to 31, wherein the mean particle size of the sized lipid vesicle particles is ≤100 nm.

33. The method of any one of claims 1 to 32, wherein the lipid vesicle particles comprise a synthetic lipid.

34. The method of claim 33, wherein the lipid vesicle particles comprise synthetic dioleoyl phosphatidylcholine (DOPC) or synthetic DOPC and cholesterol.

35. The method of claim 34, wherein the lipid vesicle particles comprise synthetic DOPC and cholesterol at a DOPC:cholesterol ratio of 10:1 (w/w).

36. The method of any one of claims 1 to 35, wherein the lipid vesicle particles are liposomes.

37. The method of claim 36, wherein the liposomes are unilamellar, multilamellar, or a mixture thereof.

38. The method of any one of claims 1 to 37 further comprising a step of sterile filtration of the mixture formed in step (c) prior to drying.

39. The method of any one of claims 1 to 38 further comprising, between steps (c) and (d), a step of confirming that the sized lipid vesicle particles still have a mean particle size of ≤120 nm and a polydispersity index (PDI) of ≤0.1.

40. The method of any one of claims 1 to 39, wherein the drying is performed by lyophilization, spray freeze-drying, or spray drying.

41. The method of claim 40, wherein the drying is performed by lyophilization.

42. A method for preparing a pharmaceutical composition comprising solubilizing the dried preparation obtained by the method of any one of claims 1 to 41 in a hydrophobic carrier.

43. The method of claim 42, wherein the hydrophobic carrier is mineral oil or a mannide oleate in mineral oil solution.

44. The method of claim 42 or 43, wherein the hydrophobic carrier is Montanide® ISA 51.

45. A pharmaceutical composition prepared by the method of any one of claims 42 to 44.

46. The pharmaceutical composition of claim 45, wherein the lipids are in the form of one or more lipid-based structures having a single layer lipid assembly in the hydrophobic carrier.

47. The pharmaceutical composition of claim 46, wherein, in the hydrophobic carrier, the lipids are in the form of reverse micelles and/or aggregates of lipids with the hydrophobic part of the lipids oriented outwards toward the hydrophobic carrier and the hydrophilic part of the lipids aggregating as a core.

48. The pharmaceutical composition of claim 46 or 47, wherein the size of the lipid-based structures is between about 5 nm to about 10 nm in diameter.

49. A stable, water-free pharmaceutical composition comprising one or more lipid-based structures having a single layer lipid assembly, at least two different therapeutic agents, and a hydrophobic carrier.

50. The pharmaceutical composition of claim 49, wherein the therapeutic agents are independently selected from the group consisting of a peptide antigen, a DNA or RNA polynucleotide that encodes a polypeptide, a hormone, a cytokine, an allergen, a catalytic DNA (deoxyribozyme), a catalytic RNA (ribozyme), an antisense RNA, an interfering RNA, an antagomir, a small molecule drug, a biologic drug, an antibody, or a fragment or derivative of any one thereof; or a mixture thereof.

51. The pharmaceutical composition of claim 49 or 50, wherein the therapeutic agents are peptide antigens.

52. The pharmaceutical composition of claim 51, which comprises two, three, four, five or more different peptide antigens.

53. The pharmaceutical composition of claim 52, which comprises five different peptide antigens.

54. The pharmaceutical composition of claim 53, wherein the first peptide antigen comprises the amino acid sequence FTELTLGEF (SEQ ID NO: 1); the second peptide antigen comprises the amino acid sequence LMLGEFLKL (SEQ ID NO: 2); the third peptide antigen comprises the amino acid sequence STFKNWPFL (SEQ ID NO: 3); the fourth peptide antigen comprises the amino acid sequence LPPAWQPFL (SEQ ID NO: 4); and the fifth peptide antigen comprising the amino acid sequence RISTFKNWPK (SEQ ID NO: 6).

55. The pharmaceutical composition of any one of claims 51 to 54, wherein each of the peptide antigens is, independently, at a concentration of between about 0.1 μg/μl and about 5.0 μg/μl.

56. The pharmaceutical composition of any one of claims 51 to 55, which comprises five different peptide antigens, each at a concentration of at least about 1.0 μg/μl.

57. The pharmaceutical composition of any one of claims 49 to 56, further comprising one or both of a T-helper epitope and an adjuvant.

58. The pharmaceutical composition of claim 57, wherein the T-helper epitope comprises the amino acid sequence AQYIKANSKFIGITEL (SEQ ID NO: 5) and the adjuvant is a polyI:C polynucleotide adjuvant.

59. The pharmaceutical composition of any one of claims 49 to 58, wherein the hydrophobic carrier is mineral oil or a mannide oleate in mineral oil solution.

60. The pharmaceutical composition of any one of claims 49 to 59, wherein the hydrophobic carrier is Montanide® ISA 51.

61. The pharmaceutical composition of any one of claims 49 to 60, wherein the one or more lipid-based structures having a single layer lipid assembly comprise aggregates of lipids with the hydrophobic part of the lipids oriented outwards toward the hydrophobic carrier and the hydrophilic part of the lipids aggregating as a core.

62. The pharmaceutical composition of any one of claims 49 to 61, wherein the one or more lipid-based structures having a single layer lipid assembly comprise reverse micelles.

63. The pharmaceutical composition of any one of claims 49 to 62, wherein the size of the lipid-based structures is between about 5 nm to about 10 nm in diameter.

64. The pharmaceutical composition of any one of claims 49 to 63, wherein one or more of the therapeutic agents are inside the lipid-based structures.

65. The pharmaceutical composition of any one of claims 49 to 64, wherein one or more of the therapeutic agents are outside the lipid-based structures.

66. The pharmaceutical composition of any one of claims 49 to 65, which is a clear solution.

67. The pharmaceutical composition of any one of claims 49 to 66, which has no visible precipitate.

68. A method of inducing an antibody and/or CTL immune response in a subject comprising administering to the subject the pharmaceutical composition of any one of claims 45 to 67.

69. The method of claim 68, which is for treating cancer or an infectious disease.

70. Use of the pharmaceutical composition of any one of claims 45 to 67 for inducing an antibody and/or CTL immune response in a subject.

71. The use of claim 70, which is for the treatment of cancer or an infectious disease.

72. A kit for preparing a pharmaceutical composition for inducing an antibody and/or CTL immune response, the kit comprising:
a container comprising a dried preparation prepared by the method of any one of claims 1 to 41; and
a container comprising a hydrophobic carrier.

73. The kit of claim 72, wherein the dried preparation comprises five or more different peptide antigens.

74. The kit of claim 72 or 73, wherein the hydrophobic carrier is mineral oil or a mannide oleate in mineral oil solution.


SEQUENCE LISTING (33 sequences)

SEQ ID NO: 1 - protein, 9 aa, Artificial Sequence
  Survivin HLA-A1 (modified)
FTELTLGEF

SEQ ID NO: 2 - protein, 9 aa, Artificial Sequence
  Survivin HLA-A2 (modified)
LMLGEFLKL

SEQ ID NO: 3 - protein, 9 aa, Artificial Sequence
  Survivin HLA-A24
STFKNWPFL

SEQ ID NO: 4 - protein, 9 aa, Artificial Sequence
  Survivin HLA-B7
LPPAWQPFL

SEQ ID NO: 5 - protein, 16 aa, Artificial Sequence
  A16Y T-helper epitope
AQYIKANSKFIGITEL

SEQ ID NO: 6 - protein, 10 aa, Artificial Sequence
  Survivin HLA-A3 (modified)
RISTFKNWPK

SEQ ID NO: 7 - protein, 23 aa, Artificial Sequence
  RSV SHeA
NKLCEYNVFHNKTFELPRARVNT

SEQ ID NO: 8 - protein, 24 aa, Artificial Sequence
  RSV SHeB
NKLSEHKTFCNKTLEQGQMYQINT

SEQ ID NO: 9 - protein, 9 aa, Artificial Sequence
  HPV 16E7
RAHYNIVTF

SEQ ID NO: 10 - protein, 9 aa, Artificial Sequence
  HPV Y9T
YMLNLGPET

SEQ ID NO: 11 - protein, 10 aa, Artificial Sequence
  HIV RGP10
RGPGRAFVTI

SEQ ID NO: 12 - protein, 9 aa, Artificial Sequence
  HIV AMQ9
AMQMLKETI

SEQ ID NO: 13 - protein, 64 aa, Artificial Sequence
  RSV Subtype A
MENTSITIEFSSKFWPYFTLIHMITTIISLLIIISIMIAILNKLCEYNVFHNKTFELPRARVNT

SEQ ID NO: 14 - protein, 65 aa, Artificial Sequence
  RSV Subtype B
MGNTSITIEFTSKFWPYFTLIHMILTLISLLIIITIMIAILNKLSEHKTFCNKTLEQGQMYQINT

SEQ ID NO: 15 - protein, 23 aa, Artificial Sequence
  RSV SHeA C45S
NKLSEYNVFHNKTFELPRARVNT

SEQ ID NO: 16 - protein, 40 aa, Artificial Sequence
  RSV bSHeA
NKLCDLNDHHTNSLDIRTRLRNDTQLITRAHEGSINQSSN

SEQ ID NO: 17 - protein, 40 aa, Artificial Sequence
  RSV bSHeA C45S
NKLSDLNDHHTNSLDIRTRLRNDTQLITRAHEGSINQSSN

SEQ ID NO: 18 - protein, 24 aa, Artificial Sequence
  RSV SHeB C51S
NKLSEHKTFSNKTLEQGQMYQINT

SEQ ID NO: 19 - protein, 24 aa, Artificial Sequence
  RSV SHeB C45S
NKLCEHKTFSNKTLEQGQMYQINT

SEQ ID NO: 20 - protein, 29 aa, Artificial Sequence
  RSV L-SHeB C51S
CGGGSNKLSEHKTFSNKTLEQGQMYQINT

SEQ ID NO: 21 - DNA, 429 bp, Homo sapiens
atgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggattga

SEQ ID NO: 22 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 23 - protein, 9 aa, Artificial Sequence
  Survivin HLA-A1
FEELTLGEF

SEQ ID NO: 24 - protein, 9 aa, Artificial Sequence
  Survivin HLA-A2
LTLGEFLKL

SEQ ID NO: 25 - protein, 10 aa, Artificial Sequence
  Survivin HLA-A3
RISTFKNWPF

SEQ ID NO: 26 - protein, 27 aa, Artificial Sequence
  Mut25
STANYNTSHLNNDVWQIFENPVDWKEK

SEQ ID NO: 27 - protein, 27 aa, Artificial Sequence
  Mut30
PSKPSFQEFVDWENVSPELNSTDQPFL

SEQ ID NO: 28 - protein, 27 aa, Artificial Sequence
  Mut44
EFKHIKAFDRTFANNPGPMVVFATPGM

SEQ ID NO: 29 - protein, 13 aa, Artificial Sequence
  PADRE T-helper epitope
  misc_feature (3)..(3): Xaa may be cyclohexylalanyl
AKXVAAWTLKAAA

SEQ ID NO: 30 - protein, 21 aa, Artificial Sequence
  F21E T-helper epitope
FNNFTVSFWLRVPKVSASHLE

SEQ ID NO: 31 - DNA, 20 bp, Artificial Sequence
  CpG oligonucleotide
tccatgacgttcctgacgtt

SEQ ID NO: 32 - DNA, 26 bp, Artificial Sequence
  PolyIC oligonucleotide (dIdC)
  modified_base (1)..(1): Inosine
  misc_feature (1)..(1): n is a, c, g, or t
  modified_base (3)..(3): Inosine
  misc_feature (3)..(3): n is a, c, g, or t
  modified_base (5)..(5): Inosine
  misc_feature (5)..(5): n is a, c, g, or t
  modified_base (7)..(7): Inosine
  misc_feature (7)..(7): n is a, c, g, or t
  modified_base (9)..(9): Inosine
  misc_feature (9)..(9): n is a, c, g, or t
  modified_base (11)..(11): Inosine
  misc_feature (11)..(11): n is a, c, g, or t
  modified_base (13)..(13): Inosine
  misc_feature (13)..(13): n is a, c, g, or t
  modified_base (15)..(15): Inosine
  misc_feature (15)..(15): n is a, c, g, or t
  modified_base (17)..(17): Inosine
  misc_feature (17)..(17): n is a, c, g, or t
  modified_base (19)..(19): Inosine
  misc_feature (19)..(19): n is a, c, g, or t
  modified_base (21)..(21): Inosine
  misc_feature (21)..(21): n is a, c, g, or t
  modified_base (23)..(23): Inosine
  misc_feature (23)..(23): n is a, c, g, or t
  misc_feature (25)..(25): n is a, c, g, or t
ncncncncncncncncncncncncnc

SEQ ID NO: 33 - protein, 6 aa, Artificial Sequence
  Palmitic Acid Adjuvant
  misc_feature (1)..(1): Linked to PAM2 or PAM3
CSKKKK